(19) United States
(12) Patent Application Publication
(10) Pub. No.: US 2026/0294778 A1
(43) Pub. Date: Oct. 1, 2026

(54) AMPHIPHILIC GLUCAN ESTER DERIVATIVES

(71) Applicant: Nutrition & Biosciences USA 4, Inc., Wilmington, DE (US)

(72) Inventor: Zhengzheng Huang, Hockessin, DE (US)

(21) Appl. No.: 18/881,346

(22) PCT Filed: Jul. 11, 2023
(86) PCT No.: PCT/US2023/069938

§ 371 Date: Jan. 6, 2025

Related U.S. Application Data
(60) Provisional application No. 63/359,965, filed on Jul. 11, 2022.

(51) Int. Cl. A61K 8/73; A61Q 5/06; C08B 37/00

(52) U.S. Cl. CPC A61K 8/73; A61Q 5/06; C08B 37/0009

(57) ABSTRACT

Compositions are disclosed herein comprising an ester derivative of a glucan, wherein the glucan has a degree of substitution (DoS) up to about 3.0 with at least two organic groups that are individually ester-linked to the glucan, wherein (i) at least one of the organic groups is a cationic organic group, and (ii) at least one of the organic groups is a hydrophobic organic group. Methods are further disclosed for preparing these compositions, as well as various applications of using them.

This application claims the benefit of U.S. Provisional Appl. No. 63/359,965 (filed Jul. 11, 2022), which is incorporated herein by reference in its entirety.

### FIELD

The present disclosure is in the field of polysaccharide derivatives. For example, the disclosure pertains to amphiphilic glucan ester derivatives, and use thereof in various applications.

### BACKGROUND

Driven by a desire to find new structural polysaccharides using enzymatic syntheses or genetic engineering of microorganisms, researchers have discovered oligosaccharides and polysaccharides that are biodegradable and that can be made economically from renewably-sourced feedstocks. Further work has shown that such polysaccharides can be chemically modified (derivatized) to have additional utilities in areas such as personal care, household care, industrial care, pharmaceuticals and food. For example, ethers and esters of alpha-glucan comprising alpha-1,3 glycosidic linkages have been disclosed to have various applications (e.g., U.S. Patent Appl. Publ. Nos. 2016/0304629, 2016/0311935, 2017/0204232, 2014/0187767, 2020/0308371). Various derivatives of alpha-glucan comprising alpha-1,6 glycosidic linkages, and applications for use thereof, have also been disclosed (e.g., U.S. Patent Appl. Publ. Nos. 2018/0312781, 2018/0237816, 2018/0282385).

Despite these advances, some glucan derivatives with a desired utility have poor biodegradability profiles by virtue of having an elevated level of derivatization. While some glucans with low levels of derivatization exhibit better biodegradability, such products often fail to deliver optimal, or even adequate, activity. Thus, there remains a need for product ingredients that are not only renewable, but also biodegradable and that provide performance that is equal to, or better than, the performance of products with synthetic components. Amphiphilic glucan ester derivatives (and other compounds) are disclosed herein to address this need.

### SUMMARY

In one embodiment, the present disclosure concerns a composition comprising an ester derivative of a glucan, wherein the glucan has a degree of substitution (DoS) up to about 3.0 with at least two organic groups that are individually ester-linked to the glucan, wherein (i) at least one of the organic groups is a cationic organic group, and (ii) at least one of the organic groups is a hydrophobic organic group.

In another embodiment, the present disclosure concerns a method of producing an ester derivative of a glucan. Such a method comprises: (a) contacting a glucan with at least two esterification agents, wherein at least one of the esterification agents comprises a cationic organic group, wherein at least one of the esterification agents comprises a hydrophobic organic group, wherein at least one cationic organic group and at least one hydrophobic organic group are esterified to the glucan, thereby producing an ester derivative of the glucan, wherein the ester derivative of the glucan has a degree of substitution (DoS) up to about 3.0 with the cationic organic group and the hydrophobic organic group, and (b) optionally, isolating the ester derivative of the glucan produced in step (a).

In another embodiment, the present disclosure concerns a method of styling hair. Such a method comprises at least steps (a) and (b), or steps (c) and (d), as follows: (a) contacting hair with a glucan ester derivative herein, thereby providing treated hair, and (b) putting the treated hair into a desired form; or (c) putting hair into a desired form, and (d) contacting the hair of step (c) with a glucan ester derivative herein, thereby providing treated hair; and (e) optionally, removing solvent, if present, that was used to deliver the glucan ester derivative to the hair in step (a) or (d).

### BRIEF DESCRIPTION OF THE DRAWINGS

FIG. 1: Shown is a biodegradability profile of betaine-modified alpha-1,3-glucan ester. RN3 and RN7 curves represent duplicate tests using the same glucan ester sample. Refer to Example 3.

### DETAILED DESCRIPTION

The disclosures of all cited patent and non-patent literature are incorporated herein by reference in their entirety.

Unless otherwise disclosed, the terms “a” and “an” as used herein are intended to encompass one or more (i.e., at least one) of a referenced feature.

Where present, all ranges are inclusive and combinable, except as otherwise noted. For example, when a range of “1 to 5” (i.e., 1-5) is recited, the recited range should be construed as including ranges “1 to 4”, “1 to 3”, “1-2”, “1-2 & 4-5”, “1-3 & 5”, and the like. The numerical values of the various ranges in the present disclosure, unless expressly indicated otherwise, are stated as approximations as though the minimum and maximum values within the stated ranges were both proceeded by the word “about”. In this manner, slight variations above and below the stated ranges can typically be used to achieve substantially the same results as values within the ranges. Also, the disclosure of these ranges is intended as a continuous range including each and every value between the minimum and maximum values.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

It is to be appreciated that certain features of the present disclosure, which are, for clarity, described above and below in the context of aspects/embodiments, may also be provided in combination in a single element. Conversely, various features of the disclosure that are, for brevity, described in the context of a single aspect/embodiment, can also be provided separately or in any sub-combination.

The term “polysaccharide” (or “glycan”) means a polymeric carbohydrate molecule composed of long chains of monosaccharide units bound together by glycosidic linkages and on hydrolysis gives the polysaccharide's constituent monosaccharides and/or oligosaccharides. A polysaccharide herein can be linear or branched, and/or can be a homopolysaccharide (comprised of only one type of constituent monosaccharide) or heteropolysaccharide (comprised of two or more different constituent monosaccharides). Examples of polysaccharides herein include glucan (polyglucose) and soy polysaccharide.

A “glucan” herein is a type of polysaccharide that is a polymer of glucose (polyglucose). A glucan can be comprised of, for example, about, or at least about, 90%, 91%, 92%, 93%, 94%, 95%, 96%, 97%, 98%, 99%, or 100% by weight glucose monomeric units. Examples of glucans herein are alpha-glucan and beta-glucan.

The terms “alpha-glucan”, “alpha-glucan polymer” and the like are used interchangeably herein. An alpha-glucan is a polymer comprising glucose monomeric units linked together by alpha-glycosidic linkages. In typical aspects, the glycosidic linkages of an alpha-glucan herein are about, or at least about, 80%, 81%, 82%, 83%, 84%, 85%, 86%, 87%, 88%, 89%, 90%, 91%, 92%, 93%, 94%, 95%, 96%, 97%, 98%, 99%, or 100% alpha-glycosidic linkages. Examples of alpha-glucan polymers herein include alpha-1,3-glucan, alpha-1,4-glucan, and alpha-1,6-glucan.

The terms “beta-glucan”, “beta-glucan polymer” and the like are used interchangeably herein. A beta-glucan is a polymer comprising glucose monomeric units linked together by beta-glycosidic linkages. In typical aspects, the glycosidic linkages of a beta-glucan herein are about, or at least about, 80%, 81%, 82%, 83%, 84%, 85%, 86%, 87%, 88%, 89%, 90%, 91%, 92%, 93%, 94%, 95%, 96%, 97%, 98%, 99%, or 100% beta-glycosidic linkages. Examples of beta-glucan polymers herein include beta-1,3-glucan, beta-1,4-glucan, and beta-1,6-glucan.

The term “saccharide” and other like terms herein refer to monosaccharides and/or disaccharides/oligosaccharides, unless otherwise noted. A “disaccharide” herein refers to a carbohydrate having two monosaccharides joined by a glycosidic linkage. An “oligosaccharide” herein can refer to a carbohydrate having 3 to 15 monosaccharides, for example, joined by glycosidic linkages. An oligosaccharide can also be referred to as an “oligomer”. Monosaccharides (e.g., glucose and/or fructose) comprised within disaccharides/oligosaccharides can be referred to as “monomeric units”, “monosaccharide units”, or other like terms.

The terms “alpha-1,3-glucan”, “poly alpha-1,3-glucan”, “alpha-1,3-glucan polymer” and the like are used interchangeably herein. Alpha-1,3-glucan is an alpha-glucan comprising glucose monomeric units linked together by glycosidic linkages, wherein at least about 50% of the glycosidic linkages are alpha-1,3. Alpha-1,3-glucan in some aspects comprises about, or at least about, 90%, 95%, or 100% alpha-1,3 glycosidic linkages. Most or all of the other linkages, if present, in alpha-1,3-glucan herein typically are alpha-1,6, though some linkages may also be alpha-1,2 and/or alpha-1,4. Alpha-1,3-glucan herein is typically water-insoluble.

The terms “alpha-1,6-glucan”, “poly alpha-1,6-glucan”, “alpha-1,6-glucan polymer”, “dextran”, and the like herein refer to a water-soluble alpha-glucan comprising glucose monomeric units linked together by glycosidic linkages, wherein at least about 50% of the glycosidic linkages are alpha-1,6. Alpha-1,6-glucan in some aspects comprises about, or at least about, 90%, 95%, or 100% alpha-1,6 glycosidic linkages. Other linkages that can be present in alpha-1,6-glucan include alpha-1,2, alpha-1,3, and/or alpha-1,4 linkages.

The terms “alpha-1,4-glucan”, “poly alpha-1,4-glucan”, “alpha-1,4-glucan polymer” and the like are used interchangeably herein. Alpha-1,4-glucan is an alpha-glucan comprising glucose monomeric units linked together by glycosidic linkages, wherein at least about 50% of the glycosidic linkages are alpha-1,4. Alpha-1,4-glucan in some aspects comprises about, or at least about, 90%, 95%, or 100% alpha-1,4 glycosidic linkages. Most or all of other linkages (if present) in alpha-1,4-glucan herein typically are alpha-1,6 (typically forming a branch), but can also be alpha-1,2 and/or alpha-1,3. Examples of alpha-1,4-glucan herein include amylose, amylopectin, and starch.

The terms “beta-1,4-glucan”, “poly beta-1,4-glucan”, “beta-1,4-glucan polymer”, “cellulose”, and the like are used interchangeably herein. Beta-1,4-glucan is a water-insoluble beta-glucan comprising glucose monomeric units linked together by glycosidic linkages, wherein about 100% of the glycosidic linkages are beta-1,4. Beta-1,4-glucan can be as disclosed, for example, in U.S. Pat. Appl. Publ. No. 2018/0334696.

The terms “beta-1,3-glucan”, “poly beta-1,3-glucan”, “beta-1,3-glucan polymer” and the like are used interchangeably herein. Beta-1,3-glucan is a beta-glucan comprising glucose monomeric units linked together by glycosidic linkages, wherein at least about 50% of the glycosidic linkages are beta-1,3. Beta-1,3-glucan in some aspects comprises about, or at least about, 90%, 95%, or 100% beta-1,3 glycosidic linkages. Most or all of other linkages (if present) in beta-1,3-glucan herein typically are beta-1,6 (typically forming a branch). Beta-1,3-glucan can be as disclosed, for example, in U.S. Pat. Appl. Publ. No. 2014/0287919 and Stone, B. A. (2009, Chemistry of Beta-Glucans, In Antony Bacic et al., Eds., Chemistry, Biochemistry, and Biology of 1-3 Beta Glucans and Related Polysaccharides, Academic Press, Burlington, MA), which are incorporated herein by reference.

The terms “soy polysaccharide” and “soy fiber” are used interchangeably herein, and refer to high molecular weight, water-insoluble polysaccharide material that can be obtained from soybeans. Typically, soy polysaccharide is obtained from cell wall structural components of soybeans. Soy polysaccharide herein can be as disclosed, for example, in U.S. Pat. Appl. Publ. No. 2018/0079832, which is incorporated herein by reference.

An “alpha-1,2 branch” (and like terms) as referred to herein typically comprises a glucose that is alpha-1,2-linked to a dextran backbone; thus, an alpha-1,2 branch herein can also be referred to as an alpha-1,2,6 linkage. An alpha-1,2 branch herein typically has one glucose group (can optionally be referred to as a pendant glucose).

An “alpha-1,3 branch” (and like terms) as referred to herein typically comprises a glucose that is alpha-1,3-linked to a dextran backbone; thus, an alpha-1,3 branch herein can also be referred to as an alpha-1,3,6 linkage. An alpha-1,3 branch herein typically has one glucose group (can optionally be referred to as a pendant glucose).

An “alpha-1,4 branch” (and like terms) as referred to herein typically comprises a glucose that is alpha-1,4-linked to a dextran backbone; thus, an alpha-1,4 branch herein can also be referred to as an alpha-1,4,6 linkage. An alpha-1,4 branch herein typically has one glucose group (can optionally be referred to as a pendant glucose).

The percent branching in a polysaccharide herein refers to that percentage of all the linkages in the polysaccharide that represent branch points. For example, the percent of alpha-1,3 branching in an alpha-glucan herein refers to that percentage of all the linkages in the glucan that represent alpha-1,3 branch points. Except as otherwise noted, linkage percentages disclosed herein are based on the total linkages of a polysaccharide, or the portion of a polysaccharide for which a disclosure specifically regards.

The terms “linkage”, “glycosidic linkage”, “glycosidic bond” and the like refer to the covalent bonds connecting the sugar monomers within a saccharide compound (oligosaccharides and/or polysaccharides). Examples of glycosidic linkages include 1,6-alpha-D-glycosidic linkages (herein also referred to as “alpha-1,6” linkages), 1,3-alpha-D-glycosidic linkages (herein also referred to as “alpha-1,3” linkages), 1,4-alpha-D-glycosidic linkages (herein also referred to as “alpha-1,4” linkages), and 1,2-alpha-D-glycosidic linkages (herein also referred to as “alpha-1,2” linkages).

The glycosidic linkage profile of a polysaccharide or derivative thereof can be determined using any method known in the art. For example, a linkage profile can be determined using methods using nuclear magnetic resonance (NMR) spectroscopy (e.g., ¹³C NMR and/or ¹H NMR). These and other methods that can be used are disclosed in, for example, Food Carbohydrates: Chemistry, Physical Properties, and Applications (S. W. Cui, Ed., Chapter 3, S. W. Cui, Structural Analysis of Polysaccharides, Taylor & Francis Group LLC, Boca Raton, FL, 2005), which is incorporated herein by reference.

The term “molar substitution” (M.S.) as used herein refers to the moles of an organic group per monomeric unit of a polysaccharide derivative herein. It is noted that the molar substitution value for a polysaccharide derivative, for example, may have a very high upper limit, for example in the hundreds or even thousands.

The “molecular weight” of a polysaccharide or polysaccharide derivative herein can be represented as weight-average molecular weight (Mw) or number-average molecular weight (Mn), the units of which are in Daltons (Da) or grams/mole. Alternatively, molecular weight can be represented as DPw (weight average degree of polymerization) or DPn (number average degree of polymerization). The molecular weight of smaller polysaccharide polymers such as oligosaccharides can optionally be provided as “DP” (degree of polymerization), which simply refers to the number of monomers comprised within the polysaccharide; “DP” can also characterize the molecular weight of a polymer on an individual molecule basis. Various means are known in the art for calculating these various molecular weight measurements such as with high-pressure liquid chromatography (HPLC), size exclusion chromatography (SEC), or gel permeation chromatography (GPC).

As used herein, Mw can be calculated as Mw=ΣNiMi²/ΣNiMi; where Mi is the molecular weight of an individual chain i and Ni is the number of chains of that molecular weight. Besides SEC, the Mw of a polymer can be determined by other techniques such as static light scattering, mass spectrometry, MALDI-TOF (matrix-assisted laser desorption/ionization time-of-flight), small angle X-ray or neutron scattering, or ultracentrifugation. As used herein, Mn can be calculated as Mn=ΣNiMi/ΣNi where Mi is the molecular weight of a chain i and Ni is the number of chains of that molecular weight. Besides SEC, the Mn of a polymer can be determined by various colligative property methods such as vapor pressure osmometry, end-group determination by spectroscopic methods such as proton NMR, proton FTIR, or UV-Vis. As used herein, DPw and DPn can be calculated from Mw and Mn, respectively, by dividing them by molar mass of the one monomer unit M₁. In the case of unsubstituted glucan polymer, M₁=162. In the case of a substituted (derivatized) glucan polymer, M₁=162+M_{f}×DoS, where M_{f} is molar mass of the substituting group, and DoS is degree of substitution (average number of substituted groups per one glucose unit of the glucan polymer).

A “polysaccharide derivative” (and like terms) herein (e.g., a glucan derivative such as an alpha- or beta-glucan derivative) typically refers to a polysaccharide that has been substituted with at least one type of organic group (e.g., an acyl group herein). The degree of substitution (DoS) of a polysaccharide derivative herein can be up to about 3.0 (e.g., about 0.001 to about 3.0). An organic group herein that is an acyl group is linked to a polysaccharide derivative via ester linkage. A precursor of a polysaccharide derivative herein typically refers to the non-derivatized polysaccharide used to make the derivative (can also be referred to as the polysaccharide portion of the derivative). An organic group herein that is an acyl group can be positively charged (cationic) or hydrophobic; generally, a cationic charge can be as it exists when the organic group is in an aqueous composition herein, further taking into account the pH of the aqueous composition (in some aspects, the pH can be 4-10 or 5-9, or any pH as disclosed herein).

The term “degree of substitution” (DoS, or DS) as used herein refers to the average number of hydroxyl groups that are substituted with one or more types of organic group in each monomeric unit of a polysaccharide derivative. The DoS of a polysaccharide derivative herein can be stated with reference to the DoS of a specific substituent, or the overall DoS, which is the sum of the DoS values of different substituent types (e.g., if a mixed ester). Unless otherwise disclosed, when DoS is not stated with reference to a specific substituent type(s), the overall DoS is meant.

The terms “glucan ester derivative”, “glucan ester compound”, “glucan ester” and the like are used interchangeably herein. A glucan ester derivative herein typically is glucan that has been esterified with one or more cationic (positively charged) organic groups (i.e., cationic acyl groups) and one or more hydrophobic organic groups (i.e., hydrophobic acyl groups) such that the derivative has a DoS with all these organic groups of up to about 3.0. Such a glucan ester can optionally be characterized herein as an amphiphilic glucan ester by virtue of having one or more different hydrophilic organic groups (in particular, one or more cationic organic groups) and one or more different hydrophobic organic groups. A glucan ester derivative is termed an “ester” herein by virtue of comprising the substructure —C_{G}—O—CO—C—, where “—C_{G}—” represents a carbon atom of a monomeric unit (e.g., glucose) of the glucan ester derivative (where such carbon atom was bonded to a hydroxyl group [—OH] in the polysaccharide precursor of the ester), and where “—CO—C—” is comprised in the acyl group.

The term “hydrophobic” herein can characterize a substituent organic group (substituent acyl group) that is nonpolar and has little or no affinity to water, and tends to repel water.

The term “hydrophilic” herein can characterize a substituent organic group (substituent acyl group) that is polar and has affinity to interact with polar solvents, in particular with water, or with other polar groups. A hydrophilic group tends to attract water. A cationic organic group (cationic acyl group) herein is an example of a hydrophilic organic group.

The terms “esterification reaction”, “esterification reaction composition” and the like in some aspects refer to a reaction comprising at least a glucan as presently disclosed, two or more esterification agents and typically a solvent. A reaction is placed under suitable conditions (e.g., solvent, time, temperature) for esterification of hydroxyl groups of glucose monomeric units of glucan with at least one cationic organic group (cationic acyl group) and at least one hydrophobic organic group (hydrophobic acyl group) provided from the esterification agents, thereby yielding an amphiphilic glucan ester compound/derivative.

The terms “aqueous liquid”, “aqueous fluid”, “aqueous conditions”, “aqueous setting”, “aqueous system” and the like as used herein can refer to water or an aqueous solution. An “aqueous solution” herein can comprise one or more dissolved salts, where the maximal total salt concentration can be about 3.5 wt % in some embodiments. Although aqueous liquids herein typically comprise water as the only solvent in the liquid, an aqueous liquid can optionally comprise one or more other solvents (e.g., polar organic solvent) that are miscible in water. Thus, an aqueous solution can comprise a solvent having at least about 10 wt % water.

An “aqueous composition” herein has a liquid component that comprises about, or at least about, 10, 20, 30, 40, 50, 60, 70, 80, 90, 95, 99, or 100 wt % water, for example. Examples of aqueous compositions include mixtures, solutions, dispersions (e.g., suspensions, colloidal dispersions) and emulsions, for example. In some embodiments, the pH of an aqueous composition is between ~2 and ~11 (e.g., between ~4 and ~9).

As used herein, the term “colloidal dispersion” refers to a heterogeneous system having a dispersed phase and a dispersion medium, i.e., microscopically dispersed insoluble particles are suspended throughout another substance (e.g., an aqueous composition such as water or aqueous solution). An example of a colloidal dispersion herein is a hydrocolloid. The terms “dispersant” and “dispersion agent” are used interchangeably herein to refer to a material that promotes the formation and/or stabilization of a dispersion. “Dispersing” herein refers to the act of preparing a dispersion of a material in an aqueous liquid. As used herein, the term “latex” (and like terms) refers to a dispersion of one or more types of polymer particles in water or aqueous solution. In some aspects, a latex is an emulsion that comprises dispersed particles. An “emulsion” herein is a dispersion of minute droplets of one liquid in another liquid in which the droplets are not soluble or miscible (e.g., a non-polar substance such as oil or other organic liquid such as an alkane, in a polar liquid such as water or aqueous solution).

A glucan ester derivative in some aspects of the present disclosure can provide stability to a dispersion or emulsion. The “stability” (or the quality of being “stable”) of a dispersion or emulsion herein is, for example, the ability of dispersed particles of a dispersion, or liquid droplets dispersed in another liquid (emulsion), to remain dispersed (e.g., about, or at least about, 70, 75, 80, 85, 90, 95, 96, 97, 98, 99, or 100 wt % of the particles of the dispersion or liquid droplets of the emulsion are in a dispersed state) for a period of about, or at least about, 0.5, 1, 2, 4, 6, 9, 12, 18, 24, 30, or 36 months following initial preparation of the dispersion or emulsion. A stable dispersion or emulsion can resist total creaming, sedimentation, flocculation, and/or coalescence of dispersed/emulsified material.

A glucan ester derivative herein that is “soluble”, “aqueous-soluble”, “water-soluble” (and like terms) herein dissolves (or appreciably dissolves) in water or other aqueous conditions, optionally where the aqueous conditions are further characterized to have a pH of 4-9 (e.g., pH 6-8) and/or temperature of about 1 to 130° C. (e.g., 20-25° C.). In contrast, a glucan ester derivative herein that is “insoluble”, “aqueous-insoluble”, “water-insoluble” and the like does not dissolve under these conditions. In some aspects, less than 1.0 gram (e.g., no detectable amount) of an aqueous-insoluble glucan ester derivative dissolves in 1000 milliliters of such aqueous conditions (e.g., water at 23° C.).

The term “viscosity” as used herein refers to the measure of the extent to which a fluid (aqueous or non-aqueous) resists a force tending to cause it to flow. Various units of viscosity that can be used herein include centipoise (cP, cps) and Pascal-second (Pas), for example. A centipoise is one one-hundredth of a poise; one poise is equal to 0.100 kg·m⁻¹·s⁻¹. Viscosity can be reported as “intrinsic viscosity” (IV, n, units of dL/g) in some aspects; this term refers to a measure of the contribution of a glucan polymer to the viscosity of a liquid (e.g., solution) comprising the glucan polymer. IV measurements herein can be obtained, for example, using any suitable method such as disclosed in U.S. Pat. Appl. Publ. Nos. 2017/0002335, 2017/0002336, or 2018/0340199, or Weaver et al. (J. Appl. Polym. Sci. 35:1631-1637) or Chun and Park (Macromol. Chem. Phys. 195:701-711), which are all incorporated herein by reference. IV can be measured, in part, by dissolving glucan polymer (optionally dissolved at about 100° C. for at least 2, 4, or 8 hours) in DMSO with about 0.9 to 2.5 wt % (e.g., 1, 2, 1-2 wt %) LiCl, for example. IV herein can optionally be used as a relative measure of molecular weight.

The terms “polar organic solvent” and “water-miscible organic solvent” (and like terms) are used interchangeably herein. A polar organic solvent is capable of being dissolved in water or an aqueous solution. Thus, a polar organic solvent does not separate out into a different phase when added to water or an aqueous solution. A polar organic solvent contains carbon and at least one heteroatom (i.e., non-carbon or -hydrogen atom) such as oxygen, nitrogen, sulfur, or phosphorous. This contrasts with non-polar organic solvents, which generally comprise only carbon and hydrogen atoms. A polar organic solvent typically has a dielectric constant greater than about 4. A polar organic solvent contains dipoles due to polar bonds.

The term “aprotic polar organic solvent” (and like terms) herein refers to a polar organic solvent that does not have suitably labile hydrogen atoms that can form hydrogen bonds. An aprotic polar organic solvent does not contain hydrogen atoms bonded to an atom with electronegative character; e.g., there are no O—H, N—H, or S—H bonds.

The term “protic polar organic solvent” (and like terms) herein refers to a polar organic solvent that has one or more suitably labile hydrogen atoms that can form hydrogen bonds. A protic polar organic solvent generally contains hydrogen atoms bonded to an atom with electronegative character; e.g., there are one or more O—H, N—H, and/or S—H bonds.

The term “household care product” and like terms typically refer to products, goods and services relating to the treatment, cleaning, caring and/or conditioning of a home and its contents. The foregoing include, for example, chemicals, compositions, products, or combinations thereof having application in such care.

The terms “fabric”, “textile”, “cloth” and the like are used interchangeably herein to refer to a woven material having a network of natural and/or artificial fibers. Such fibers can be in the form of thread or yarn, for example.

A “fabric care composition” and like terms refer to any composition suitable for treating fabric in some manner. Examples of such a composition include laundry detergents and fabric softeners, which are examples of laundry care compositions.

A “detergent composition” herein typically comprises at least a surfactant (detergent compound) and/or a builder. A “surfactant” herein refers to a substance that tends to reduce the surface tension of a liquid in which the substance is dissolved. A surfactant may act as a detergent, wetting agent, emulsifier, foaming agent, and/or dispersant, for example.

The terms “heavy duty detergent”, “all-purpose detergent” and the like are used interchangeably herein to refer to a detergent useful for regular washing of white and colored textiles at any temperature. The terms “low duty detergent”, “fine fabric detergent” and the like are used interchangeably herein to refer to a detergent useful for the care of delicate fabrics such as viscose, wool, silk, microfiber or other fabric requiring special care. “Special care” can include conditions of using excess water, low agitation, and/or no bleach, for example.

The terms “fabric softener”, “fabric conditioner” and the like herein refer to compositions, such as in liquid or solid form, that deposit lubricants and/or other surface-modifying ingredients onto fabric to, for example, help maintain softness of the fabric and/or provide other beneficial features to fabric (e.g., lubricity, anti-static, anti-cling, and/or anti-wrinkling). A fabric softener herein typically is applied to fabric following fabric washing with a laundry detergent, usually while rinsing the fabric.

The term “personal care product” and like terms typically refer to products, goods and services relating to the treatment, cleaning, cleansing, caring or conditioning of a person. The foregoing include, for example, chemicals, compositions, products, or combinations thereof having application in such care.

An “oral care composition” herein is any composition suitable for treating a soft or hard surface in the oral cavity such as dental (teeth) and/or gum surfaces.

The terms “ingestible product”, “ingestible composition” and the like refer to any substance that, either alone or together with another substance, may be taken orally (i.e., by mouth), whether intended for consumption or not. Thus, an ingestible product includes food/beverage products. “Food/beverage products” refer to any edible product intended for consumption (e.g., for nutritional purposes) by humans or animals, including solids, semi-solids, or liquids. A “food” herein can optionally be referred to as a “foodstuff”, “food product”, or other like term, for example. “Non-edible products” (“non-edible compositions”) refer to any composition that can be taken by the mouth for purposes other than food or beverage consumption. Examples of non-edible products herein include supplements, nutraceuticals, functional food products, pharmaceutical products, oral care products (e.g., dentifrices, mouthwashes), and cosmetic products such as sweetened lip balms. A “pharmaceutical product”, “medicine”, “medication”, “drug” or like term herein refers to a composition used to treat disease or injury, and can be administered enterally or parenterally.

The term “medical product” and like terms typically refer to products, goods and services relating to the diagnosis, treatment, and/or care of patients.

The term “industrial product” and like terms typically refer to products, goods and services used in industrial and/or institutional settings, but typically not by individual consumers.

The terms “flocculant”, “flocculation agent”, “flocculation composition”, “agglomeration agent”, and the like herein refer to substances that can promote agglomeration/clumping/coalescence of insoluble particles suspended in water or other aqueous liquid, thereby rendering the particles more easy to remove by settling/sedimentation, filtration, pelleting, and/or other suitable means. Flocculation of particles typically can be performed in a process of removing/separating particles from an aqueous suspension. Glucan ester derivatives in some aspects can be used as flocculants.

The terms “film”, “sheet” and like terms herein refer to a generally thin, visually continuous material. A film can be comprised as a layer or coating on a material, or can be alone (e.g., not attached to a material surface; free-standing). A “coating” (and like terms) as used herein refers to a layer covering a surface of a material. The term “uniform thickness” as used to characterize a film or coating herein can refer to a contiguous area that (i) is at least 20% of the total film/coating area, and (ii) has a standard deviation of thickness of less than about 50 nm, for example. The term “continuous layer” means a layer of a composition applied to at least a portion of a substrate, wherein a dried layer of the composition covers≥99% of the surface to which it has been applied and having less than 1% voids in the layer that expose the substrate surface. The ≥99% of the surface to which the layer has been applied excludes any area of the substrate to which the layer has not been applied. A coating herein can make a continuous layer in some aspects. A coating composition (and like terms) herein refers to all the solid components that form a layer on a substrate, such as a glucan ester derivative herein and, optionally, pigment, surfactant, dispersing agent, binder, crosslinking agent, and/or other additives.

The terms “fiber”, “fibers” and the like herein refer to staple fibers (staple length fibers) and continuous fibers, in some aspects. Fibers herein can comprise alpha-1,3-glucan, natural fiber (e.g., cellulose, cotton, wool, silk), or synthetic fiber (e.g., polyester), or any other type of material disclosed herein that can form a fiber.

The term “fibrids”, “glucan fibrids”, “fibrillated glucan” and the like as used herein can refer to nongranular, fibrous, or film-like glucan particles with at least one of their three dimensions being of minor magnitude relative to the largest dimension. In some aspects, a glucan fibrid can have a fiber-like and/or a sheet-like structure with a relatively large surface area when compared to a glucan fiber. The surface area of fibrids herein can be, for example, about 5 to 50 meter²/gram of material, with the largest dimension of about 10 to 1000 microns and the smallest dimension of 0.05 to 0.25 microns (aspect ratio of largest to smallest dimension of 40 to 20000).

The terms “non-woven”, “non-woven product”, “non-woven web” and the like herein refer to a web of individual fibers or filaments that are interlaid, typically in a random or unidentifiable manner. This contrasts with a knitted or woven fabric, which has an identifiable network of fibers or filaments. In some aspects, a non-woven product comprises a non-woven web that is bound or attached to another material such as a substrate or backing. A non-woven in some aspects can further contain a binder or adhesive (strengthening agent) that binds adjacent non-woven fibers together. A non-woven binder or adhesive agent can be applied to the non-woven in the form of a dispersion/latex, solution, or solid, for example, and then the treated non-woven is typically dried.

The term “paint” (and like terms) herein is a type of coating composition that is a dispersion of a pigment in a suitable liquid (e.g., aqueous liquid) that can be used to form an adherent coating when spread on a surface in a thin coat. Paint as applied to a surface can provide coloration/decoration, protection, and/or treatment (e.g., primer) to the surface. A paint in some aspects, by virtue of further comprising dispersed particles, can optionally be characterized as a latex or latex paint.

A “composite” herein comprises two or more components including a composition (e.g., glucan ester derivative) of the present disclosure. Typically, the components of a composite resist separation and one or more of the components display enhanced and/or different properties as compared to its properties alone, outside the composite (i.e., a composite is not simply an admixture, which generally is easily separable to its original components). A composite herein generally is a solid material, and can be made via an extrusion or molding process, for example.

The terms “sequence identity”, “identity” and the like as used herein with respect to a polypeptide amino acid sequence (e.g., that of a glucosyltransferase) are as defined and determined in U.S. Patent Appl. Publ. No. 2017/0002336, which is incorporated herein by reference.

Various polypeptide amino acid sequences are disclosed herein as features of certain embodiments. Variants of these sequences that are at least about 70-85%, 85-90%, or 90%-95% identical to the sequences disclosed herein can be used or referenced. Alternatively, a variant amino acid sequence can have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identity with a sequence disclosed herein. The variant amino acid sequence has the same function/activity of the disclosed sequence, or at least about 80%, 81%, 82%, 83%, 84%, 85%, 86%, 87%, 88%, 89%, 90%, 91%, 92%, 93%, 94%, 95%, 96%, 97%, 98%, or 99% of the function/activity of the disclosed sequence.

A composition herein that is “dry” or “dried” typically has less than 6, 5, 4, 3, 2, 1, 0.5, or 0.1 wt % water comprised therein.

The terms “percent by volume”, “volume percent”, “vol %”, “v/v %” and the like are used interchangeably herein. The percent by volume of a solute in a solution can be determined using the formula: [(volume of solute)/(volume of solution)]×100%.

The terms “percent by weight”, “weight percentage (wt %)”, “weight-weight percentage (% w/w)” and the like are used interchangeably herein. Percent by weight refers to the percentage of a material on a mass basis as it is comprised in a composition, mixture, or solution.

The terms “weight/volume percent”, “w/v %” and the like are used interchangeably herein. Weight/volume percent can be calculated as: ((mass [g] of material)/(total volume [mL] of the material plus the liquid in which the material is placed))×100%. The material can be insoluble in the liquid (i.e., be a solid phase in a liquid phase, such as with a dispersion), or soluble in the liquid (i.e., be a solute dissolved in the liquid).

The term “isolated” means a substance (or process) in a form or environment that does not occur in nature. A non-limiting example of an isolated substance includes any glucan ester derivative disclosed herein. It is believed that the embodiments disclosed herein are synthetic/man-made (could not have been made or practiced except for human intervention/involvement), and/or have properties that are not naturally occurring.

The term “increased” as used herein can refer to a quantity or activity that is at least about 1%, 2%, 3%, 4%, 5%, 6%, 7%, 8%, 9%, 10%, 11%, 12%, 13%, 14%, 15%, 16%, 17%, 18%, 19%, 20%, 50%, 100%, or 200% more than the quantity or activity for which the increased quantity or activity is being compared. The terms “increased”, “elevated”, “enhanced”, “greater than”, “improved” and the like are used interchangeably herein.

Some aspects of the present disclosure concern a composition comprising an ester derivative of a glucan, wherein the glucan has a degree of substitution (DoS) up to about 3.0 with at least one cationic organic group (cationic acyl group) that is ester-linked to the glucan. Cationic glucan ester derivatives/compounds are thus disclosed. Yet, some aspects concern a composition comprising an ester derivative of a glucan, wherein the glucan has a DoS up to about 3.0 with at least two organic groups that are individually ester-linked to the glucan, wherein (i) at least one of the organic groups is a cationic organic group, and (ii) at least one of the organic groups is a hydrophobic organic group. By “individually ester linked”, it is meant that the at least two organic groups of (i) and (ii) are each linked to the glucan via its own respective ester linkages. Since a glucan ester derivative can be characterized in some aspects to have at least one cationic ester group and at least one hydrophobic ester group, this type of glucan ester can optionally be characterized as amphiphilic.

A glucan ester derivative herein can be an alpha-glucan ester derivative, for example. The glycosidic linkages of an alpha-glucan ester derivative herein typically are about, or at least about, 80%, 81%, 82%, 83%, 84%, 85%, 86%, 87%, 88%, 89%, 90%, 91%, 92%, 93%, 94%, 95%, 96%, 97%, 98%, 99%, or 100% alpha-glycosidic linkages. Examples of suitable alpha-glucan ester derivatives include ester derivatives of alpha-1,3-glucan, alpha-1,6-glucan, and alpha-1,4-glucan.

In some aspects, an alpha-glucan ester comprises about, or at least about, 50%, 60%, 70%, 80%, 85%, 86%, 87%, 88%, 89%, 90%, 91%, 92%, 93%, 94%, 95%, 96%, 97%, 98%, 99%, 99.5%, or 100% alpha-1,3 glycosidic linkages (i.e., the ester is an alpha-1,3-glucan ester). In some aspects, accordingly, an alpha-glucan ester has about, or less than about, 50%, 40%, 30%, 20%, 15%, 10%, 9%, 8%, 7%, 6%, 5%, 4%, 3%, 2%, 1%, 0.5%, or 0% glycosidic linkages that are not alpha-1,3. Typically, the glycosidic linkages that are not alpha-1,3 are mostly or entirely alpha-1,6. In some aspects, an alpha-glucan ester has no branch points or less than about 5%, 4%, 3%, 2%, or 1% branch points as a percent of the glycosidic linkages in the alpha-glucan.

The DPw, DPn, or DP of the alpha-glucan portion of an alpha-1,3-glucan ester in some aspects can be about, at least about, or less than about, 2, 3, 4, 5, 6, 7, 8, 9, 10, 15, 25, 50, 75, 100, 150, 200, 300, 400, 500, 600, 700, 800, 900, 1000, 1100, 1200, 1300, 1400, 1500, 1600, 1700, 1800, 1900, 2000, 2500, 3000, 3500, or 4000. DPw, DPn, or DP can optionally be expressed as a range between any two of these values. Merely as examples, the DPw, DPn, or DP can be about 50-1600, 100-1600, 200-1600, 300-1600, 400-1600, 500-1600, 600-1600, 700-1600, 50-1250, 100-1250, 200-1250, 300-1250, 400-1250, 500-1250, 600-1250, 700-1250, 50-1000, 100-1000, 200-1000, 300-1000, 400-1000, 500-1000, 600-1000, 700-1000, 50-900, 100-900, 200-900, 300-900, 400-900, 500-900, 600-900, 700-900, 600-800, or 600-750. Merely as further examples, the DPw, DPn, or DP can be about 15-100, 25-100, 35-100, 15-80, 25-80, 35-80, 15-60, 25-60, 35-60, 15-55, 25-55, 35-55, 15-50, 25-50, 35-50, 35-45, 35-40, 40-100, 40-80, 40-60, 40-55, 40-50, 45-60, 45-55, 45-50, 15-35, 20-35, 15-30, or 20-30. Merely as further examples, the DPw, DPn, or DP can be about 100-600, 100-500, 100-400, 100-300, 200-600, 200-500, 200-400, or 200-300. In some aspects, the alpha-glucan portion of an alpha-1,3-glucan ester can have a high molecular weight as reflected by high intrinsic viscosity (IV); e.g., IV can be about, or at least about, 6, 7, 8, 9, 10, 11, 12, 13, 14, 15, 16, 17, 18, 19, 20, 21, 22, 6-8, 6-7, 6-22, 6-20, 6-17, 6-15, 6-12, 10-22, 10-20, 10-17, 10-15, 10-12, 12-22, 12-20, 12-17, or 12-15 dL/g (for comparison purposes, note that the IV of alpha-glucan with at least 90% (e.g., about 99% or 100%) alpha-1,3 linkages and a DPw of about 800 has an IV of about 2-2.5 dL/g). IV herein can be as measured with alpha-glucan polymer dissolved in DMSO with about 0.9 to 2.5 wt % (e.g., 1, 2, 1-2 wt %) LiCl, for example. The molecular weight of an alpha-1,3-glucan ester herein can be calculated, for example, based on any of the foregoing alpha-1,3-glucan DPw, DPn, or DP values, further taking into account the ester's DoS and type of ester group(s); such molecular weight can be about, at least about, or less than about, the calculated value (this mode of molecular weight calculation can be applied to any other polysaccharide/glucan ester derivative disclosed herein).

The alpha-1,3-glucan portion of an alpha-1,3-glucan ester derivative herein can be as disclosed (e.g., molecular weight, linkage profile, and/or production method), for example, in U.S. Pat. Nos. 7,000,000, 8,871,474, 10,301,604, or 10,260,053, or U.S. Patent Appl. Publ. Nos. 2019/0112456, 2019/0078062, 2019/0078063, 2018/0340199, 2018/0021238, 2018/0273731, 2017/0002335, 2015/0232819, 2015/0064748, 2020/0165360, 2020/0131281, or 2019/0185893, which are each incorporated herein by reference. Alpha-1,3-glucan can be produced, for example, by an enzymatic reaction comprising at least water, sucrose and a glucosyltransferase enzyme that synthesizes the alpha-1,3-glucan. Glucosyltransferases, reaction conditions, and/or processes contemplated to be useful for producing insoluble alpha-glucan can be as disclosed in any of the foregoing references.

In some aspects, a glucosyltransferase enzyme for producing an alpha-1,3-glucan portion of an alpha-1,3-glucan ester derivative herein can comprise an amino acid sequence that is 100% identical to, or at least 90%, 91%, 92%, 93%, 94%, 95%, 96%, 97%, 98%, 98.5%, 99%, or 99.5% identical to, SEQ ID NO:2, 4, 6, 8, 10, 12, 14, 16, 18, 20, 26, 28, 30, 34, or 59, or amino acid residues 55-960 of SEQ ID NO:4, residues 54-957 of SEQ ID NO: 65, residues 55-960 of SEQ ID NO:30, residues 55-960 of SEQ ID NO:28, or residues 55-960 of SEQ ID NO:20, and have glucosyltransferase activity; these amino acid sequences are disclosed in U.S. Patent Appl. Publ. No. 2019/0078063, which is incorporated herein by reference. It is noted that a glucosyltransferase enzyme comprising SEQ ID NO:2, 4, 8, 10, 14, 20, 26, 28, 30, 34, or amino acid residues 55-960 of SEQ ID NO: 4, residues 54-957 of SEQ ID NO:65, residues 55-960 of SEQ ID NO:30, residues 55-960 of SEQ ID NO:28, or residues 55-960 of SEQ ID NO:20, can synthesize insoluble alpha-glucan comprising at least about 90% (~100%) alpha-1,3 linkages.

In some aspects, an alpha-glucan ester comprises about, or at least about, 50%, 60%, 70%, 80%, 85%, 90%, 91%, 92%, 93%, 94%, 95%, 96%, 97%, 98%, 99%, 99.5%, or 100% alpha-1,6 glycosidic linkages (i.e., the ester is an alpha-1,6-glucan ester, or dextran ester). In some aspects, a substantially linear dextran ester can comprise 5%, 4%, 3%, 2%, 1%, 0.5% or less glycosidic branches (a linear dextran ester has 100% alpha-1,6 linkages). If present, glycosidic branches from a dextran ester are typically short, being one (pendant), two, or three glucose monomers in length. In some aspects, a dextran ester can comprise about, or less than about, 50%, 40%, 30%, 20%, 15%, 10%, 9%, 8%, 7%, 6%, 5%, 4%, 3%, 2%, 1%, 0.5%, or 0% alpha-1,4, alpha-1,3 and/or alpha-1,2 glycosidic linkages. Typically, such linkages exist entirely, or almost entirely, as branch points from alpha-1,6-glucan.

The dextran portion of a dextran ester derivative herein can have alpha-1,2, alpha-1,3, and/or alpha-1,4 branches, for example. In some aspects, about, at least about, or less than about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 2-25%, 2-20%, 2-15%, 2-10%, 5-25%, 5-20%, 5-15%, 5-10%, 7-13%, 8-12%, 9-11%, 10-25%, 10-20%, 10-15%, 10-22%, 12-20%, 12-18%, 14-20%, 14-18%, 15-18%, or 15-17% of all the glycosidic linkages of a branched dextran ester are alpha-1,2, alpha-1,3, and/or alpha-1,4 glycosidic branch linkages. Such branches typically are mostly (>90% or >95%), or all (100%), a single glucose monomer in length. In some aspects, dextran with alpha-1,2-branching can be produced enzymatically according to the procedures in U.S. Patent Appl. Publ. Nos. 2017/0218093 or 2018/0282385 (both incorporated herein by reference) where, for example, an alpha-1,2-branching enzyme such as GTFJ18T1 or GTF9905 can be added during or after the production of the dextran. In some aspects, any other enzyme known to produce alpha-1,2-branching can be used. Dextran with alpha-1,3-branching can be prepared, for example, as disclosed in Vuillemin et al. (2016, J. Biol Chem. 291:7687-7702) or Int. Patent Appl. Publ. No. WO2021/007264, which are incorporated herein by reference.

The dextran portion of a dextran ester derivative herein can have a DPw, DPn, or DP of about, at least about, or less than about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 85, 90, 95, 100, 105, 110, 150, 200, 250, 300, 400, 500, 1000, 1500, 2000, 2500, 3000, 4000, 5000, 6000, 8-20, 8-30, 8-100, 8-500, 3-4, 3-5, 3-6, 3-7, 3-8, 4-5, 4-6, 4-7, 4-8, 5-6, 5-7, 5-8, 6-7, 6-8, 7-8, 90-120, 95-120, 100-120, 105-120, 110-120, 115-120, 90-115, 95-115, 100-115, 105-115, 110-115, 90-110, 95-110, 100-110, 105-110, 90-105, 95-105, 100-105, 90-100, 95-100, 90-95, 85-95, 85-90, 5-100, 5-250, 5-500, 5-1000, 5-1500, 5-2000, 5-2500, 5-3000, 5-4000, 5-5000, 5-6000, 10-100, 10-250, 10-500, 10-1000, 10-1500, 10-2000, 10-2500, 10-3000, 10-4000, 10-5000, 10-6000, 25-100, 25-250, 25-500, 25-1000, 25-1500, 25-2000, 25-2500, 25-3000, 25-4000, 25-5000, 25-6000, 50-100, 50-250, 50-500, 50-1000, 50-1500, 50-2000, 50-2500, 50-3000, 50-4000, 50-5000, 50-6000, 100-100, 100-250, 100-400, 100-500, 100-1000, 100-1500, 100-2000, 100-2500, 100-3000, 100-4000, 100-5000, 100-6000, 250-500, 250-1000, 250-1500, 250-2000, 250-2500, 250-3000, 250-4000, 250-5000, 250-6000, 300-2800, 300-3000, 350-2800, 350-3000, 500-1000, 500-1500, 500-2000, 500-2500, 500-2800, 500-3000, 500-4000, 500-5000, 500-6000, 600-1550, 600-1850, 600-2000, 600-2500, 600-3000, 750-1000, 750-1250, 750-1500, 750-2000, 750-2500, 750-3000, 750-4000, 750-5000, 750-6000, 900-1250, 900-1500, 900-2000, 1000-1250, 1000-1400, 1000-1500, 1000-2000, 1000-2500, 1000-3000, 1000-4000, 1000-5000, 1000-6000, or 1100-1300, for example. The molecular weight (e.g., Mw or Mn) of the dextran portion of a dextran ester derivative in some aspects can be about, at least about, or less than about, 0.1, 0.125, 0.15, 0.175, 0.2, 0.24, 0.25, 0.5, 0.75, 1, 2, 3, 4, 5, 6, 7, 8, 9, 10, 20, 30, 40, 50, 60, 70, 80, 90, 100, 110, 120, 130, 140, 150, 160, 170, 180, 190, 200, 0.1-0.2, 0.125-0.175, 0.13-0.17, 0.135-0.165, 0.14-0.16, 0.145-0.155, 10-80, 20-70, 30-60, 40-50, 50-200, 60-200, 70-200, 80-200, 90-200, 100-200, 110-200, 120-200, 50-180, 60-180, 70-180, 80-180, 90-180, 100-180, 110-180, 120-180, 50-160, 60-160, 70-160, 80-160, 90-160, 100-160, 110-160, 120-160, 50-140, 60-140, 70-140, 80-140, 90-140, 100-140, 110-140, 120-140, 50-120, 60-120, 70-120, 80-120, 90-120, 90-110, 100-120, 110-120, 50-110, 60-110, 70-110, 80-110, 90-110, 100-110, 50-100, 60-100, 70-100, 80-100, 90-100, or 95-105 million Daltons. The molecular weight (e.g., Mw or Mn) of the dextran portion of a dextran ester derivative in some aspects can be about, at least about, or less than about, 1, 5, 7.5, 10, 12.5, 15, 20, 30, 40, 50, 60, 70, 80, 90, 100, 150, 200, 250, 300, 400, 500, 600, 700, 800, 900, 1000, 1250, 1500, 1750, 2000, 1-2000, 1-1000, 1-500, 1-400, 1-300, 1-200, 1-100, 1-50, 10-2000, 10-1000, 10-500, 10-400, 10-300, 10-200, 10-100, 10-50, 20-2000, 20-1000, 20-500, 20-400, 20-300, 20-200, 20-100, 20-50, 30-2000, 30-1000, 30-500, 30-400, 30-300, 30-200, 30-100, 30-50, 40-2000, 40-1000, 40-500, 40-400, 40-300, 40-200, 40-100, 40-50, 50-2000, 50-1000, 50-500, 50-400, 50-300, 50-200, 100-2000, 100-1000, 100-500, 100-400, 100-300, 100-200, 200-2000, 20-1000, 200-500, 200-400, 200-300, 7.5-10, 7.5-12.5, 7.5-15, 7.5-20, 7.5-30, 10-12.5, 10-15, 10-20, 10-30, 15-25, 15-30, 40-60, 45-55, 190-210, or 290-310 kDa, for example. The molecular weight of a dextran ester herein can be calculated, for example, based on any of the foregoing dextran DPw, DPn, DP, or Dalton values, further taking into account the ester's DoS and type of ester group(s); such a molecular weight can be about, at least about, or less than about, any of above molecular weight values or ranges. Any of the forgoing DPw, DPn, DP, or Dalton values can characterize a dextran herein before, or after, it has optionally been branched (e.g., alpha-1,2 and/or alpha-1,3), for instance.

The dextran portion of a dextran ester derivative herein can be as disclosed (e.g., molecular weight, linkage/branching profile, production method), for example, in U.S. Patent Appl. Publ. Nos. 2016/0122445, 2017/0218093, 2018/0282385, 2020/0165360, or 2019/0185893, which are each incorporated herein by reference. In some aspects, a dextran for ester derivatization herein can be one produced in a suitable reaction comprising glucosyltransferase (GTF) 0768 (SEQ ID NO:1 or 2 of US2016/0122445), GTF 8117, GTF 6831, or GTF 5604 (these latter three GTF enzymes are SEQ ID NOs: 30, 32 and 33, respectively, of US2018/0282385), or a GTF comprising an amino acid sequence that is at least 90%, 91%, 92%, 93%, 94%, 95%, 96%, 97%, 98%, or 99% identical to the amino acid sequence of GTF 0768, GTF 8117, GTF 6831, or GTF 5604.

In some aspects, the alpha-glucan portion of an alpha-glucan ester derivative can be in the form of a graft copolymer such as disclosed in U.S. Patent Appl. Publ. Nos. 2020/0165360, 2019/0185893, or 2020/0131281, which are incorporated herein by reference. A graft copolymer can comprise dextran (as backbone) and alpha-1,3-glucan (as one or more side chains), where the latter component has been grafted onto the former component; typically, this graft copolymer is produced by using dextran or alpha-1,2- and/or alpha-1,3-branched dextran as a primer for alpha-1,3-glucan synthesis by an alpha-1,3-glucan-producing glucosyltransferase as described above. Alpha-1,3-glucan side chain(s) of an alpha-glucan graft copolymer herein can be alpha-1,3-glucan as presently disclosed. Dextran backbone of an alpha-glucan graft copolymer herein can be dextran or alpha-1,2- and/or alpha-1,3-branched dextran as presently disclosed. In some aspects, an alpha-glucan graft copolymer can comprise: (A) an alpha-1,6-glucan backbone (100% alpha-1,6-linked before alpha-1,2 and/or alpha-1,3 branching) that (i) has been branched with about 10-22% (e.g., about 12-20%, 12-18%, 14-20%, 14-18%, 15-18%, 15-17%, or 16%) alpha-1,2 and/or alpha-1,3 linkages (i.e., alpha-1,2,6 and/or alpha-1,3,6) (e.g., the backbone in total comprises about 82-86% or 84% alpha-1,6 linkages and about 14-18% or 16% alpha-1,2 and/or alpha-1,3 linkages) and (ii) has an Mw of about 15-25, 15-22.5, 17-25, 17-22.5, 18-22, or 20 kDa, and (B) one or more (e.g., two, three, four, five, or six) alpha-1,3-glucan side chains that have been extended from one or more of the alpha-1,2 and/or alpha-1,3 branches; such a graft copolymer typically is water-insoluble.

In some aspects, an alpha-glucan ester comprises about, or at least about, 50%, 60%, 70%, 80%, 85%, 90%, 91%, 92%, 93%, 94%, 95%, 96%, 97%, 98%, 99%, 99.5%, or 100% alpha-1,4 glycosidic linkages (i.e., the ester is an alpha-1,4-glucan ester). In some aspects, accordingly, an alpha-1,4-glucan ester has about, or less than about, 50%, 40%, 30%, 20%, 15%, 10%, 9%, 8%, 7%, 6%, 5%, 4%, 3%, 2%, 1%, 0.5%, or 0% glycosidic linkages that are not alpha-1,4. Examples of alpha-1,4-glucan herein include amylose, amylopectin, and starch. Alpha-1,4-glucan such as starch can be derived from vegetable (e.g., potato, tapioca, peas, palm) or grain (e.g., corn, wheat, rice, barley) sources, for example.

The DPw, DPn, or DP of the alpha-1,4-glucan portion of an alpha-1,4-glucan ester derivative in some aspects can be about, at least about, or less than about, 10, 25, 50, 75, 100, 150, 200, 300, 400, 500, 600, 700, 800, 900, 1000, 1100, 1200, 1300, 1400, 1500, 1600, 1700, 1800, 1900, 2000, 2500, 3000, 3500, or 4000. DPw, DPn, or DP can optionally be expressed as a range between any two of these values. In some aspects, the DPW, DPn, or DP of the alpha-1,4-glucan portion of an alpha-1,4-glucan ester derivative can be as disclosed above for alpha-1,3-glucan or alpha-1,6-glucan.

A glucan ester derivative herein can be a beta-glucan ester derivative, for example. The glycosidic linkages of a beta-glucan ester derivative herein typically are about, or at least about, 80%, 81%, 82%, 83%, 84%, 85%, 86%, 87%, 88%, 89%, 90%, 91%, 92%, 93%, 94%, 95%, 96%, 97%, 98%, 99%, or 100% beta-glycosidic linkages. Examples of suitable beta-glucan ester derivatives include ester derivatives of beta-1,3-glucan (e.g., laminarin, paramylon, curdlan), beta-1,4-glucan (cellulose), and beta-1,6-glucan. In some aspects, a glucan ester herein is not a beta-glucan ester, and/or does not comprise a beta-glycosidic linkage.

In some aspects, a beta-glucan ester comprises about, or at least about, 80%, 81%, 82%, 83%, 84%, 85%, 86%, 87%, 88%, 89%, 90%, 91%, 92%, 93%, 94%, 95%, 96%, 97%, 98%, 99%, 99.5%, or 100% beta-1,4 glycosidic linkages (i.e., the ester is a beta-1,4-glucan ester). The DPw, DPn, or DP of the beta-1,4-glucan portion of a beta-1,4-glucan ester derivative in some aspects can be about, or at least about, 10, 15, 20, 25, 30, 35, 40, 45, 50, 75, 100, 150, 200, 300, 400, 500, 600, 700, 800, 900, 1000, 1100, 1200, 1300, 1400, 1500, 1600, 1700, 1800, 1900, 2000, 2500, 3000, 3500, or 4000. The DPw, DPn, or DP can optionally be expressed as a range between any two of these values (e.g., 1000-2000, 1300-1700, 1400-1600). In some aspects, the DPw, DPn, or DP of the beta-1,4-glucan portion of a beta-1,4-glucan ester derivative can be as disclosed above for alpha-1,3-glucan or alpha-1,6-glucan. In some aspects, a glucan ester herein is not a beta-1,4-glucan ester, and/or does not comprise a beta-1,4 glycosidic linkage.

In some aspects, a beta-glucan ester comprises about, or at least about, 80%, 81%, 82%, 83%, 84%, 85%, 86%, 87%, 88%, 89%, 90%, 91%, 92%, 93%, 94%, 95%, 96%, 97%, 98%, 99%, 99.5%, or 100% beta-1,3 glycosidic linkages (i.e., the ester is a beta-1,3-glucan ester). The DPw, DPn, or DP of the beta-1,3-glucan portion of a beta-1,3-glucan ester derivative in some aspects can be about, at least about, or less than about, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 150, 200, 300, 400, 500, 600, 700, 800, 900, 1000, 1100, 1200, 1300, 1400, 1500, 1600, 1700, 1800, 1900, 2000, 2500, 3000, 3500, 4000, 3-15, 3-20, 3-25, 3-30, 5-15, 5-20, 5-25, 5-30, 10-15, 10-20, 10-25, 10-30, 15-17, 15-18, 15-19, 15-20, 15-21, 15-22, 15-23, 15-24, 15-25, 15-30, 16-17, 16-18, 16-19, 16-20, 16-21, 16-22, 16-23, 16-24, 16-25, 16-30, 17-18, 17-19, 17-20, 17-21, 17-22, 17-23, 17-24, 17-25, 17-30, 20-25, 20-30, or 25-30, for example. In some aspects, the DPw, DPn, or DP of the beta-1,3-glucan portion of a beta-1,3-glucan ester derivative can be as disclosed above for alpha-1,3-glucan or alpha-1,6-glucan.

In some additional or alternative aspects herein, an ester derivative can be a soy polysaccharide ester derivative. The soy polysaccharide portion of a soy polysaccharide ester derivative in some aspects can be as disclosed in U.S. Pat. Appl. Publ. No. 2018/0079832, which is incorporated herein by reference. Accordingly, any of the features of the present disclosure regarding a glucan ester derivative can likewise characterize embodiments in which a soy polysaccharide ester derivative is used, insofar as would be considered suitable by a skilled artisan. For example, insofar as would be considered suitable by a skilled artisan, the term “glucan ester derivative” (and like terms) as used in the present disclosure can optionally be replaced with the term “soy polysaccharide ester derivative”.

An ester derivative of a polysaccharide/glucan in some aspects of the present disclosure can have a degree of substitution (DoS) up to about 3.0 (e.g., 0.001 to 3.0) with at least two organic groups that are individually ester-linked to the glucan, wherein (i) at least one of the organic groups is a cationic organic group (cationic acyl group), and (ii) at least one of the organic groups is a hydrophobic organic group (hydrophobic acyl group). Yet, in some aspects, an ester derivative of a polysaccharide/glucan can have a DoS up to about 3.0 (e.g., 0.001 to 3.0) with at least one cationic organic group (cationic acyl group) that is ester-linked to the polysaccharide/glucan.

The DoS (or, optionally, “total DoS” in this aspect) of a glucan herein with at least one cationic organic group and at least one hydrophobic organic group can be about, at least about, or up to about, 0.001, 0.0025, 0.005, 0.01, 0.02, 0.025, 0.03, 0.04, 0.05, 0.06, 0.07, 0.075, 0.08, 0.09, 0.1, 0.15, 0.2, 0.25, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 (DoS can optionally be expressed as a range between any two of these values), for example. Some examples of DoS ranges herein include 0.005-2.0, 0.005-1.9, 0.005-1.8, 0.005-1.7, 0.005-1.6, 0.005-1.5, 0.005-1.25, 0.005-1.0, 0.005-0.9, 0.005-0.8, 0.005-0.7, 0.005-0.6, 0.005-0.5, 0.01-2.0, 0.01-1.9, 0.01-1.8, 0.01-1.7, 0.01-1.6, 0.01-1.5, 0.01-1.25, 0.01-1.0, 0.01-0.9, 0.01-0.8, 0.01-0.7, 0.01-0.6, 0.01-0.5, 0.01-0.25, 0.01-0.1, 0.03-2.0, 0.03-1.9, 0.03-1.8, 0.03-1.7, 0.03-1.6, 0.03-1.5, 0.03-1.25, 0.03-1.0, 0.03-0.9, 0.03-0.8, 0.03-0.7, 0.03-0.6, 0.03-0.5, 0.03-0.25, 0.03-0.1, 0.05-2.0, 0.05-1.9, 0.05-1.8, 0.05-1.7, 0.05-1.6, 0.05-1.5, 0.05-1.25, 0.05-1.0, 0.05-0.9, 0.05-0.8, 0.05-0.7, 0.05-0.6, 0.05-0.5, 0.1-2.0, 0.1-1.9, 0.1-1.8, 0.1-1.7, 0.1-1.6, 0.1-1.5, 0.1-1.25, 0.1-1.0, 0.1-0.9, 0.1-0.8, 0.1-0.7, 0.1-0.6, 0.1-0.5, 0.15-2.0, 0.15-1.9, 0.15-1.8, 0.15-1.7, 0.15-1.6, 0.15-1.5, 0.15-1.25, 0.15-1.0, 0.15-0.9, 0.15-0.8, 0.15-0.7, 0.15-0.6, 0.15-0.5, 0.2-2.0, 0.2-1.9, 0.2-1.8, 0.2-1.7, 0.2-1.6, 0.2-1.5, 0.2-1.25, 0.2-1.0, 0.2-0.9, 0.2-0.8, 0.2-0.7, 0.2-0.6, 0.2-0.5, 0.25-2.0, 0.25-1.9, 0.25-1.8, 0.25-1.7, 0.25-1.6, 0.25-1.5, 0.25-1.25, 0.25-1.0, 0.25-0.9, 0.25-0.8, 0.25-0.7, 0.25-0.6, 0.25-0.5, 0.3-2.0, 0.3-1.9, 0.3-1.8, 0.3-1.7, 0.3-1.6, 0.3-1.5, 0.3-1.25, 0.3-1.0, 0.3-0.9, 0.3-0.8, 0.3-0.7, 0.3-0.6, 0.3-0.5, 0.4-2.0, 0.4-1.9, 0.4-1.8, 0.4-1.7, 0.4-1.6, 0.4-1.5, 0.4-1.25, 0.4-1.0, 0.4-0.9, 0.4-0.8, 0.4-0.7, 0.4-0.6 and 0.4-0.5.

The DoS of a glucan herein with at least one cationic organic group can be about, at least about, or up to about, 0.001, 0.0025, 0.005, 0.01, 0.02, 0.025, 0.03, 0.04, 0.05, 0.06, 0.07, 0.075, 0.08, 0.09, 0.1, 0.15, 0.2, 0.25, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 (DoS can optionally be expressed as a range between any two of these values), for example. Some examples of DoS ranges herein include 0.005-2.0, 0.005-1.9, 0.005-1.8, 0.005-1.7, 0.005-1.6, 0.005-1.5, 0.005-1.25, 0.005-1.0, 0.005-0.9, 0.005-0.8, 0.005-0.7, 0.005-0.6, 0.005-0.5, 0.01-2.0, 0.01-1.9, 0.01-1.8, 0.01-1.7, 0.01-1.6, 0.01-1.5, 0.01-1.25, 0.01-1.0, 0.01-0.9, 0.01-0.8, 0.01-0.7, 0.01-0.6, 0.01-0.5, 0.01-0.25, 0.01-0.1, 0.03-2.0, 0.03-1.9, 0.03-1.8, 0.03-1.7, 0.03-1.6, 0.03-1.5, 0.03-1.25, 0.03-1.0, 0.03-0.9, 0.03-0.8, 0.03-0.7, 0.03-0.6, 0.03-0.5, 0.03-0.25, 0.03-0.1, 0.05-2.0, 0.05-1.9, 0.05-1.8, 0.05-1.7, 0.05-1.6, 0.05-1.5, 0.05-1.25, 0.05-1.0, 0.05-0.9, 0.05-0.8, 0.05-0.7, 0.05-0.6, 0.05-0.5, 0.1-2.0, 0.1-1.9, 0.1-1.8, 0.1-1.7, 0.1-1.6, 0.1-1.5, 0.1-1.25, 0.1-1.0, 0.1-0.9, 0.1-0.8, 0.1-0.7, 0.1-0.6, 0.1-0.5, 0.15-2.0, 0.15-1.9, 0.15-1.8, 0.15-1.7, 0.15-1.6, 0.15-1.5, 0.15-1.25, 0.15-1.0, 0.15-0.9, 0.15-0.8, 0.15-0.7, 0.15-0.6, 0.15-0.5, 0.2-2.0, 0.2-1.9, 0.2-1.8, 0.2-1.7, 0.2-1.6, 0.2-1.5, 0.2-1.25, 0.2-1.0, 0.2-0.9, 0.2-0.8, 0.2-0.7, 0.2-0.6, 0.2-0.5, 0.25-2.0, 0.25-1.9, 0.25-1.8, 0.25-1.7, 0.25-1.6, 0.25-1.5, 0.25-1.25, 0.25-1.0, 0.25-0.9, 0.25-0.8, 0.25-0.7, 0.25-0.6, 0.25-0.5, 0.3-2.0, 0.3-1.9, 0.3-1.8, 0.3-1.7, 0.3-1.6, 0.3-1.5, 0.3-1.25, 0.3-1.0, 0.3-0.9, 0.3-0.8, 0.3-0.7, 0.3-0.6, 0.3-0.5, 0.4-2.0, 0.4-1.9, 0.4-1.8, 0.4-1.7, 0.4-1.6, 0.4-1.5, 0.4-1.25, 0.4-1.0, 0.4-0.9, 0.4-0.8, 0.4-0.7, 0.4-0.6 and 0.4-0.5.

The DoS of a glucan herein with at least one cationic organic group can be about, at least about, or up to about, 0.001, 0.0025, 0.005, 0.01, 0.02, 0.025, 0.03, 0.04, 0.05, 0.06, 0.07, 0.075, 0.08, 0.09, 0.1, 0.15, 0.2, 0.25, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 (DoS can optionally be expressed as a range between any two of these values), for example. Some examples of DoS ranges herein include 0.005-2.0, 0.005-1.9, 0.005-1.8, 0.005-1.7, 0.005-1.6, 0.005-1.5, 0.005-1.25, 0.005-1.0, 0.005-0.9, 0.005-0.8, 0.005-0.7, 0.005-0.6, 0.005-0.5, 0.01-2.0, 0.01-1.9, 0.01-1.8, 0.01-1.7, 0.01-1.6, 0.01-1.5, 0.01-1.25, 0.01-1.0, 0.01-0.9, 0.01-0.8, 0.01-0.7, 0.01-0.6, 0.01-0.5, 0.01-0.25, 0.01-0.1, 0.03-2.0, 0.03-1.9, 0.03-1.8, 0.03-1.7, 0.03-1.6, 0.03-1.5, 0.03-1.25, 0.03-1.0, 0.03-0.9, 0.03-0.8, 0.03-0.7, 0.03-0.6, 0.03-0.5, 0.03-0.25, 0.03-0.1, 0.05-2.0, 0.05-1.9, 0.05-1.8, 0.05-1.7, 0.05-1.6, 0.05-1.5, 0.05-1.25, 0.05-1.0, 0.05-0.9, 0.05-0.8, 0.05-0.7, 0.05-0.6, 0.05-0.5, 0.1-2.0, 0.1-1.9, 0.1-1.8, 0.1-1.7, 0.1-1.6, 0.1-1.5, 0.1-1.25, 0.1-1.0, 0.1-0.9, 0.1-0.8, 0.1-0.7, 0.1-0.6, 0.1-0.5, 0.15-2.0, 0.15-1.9, 0.15-1.8, 0.15-1.7, 0.15-1.6, 0.15-1.5, 0.15-1.25, 0.15-1.0, 0.15-0.9, 0.15-0.8, 0.15-0.7, 0.15-0.6, 0.15-0.5, 0.2-2.0, 0.2-1.9, 0.2-1.8, 0.2-1.7, 0.2-1.6, 0.2-1.5, 0.2-1.25, 0.2-1.0, 0.2-0.9, 0.2-0.8, 0.2-0.7, 0.2-0.6, 0.2-0.5, 0.25-2.0, 0.25-1.9, 0.25-1.8, 0.25-1.7, 0.25-1.6, 0.25-1.5, 0.25-1.25, 0.25-1.0, 0.25-0.9, 0.25-0.8, 0.25-0.7, 0.25-0.6, 0.25-0.5, 0.3-2.0, 0.3-1.9, 0.3-1.8, 0.3-1.7, 0.3-1.6, 0.3-1.5, 0.3-1.25, 0.3-1.0, 0.3-0.9, 0.3-0.8, 0.3-0.7, 0.3-0.6, 0.3-0.5, 0.4-2.0, 0.4-1.9, 0.4-1.8, 0.4-1.7, 0.4-1.6, 0.4-1.5, 0.4-1.25, 0.4-1.0, 0.4-0.9, 0.4-0.8, 0.4-0.7, 0.4-0.6 and 0.4-0.5.

Any of the foregoing DoS values and/or ranges for (i) at least one cationic group, and (ii) at least one hydrophobic group, can be combined, as appropriate, to characterize the DoS profile of a glucan ester derivative herein, for example. In some aspects, any of the foregoing values and/or ranges for total DoS can be combined, as appropriate, with any of the foregoing DoS values and/or ranges for (i) at least one cationic group and/or (ii) at least one hydrophobic group.

Regarding polysaccharide ester derivatives herein that are glucan derivatives, for example, since there are at most three hydroxyl groups in a glucose monomeric unit of a glucan, the overall DoS of a glucan ester derivative can be no higher than 3.0. It would be understood by those skilled in the art that, since a glucan ester derivative as presently disclosed has a DoS with at least one type of organic group (acyl group) in ester linkage (e.g., at least two types of organic group in ester linkage, wherein at least one group is a cationic group and at least one group is a hydrophobic group) (e.g., between about 0.001 to about 3.0), all the substituents of a glucan ester derivative cannot only be hydroxyl.

An ester derivative of a polysaccharide/glucan of the present disclosure can be substituted with at least one cationic organic group (cationic acyl group) herein that is ester-linked to the polysaccharide/glucan. A glucan derivative as presently disclosed can be derivatized with one, two, three, or more different types of esterified cationic organic groups herein, for example. In some aspects, at least one ester-linked cationic organic group comprises Structure I:

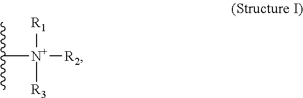

wherein each of R₁, R₂ and R₃ is independently a group comprising at least one carbon atom. The positioning of R₁, R₂ and R₃ in Structure I is generally of no particular importance and not intended to invoke any particular stereochemistry.

Regarding the squiggly line (variable section) of Structure I, since a cationic organic group is ester-linked to a glucan derivative herein, and thus is a cationic acyl group, it would be understood that the —N⁺R₁R₂R₃ portion of Structure I is bound via one or more (chain of) carbon atoms to a glucose monomer unit of the glucan derivative via a carbonyl (—CO—). Such one or more (chain of) carbon atoms can be referred to herein as —R_{c}—. The carbonyl links the —R_{c}—N⁺R₁R₂R₃ portion of the organic group to an oxygen atom of a now-substituted hydroxyl group (i.e., hydrogen atom now replaced by acyl group). Thus, Structure I can optionally be described as —CO—R_{c}—N⁺R₁R₂R₃. As bound to a glucose monomer unit of a glucan, it can be described as —C_{G}—O_{G}—CO—R_{c}—N⁺R₁R₂R₃, where —C_{G}— represents a glucose monomer unit carbon atom and —O_{G}— represents the oxygen atom of a now-substituted glucose unit hydroxyl group.

In some aspects, R_{c} (above) comprises one (e.g., —CH₂—), two (e.g., —CH₂CH₂—), three (e.g., —CH₂CH₂CH₂—), four (e.g., —CH₂CH₂CH₂CH₂—), five, six, seven, eight, nine, ten, eleven, twelve, thirteen, fourteen, fifteen, sixteen, seventeen, eighteen, nineteen, twenty, twenty-one, twenty-two, or more carbon atoms. R_{c} can be completely or partially saturated, for example. R_{c} can be linear, for example. Structure I can be described as —CO—CH₂—N⁺R₁R₂R₃, —CO—CH₂CH₂—N⁺R₁R₂R₃, —CO—CH₂CH₂CH₂—N⁺R₁R₂R₃, or —CO—CH₂CH₂CH₂CH₂—N⁺R₁R₂R₃, for instance.

R_{c} can have one or more substitutions (hydrogen atom replaced by another group) with a hydroxyl group, for example. R_{c} in some aspects can comprise —CH₂CH(OH)—; Structure I comprising such an R_{c} can be described as —CO—CH₂CH(OH)—CH₂—N⁺R₁R₂R₃, —CO—CH₂CH(OH)—CH₂CH₂—N⁺R₁R₂R₃, —CO—CH₂CH(OH)—CH₂CH₂CH₂—N⁺R₁R₂R₃, or —CO—CH₂CH(OH)—CH₂CH₂CH₂CH₂—N⁺R₁R₂R₃, for instance.

R_{c} can have one or more branches, for example. R_{c} in some aspects can comprise —CHRₛ—(CH₂)ₚ—, where Rₛ is a side chain and p is 0, 1, 2, or 3. Rₛ can be —CH₃, —CH₂CH₃, —CH₂CH₂CH₃, or —CH₂CH₂CH₂CH₃, for example. Rₛ can be —CH₂CH₂CH₂CH₂—N⁺H₃ (i.e., lysine side chain), —CH₂CH₂CH₂CH₂—N⁺ (CH₃)₃, —CH₂CH₂—NH—C(N⁺H₂)—NH₂, —CH₂CH₂CH₂—NH—C(N⁺H₂)—NH₂ (i.e., arginine side chain), or —CH₂-IMD (i.e., histidine side chain, CH₂ bonded to imidazole ring [IMD] carbon-4), for example.

Each of R₁, R₂ and R₃ in some aspects, such as in any of the above structures/formulae, can be as follows: Each of R₁, R₂ and R₃ can be independently selected from —CH₃, —CH₂CH₃, —CH₂CH₂CH₃, or —CH₂CH₂CH₂CH₃, for example (e.g., each of R₁, R₂ and R₃ can be —CH₃). In some aspects, each of R₁, R₂ and R₃ can be independently selected from a mono- or di-hydroxy substituted version of any of these foregoing C1-C4 alkyl groups (e.g., hydroxyethyl such as —CH₂CH₂OH or —CH₂(OH)CH₃). In some aspects, each of R₁, R₂ and R₃ can be independently selected from any of the foregoing C1-C4 alkyl groups and mono- or di-hydroxy substituted versions thereof. In some aspects, R₁ and R₂ can be independently selected from any of the foregoing C1-C4 alkyl groups and mono- or di-hydroxy substituted versions thereof (e.g., R₁ and R₂ can be —CH₃), and R₃ can be as follows: R₃ can be saturated or unsaturated, for example. R₃ can be linear or branched, for example. R₃ can be an alkyl such as —(CH₂)ₙCH₃, where n is 1, 2, 3, 4, 5, 6, 7, 8, 9, 10, 11, 12, 13, 14, 15, 16, 17, 18, 19, 20, 22, 22, or 23, for example (e.g., a C6-C22, C12-14, C10-C16, or C8-C18 alkyl); R₃ can alternatively be an unsaturated version of any of these alkyls, as appropriate. In some aspects, R₃ can be —CH₂CH₂CH₂—NH—CO—CH₂CH₂CH₂—(CH₂)ₙ—CH₂CH₂CH₂CH₃, where n is 0, 2, 4, 6, or 10.

As disclosed above, it should be apparent that a cationic organic group in some aspects can comprise Structure II:

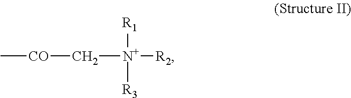

where each of R₁, R₂ and R₃ is independently selected from a group comprising at least one carbon atom (e.g., any of R₁, R₂ and R₃ above). Just to point out for illustration purposes, R_{c} in Structure II is —CH₂—. An example of Structure II herein has R₁, R₂ and R₃ that are each —CH₃. It would be understood that Structure II is a cationic acyl group; further examples of Structure II herein include an acyl (the acyl portion) of any of the following betaine compounds: capramidopropyl betaine, capryl betaine, cetyl amidopropyl betaine, cetyl betaine, cocamidoethyl betaine, cocamidopropyl betaine, decyl amidopropyl betaine, decyl betaine, isostearamidopropyl betaine, lauramidopropyl betaine, lauryl betaine, myristyl amidopropyl betaine, myristyl betaine, oleamidopropyl betaine, oleyl betaine, palmamidopropyl betaine, palmitamidopropyl betaine, stearamidopropyl betaine, stearyl betaine, undecyl betaine, undecylenamidopropyl betaine.

An ester derivative of a polysaccharide/glucan of the present disclosure can be substituted with at least one hydrophobic organic group (hydrophobic acyl group) herein that is ester-linked to the polysaccharide/glucan. A glucan derivative as presently disclosed can be derivatized with one, two, three, or more different types of esterified hydrophobic acyl groups herein, for example. A hydrophobic acyl group can be represented as —CO—R′, wherein R′ is hydrophobic and comprises a chain having at least one carbon atom; the carbonyl (—CO—) of the acyl group is linked to the polysaccharide/glucan monomer (e.g., glucose) via an oxygen atom of the monomer. R′ can be linear, branched, or cyclic, for example. R′ can be saturated or unsaturated, and/or comprise up to 29 carbon atoms, for example.

A hydrophobic acyl group in some aspects can be termed as a “Cₙ acyl group” (or other like terms), where n is an integer of 2 or greater and represents the number of carbon atoms in the acyl group, including the carbonyl carbon atom. A Cₙ acyl group typically is linear, and can be either saturated or unsaturated. The first carbon (carbon-1) of a Cₙ acyl group is its carbonyl carbon. In some aspects, a Cₙ acyl group can be an ethanoyl (C₂), propanoyl (C3), butanoyl (C₄), pentanoyl (C₅), hexanoyl (C₆), heptanoyl (C₇), octanoyl (C₈), nonanoyl (C₉), decanoyl (C₁₀), undecanoyl (C₁₁), dodecanoyl (C₁₂), tridecanoyl (C₁₃), tetradecanoyl (C₁₄), pentadecanoyl (C₁₅), hexadecanoyl (C₁₆), heptadecanoyl (C₁₇), octadecanoyl (C₁₈), nonadecanoyl (C₁₉), eicosanoyl (C₂₀), uneicosanoyl (C₂₁), docosanoyl (C₂₂), tricosanoyl (C₂₃), tetracosanoyl (C₂₄), pentacosanoyl (C₂₅), hexacosanoyl (C₂₆), C₂₇, C₂₈, C₂₉, or C₃₀ acyl group. These particular Cₙ acyl groups are saturated. Common names for some of the above-listed acyl groups are acetyl (ethanoyl group), propionyl (propanoyl group), butyryl (butanoyl group), valeryl (pentanoyl group), caproyl (hexanoyl group); enanthyl (heptanoyl group), caprylyl (octanoyl group), pelargonyl (nonanoyl group), capryl (decanoyl group), lauroyl (dodecanoyl group), myristyl (tetradecanoyl group), palmityl (hexadecanoyl group), stearyl (octadecanoyl group), arachidyl (eicosanoyl group), behenyl (docosanoyl group), lignoceryl (tetracosanoyl group), and cerotyl (hexacosanoyl group). In some aspects, an acyl group can be a C₁₀ to C₁₄ acyl group, meaning that the acyl group can be any one of a C₁₀, C₁₁, C₁₂, C₁₃, or C₁₄ acyl group (this particular Cₙ range nomenclature applies, accordingly, to other Cₙ ranges herein). In some aspects, an acyl group can be a C₂ to C₂₆, C₄ to C₂₀, C₆ to C₁₈, C₈ to C₁₈, C₁₀ to C₁₈, C₁₂ to C₁₈, C₆ to C₁₆, C₈ to C₁₆, C₁₀ to C₁₆, C₁₂ to C₁₆, C₆ to C₁₄, C₈ to C₁₄, C₁₀ to C₁₄, C₁₂ to C₁₄, C₆ to C₁₂, C₈ to C₁₂, or C₁₀ to C₁₂ acyl group.

A hydrophobic acyl group in some aspects can be unsaturated. An unsaturated acyl group can comprise one, two, three, four, five, six, or more double-bonds, for example. An unsaturated acyl group in some aspects can comprise one or more double-bonds spanning carbons (i) 4 and 5), (ii) 5 and 6, (iii) 6 and 7, (iv) 8 and 9, (v) 9 and 10, (vi) 11 and 12, (vii) 12 and 13, (viii) 14 and 15, (ix) 15 and 16, (x) 16 and 17, (xi) 17 and 18, and/or (xii) 18 and 19 of the acyl group, where carbon number is counted starting from the carbonyl carbon (i.e., carbon-1) of the acyl group. Some suitable combinations of double-bonds of an acyl group are as reflected in the below list of unsaturated acyl groups. While a double-bond herein of an acyl group can be in a cis or trans orientation, it typically is in the cis orientation. An unsaturated acyl group can be derived (derivable) from a fatty acid in some aspects. Examples of unsaturated acyl groups herein include (11Z,14Z)-icosadienoyl, (11Z,14Z,17Z)-icosatrienoyl, (4Z)-hexadecenoyl, (4Z,7Z,10Z,13Z,16Z)-docosapentaenoyl, (4Z,7Z,10Z,13Z,16Z,19Z)-docosahexaenoyl, (5Z,8Z,11Z,14Z,17Z)-icosapentaenoyl, (5Z,9Z,12Z)-octadecatrienoyl, (5Z,9Z,12Z,15Z)-octadecatetraenoyl, (6Z,9Z,12Z,15Z)-octadecatetraenoyl, (7Z,10Z)-hexadecadienoyl, (7Z,10Z,13Z)-hexadecatrienoyl, (7Z,10Z,13Z,16Z)-docosatetraenoyl, (7Z,10Z,13Z,16Z,19Z)-docosapentaenoyl, (8E,10E,12Z)-octadecatrienoyl, (8Z,11Z,14Z)-icosatrienoyl, (8Z,11Z,14Z,17Z)-icosatetraenoyl, (9Z)-octadec-9-en-12-ynoyl, (9Z,11E,13E)-octadecatrienoyl, (9Z,11E,13Z)-octadeca-9,11,13-trienoyl, (9Z,12E)-hexadecadienoyl, (9Z,12E)-octadecadienoyl, (9Z,12Z)-octadeca-9, 12-dien-6-ynoyl, (9Z,12Z,15Z)-octadeca-9,12, 15-trien-6-ynoyl, (Z)-tetradec-7-enoyl, cis, cis-tetradeca-5,8-dienoyl, cis-tetradec-5-enoyl, arachidonoyl, docosenoyl, dodecenoyl, eleostearoyl, heptatrienoyl, icosenoyl, linoleoyl, myristoleoyl, octadec-9-ynoyl, octadecenoyl, palmitoleoyl, and oleoyl.

A hydrophobic acyl group in some aspects can comprise an aryl group. An aryl acyl group can comprise a benzoyl group (—CO—C₆H₅), for example, which can also be referred to as a benzoate group. An aryl acyl group in some aspects can comprise a benzoyl group substituted with at least one halogen (“X”; e.g., Cl, F), alkyl, halogenated alkyl, ether, cyano, or aldehyde group, or combinations thereof, such as represented by the following Structures III(a) through III(r):

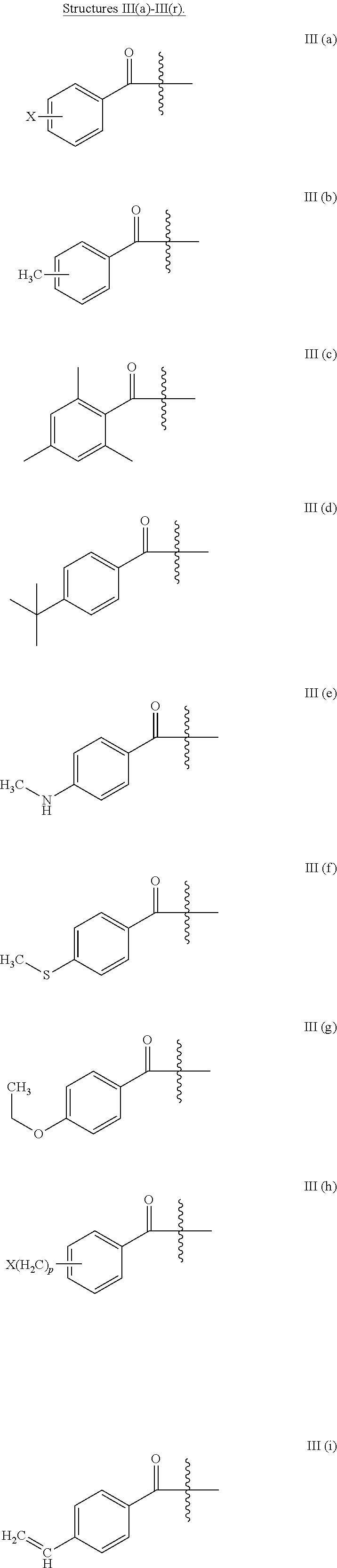

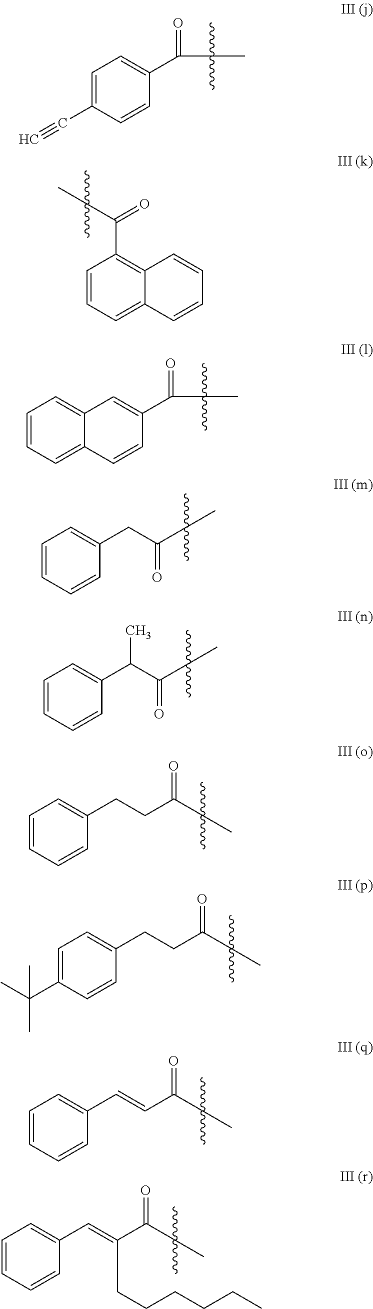

A hydrophobic acyl group in some aspects can comprise a branched group. Examples herein of acyl groups that are branched include 2-methylpropanoyl, 2-methylbutanoyl, 2,2-dimethylpropanoyl, 3-methylbutanoyl, 2-methylpentanoyl, 3-methylpentanoyl, 4-methylpentanoyl, 2,2-dimethylbutanoyl, 2,3-dimethylbutanoyl, 3,3-dimethylbutanoyl, 2-ethylbutanoyl and 2-ethylhexanoyl.

A polysaccharide/glucan ester derivative of the present disclosure can be characterized in some aspects to be a mixed ester by virtue of comprising at least one cationic ester group herein and at least one hydrophobic ester group herein. Merely as examples, a mixed glucan ester can comprise a betaine acyl group herein (e.g., Structure II, where R₁, R₂ and R₅ are each —CH₃) (e.g., DoS of about 0.01-0.12, 0.03-0.1, 0.04-0.09, or 0.05-0.09) and one or both of (i) a C₁₀ to C₁₄ acyl group (e.g., a C₁₂ acyl group such as a lauroyl group) herein and/or (ii) an aryl acyl group (e.g., a benzoyl group) herein as hydrophobic acyl group(s) (e.g., DoS of about 0.2-1.0, 0.3-0.9, 0.4-0.8, or 0.5-0.8); optionally, such a mixed glucan ester can further comprise an acetyl group (e.g., DoS of about 0.02-0.3). In some aspects, such a glucan ester can comprise alpha-1,2- and/or alpha-1,3-branched (e.g., about 15-25% branched) alpha-1,6-glucan (e.g., about 10-70, 20-60, or 30-50 kDa) as its glucan component. While a polysaccharide/glucan ester derivative in some aspects does not comprise any other type of substitution group aside from ester groups, one or more other types of substitution group can be present in other aspects.

Hydrophobic acyl groups of an polysaccharide/glucan ester derivative herein can be as disclosed, for example, in U.S. Patent Appl. Publ. Nos. 2014/0187767, 2018/0155455, or 2020/0308371, or International Patent Appl. Publ. No. WO2021/252575, which are each incorporated herein by reference.

In some alternative aspects, a polysaccharide/glucan as presently disclosed can be derivatized with an anionic organic group and a hydrophobic ester group (i.e., the anionic organic group replaces, or is in addition to, a cationic ester group). A hydrophobic ester group can be as disclosed herein, for example (e.g., an aryl-comprising ester group such as benzoyl). An anionic organic group typically is linked to a polysaccharide/glucan via ether or ester linkage; the anionic organic group can optionally be characterized as an anionic ether group or anionic ester group. Examples of suitable anionic groups herein include carboxyalkyl groups (e.g., carboxymethyl group), which are examples of ether groups, and groups derived from cyclic organic anhydrides (e.g., succinate group), which are examples of ester groups. Merely as examples, a polysaccharide/glucan derivative herein can have (i) carboxymethyl ether and benzoyl ester groups, or (ii) succinate ester and benzoyl ester groups. Suitable anionic groups can be as disclosed in U.S. Pat. Appl. Publ. Nos. 2014/0179913, 2016/0304629, 2020/0002646, 2021/0253977, 2018/0155455, or 2023/0192905, or Int. Pat. Appl. Publ. Nos. WO2021/247810, WO2022/178073, or WO2022/178075, for example, which are incorporated herein by reference. The DoS of a glucan herein with an anionic organic group in the foregoing alternative aspects can be as disclosed herein for a hydrophobic ester group or cationic ester group, for example.

In some alternative aspects, a polysaccharide/glucan as presently disclosed can be derivatized with an anionic organic group and a cationic ester group (i.e., the anionic organic group replaces, or is in addition to, a hydrophobic ester group). A cationic ester group can be as disclosed herein, for example (e.g., a cationic ester group herein comprising Structure I). An anionic organic group typically is linked to a polysaccharide/glucan via ether or ester linkage; the anionic organic group can optionally be characterized as an anionic ether group or anionic ester group. Examples of suitable anionic groups herein include carboxyalkyl groups (e.g., carboxymethyl group), and groups derived from cyclic organic anhydrides (e.g., succinate group). Merely as examples, a polysaccharide/glucan derivative herein can have (i) carboxymethyl ether and Structure I ester groups, or (ii) succinate ester and Structure I ester groups. Suitable anionic groups can be as disclosed in the abovementioned patent application references, for example. The DoS of a glucan herein with an anionic organic group in the foregoing alternative aspects can be as disclosed herein for a hydrophobic ester group or cationic ester group, for example.

Some aspects of the present disclosure concern a method of producing an ester derivative of a glucan herein. Such a method (ester derivatization method/reaction, or esterification method/reaction) can comprise: (a) contacting a glucan with at least two esterification agents, wherein at least one of the esterification agents comprises a cationic organic group (cationic acyl group), wherein at least one of the esterification agents comprises a hydrophobic organic group (hydrophobic acyl group), wherein at least one cationic organic group and at least one hydrophobic organic group are esterified to the glucan, thereby producing an ester derivative of the glucan, wherein the ester derivative of the glucan has a degree of substitution (DoS) up to about 3.0 with the cationic organic group and the hydrophobic organic group, and (b) optionally, isolating the ester derivative of the glucan produced in step (a). Any glucan or other polysaccharide as presently disclosed can be entered into an esterification method, accordingly, to produce any ester derivative herein.

An esterification agent for an ester derivatization method of the present disclosure can be a carboxylic acid comprising any cationic acyl group as disclosed herein, for example. It would be understood that the terminal carbonyl (—CO—) of a cationic acyl group is the carbonyl of the —COOH group of the carboxylic acid comprising the cationic acyl group; here, use of the term “terminal” distinguishes from any internal carbonyl, if present, of an acyl group as presently disclosed. A carboxylic acid can be provided as a salt with an anion such as chloride, fluoride, or bromide, where the anion is in counterbalance to the N⁺ portion(s) of the carboxylic acid.

An esterification agent for an ester derivatization method of the present disclosure can be a carboxylic acid comprising any hydrophobic acyl group as disclosed herein, for example. It would be understood that the terminal carbonyl (—CO—) of a hydrophobic acyl group is the carbonyl of the —COOH group of the carboxylic acid comprising the hydrophobic acyl group.

An esterification agent for an ester derivatization method of the present disclosure can be an acyl halide (acid halide) comprising any acyl group as disclosed herein, for example. The halide of an acyl halide herein can be chloride, fluoride, or bromide, for example. An esterification agent for an ester derivatization method in some aspects can be an acid anhydride comprising any acyl group as disclosed herein, for example. Some illustrative examples of an acid anhydride include aroyl anhydride (aroic anhydride) (e.g., benzoic anhydride [benzoyl anhydride]), acetic anhydride, propionic anhydride and butyric anhydride.

The concentration of an esterification agent (for cationic esterification or hydrophobic esterification) in an esterification reaction herein can be about 10, 25, 50, 75, 100, 125, 150, 175, 200, 10-200, 25-200, 25-100, 10-25, 100-200, or 150-200 g/L, for example.

The step of contacting a glucan with at least one esterification agent is typically performed under substantially anhydrous conditions. A substantially anhydrous esterification reaction herein contains no water or less than about 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 wt % water, for example. A solvent for contacting a glucan with at least one esterification agent can be a non-aqueous solvent, for example, in which the glucan typically can be dissolved. In some aspects, a non-aqueous solvent can be an organic solvent comprising N,N-dimethylacetamide (DMAc) (optionally with about 0.5%-5% LiCl), dimethyl sulfoxide (DMSO), N,N-dimethylformamide (DMF), pyridine, SO₂/diethylamine (DEA)/DMSO, LiCl/1,3-dimethyl-2-imidazolidinone (DMI), DMSO/tetrabutyl-ammonium fluoride trihydrate (TBAF), N-methylpyrrolidone, methylene chloride, and/or N-methylmorpholine-N-oxide (NMMO). A dehydrating agent (e.g., tosyl chloride or dicyandiamide) can optionally be included in a contacting step herein.

A contacting step can be performed in one esterification reaction (“one pot” reaction), for example. In some aspects of a one-pot reaction, at least one cationic esterification agent and at least one hydrophobic esterification agent can be provided in the reaction simultaneously, or these agents can be provided sequentially to the reaction (e.g., one or more hydrophobic esterification agents can be added first, followed by addition of one or more cationic esterification agents, or vice versa). In some aspects, a contacting step can be conducted over at least two separate reactions in which at least one of the reactions uses at least one cationic esterification agent, and at least one of the reactions uses at least one hydrophobic esterification agent. An ester product of the first reaction can optionally be isolated before entering into another reaction. In some aspects, one or more reactions for cationic esterification can be conducted, after which the ester product is entered into one or more reactions for hydrophobic esterification (or vice versa).

The concentration of glucan in an esterification reaction herein can be about, or at least about, 10, 25, 50, 75, 100, 150, 200, 250, 300, 10-300, 10-250, 10-200, 10-50, 25-300, 25-250, 25-200, 25-50, 150-300, 150-250, or 150-200 g/L, for example. The temperature of an esterification reaction herein can be about, or at least about, 50, 60, 70, 80, 90, 100, 110, 120, 130, 140, 150, 50-150, 50-140, 50-130, 60-150, 60-140, 60-130, 70-150, 70-140, 70-130, 60-80, or 110-130° C., for example. In some aspects, an esterification reaction can proceed for about 1, 2, 3, 4, 5, 6, 7, 8, 1-8, 2-8, 1-6, or 2-6 hours. The pH of an esterification reaction can be about 8.5, 9.0, 9.5, 10.0, 10.5, 11.0, 11.5, or 12 in some aspects.

An esterified glucan derivative produced in one or more esterification reactions herein can optionally be isolated. In some aspects, such a product can first be precipitated from the reaction. Precipitation can be performed by adding an excess amount (e.g., at least 2-3 times the volume of the reaction volume) of an alcohol (e.g., 100% or 95% concentration) (e.g., methanol, ethanol, isopropanol) or other solvent (e.g., acetonitrile, ethyl acetate) to the reaction. A precipitated product can then be isolated using a filtration funnel, centrifuge, press filter, or any other method or equipment that allows for removal of liquids from solids. The isolated product can be dried, such as by vacuum drying, air drying, or freeze drying.

In some aspects, an esterified glucan derivative product can be isolated by including a step in which the completed reaction, or a diluted form thereof, is filtered by ultrafiltration (e.g., with a 5 or 10 molecular weight cut-off filter). Optionally, a complete reaction or diluted form thereof can first be regularly filtered (i.e., not ultrafiltration), and then the filtrate can be subjected to ultrafiltration. The concentrated liquid obtained by ultrafiltration can then be dried down to its constituent solids such as by freeze-drying, or the solids can be precipitated from the liquid and then dried (e.g., freeze-drying).

An esterified glucan derivative product herein can optionally be washed, following precipitation or drying, one or more times with a liquid that does not readily dissolve the product. For example, an glucan ester product can be washed with alcohol, acetone, aromatics, or any combination of these, depending on the solubility of the ester product therein (where lack of solubility is desirable for washing). In general, a solvent comprising an organic solvent (e.g. 95-100%) such as alcohol is preferred for washing a glucan ester derivative product.

Any of the above esterification reactions can be repeated using a glucan ester derivative product herein as the starting material for further modification. Such further modification can be with the same esterification agent used in the first reaction, or with a different esterification agent.

A composition as presently disclosed comprising at least one glucan ester derivative herein can be an aqueous composition (e.g., a solution, or a dispersion such as colloidal dispersion) or a dry composition, for example. In some aspects, a composition herein can comprise about, at least about, or less than about, 0.01, 0.05, 0.1, 0.2, 0.25, 0.3, 0.4, 0.5, 0.6, 0.7, 0.75, 0.8, 0.9, 1.0, 1.2, 1.25, 1.4, 1.5, 1.6, 1.75, 1.8, 2.0, 2.25, 2.5, 3.0, 3.5, 4.0, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 99.5 wt % or w/v % of a glucan ester derivative. A composition can comprise a range between any two of these wt % or w/v % values (e.g., 5-50, 5-45, 5-40, 5-35, 5-30, 5-25, 5-20, 5-15, 5-10, 1-20, 1-15, 1-10, 1-7.5, 1-5, 2-20, 2-15, 2-10, 2-7.5, 2-5, 3-20, 3-15, 3-10, 3-7.5, or 3-5 wt % or w/V %), for example. The liquid component of an aqueous composition can be an aqueous fluid such as water or aqueous solution, for instance. The solvent of an aqueous solution typically is water, or can comprise about, or at least about, 10, 20, 30, 40, 50, 60, 70, 80, 90, 95, 98, or 99 wt % water, for example, or is as disclosed below. In some aspects, a composition herein can comprise, or be in the form of, a solution, dispersion (e.g., emulsion), mixture, wet cake or wet powder, dry powder, extrusion, composite, film/coating, fiber, or fibrid.

A solvent of a composition herein can, in some aspects, comprise water and at least about 40% (v/v or w/w) of one or more polar organic solvents, for example. In some aspects, a solvent comprises about, or at least about, 40, 41, 42, 43, 44, 45, 46, 47, 48, 49, 50, 51, 52, 53, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 40-90, 40-80, 40-70, 40-60, 50-90, 50-80, 50-70, 50-60, 60-90, 60-80, 60-70, 70-90, 70-80, 40-70, 40-60, 75-85, or 85-95 v/v % or w/w % of one or more polar organic solvents. The balance of a solvent typically is water only (e.g., a solvent with about 75 v/v % polar organic solvent has about 25 v/v % water), but can optionally comprise (e.g., less than 2, 1, 0.5, or 0.25 v/v %) one or more other liquids aside from a polar organic solvent. A solvent herein can optionally be characterized as an aqueous solvent given its having water. While a solvent herein typically comprises one type of polar organic solvent, two, three, or more polar organic solvents can optionally be included; in such aspects, the polar organic solvent concentration typically is that of the combination of the polar organic solvents.

A polar organic solvent in some aspects can be protic. Examples of protic polar organic solvents herein include an alcohol (e.g., methanol, ethanol, isopropanol, 1-propanol, tert-butyl alcohol, n-butanol, iso-butanol), methyl formamide and formamide. Additional examples of protic polar organic solvents herein include n-butanol, ethylene glycol, 2-methoxyethanol, 1-methoxy-2-propanol, glycerol, 1,2-propanediol, and 1,3-propanetriol.

A polar organic solvent in some aspects can be aprotic. Examples of aprotic polar organic solvents herein include acetonitrile, dimethyl sulfoxide, acetone, N,N-dimethylformamide, N,N-dimethylacetamide, tetrahydrofuran, propylene carbonate, and sulfolane. Additional examples of aprotic polar organic solvents herein include hexamethylphosphoramide, dimethylimidazolidinone (1,3-dimethyl-2-imidazolidinone), dioxane, nitromethane, and butanone. In general, ester, ketone and aldehyde solvents having no acidic hydrogen atom are other examples of aprotic polar organic solvents herein.

An aqueous composition herein can have a viscosity of about, at least about, or less than about, 1, 5, 10, 100, 200, 300, 400, 500, 600, 700, 1000, 2000, 3000, 4000, 5000, 6000, 7000, 8000, 9000, 10000, 15000, 1-300, 10-300, 25-300, 50-300, 1-250, 10-250, 25-250, 50-250, 1-200, 10-200, 25-200, 50-200, 1-150, 10-150, 25-150, 50-150, 1-100, 10-100, 25-100, or 50-100 centipoise (cps), for example. Viscosity can be as measured with an aqueous composition herein at any temperature between about 3° C. to about 80° C., for example (e.g., 4-30° C., 15-30° C., 15-25° C.). Viscosity typically is as measured at atmospheric pressure (about 760 torr) or a pressure that is +10% thereof. Viscosity can be measured using a viscometer or rheometer, for example, and can optionally be as measured at a shear rate (rotational shear rate) of about 0.1, 0.5, 1.0, 5, 10, 50, 100, 500, 1000, 0.1-500, 0.1-100, 1.0-500, 1.0-1000, or 1.0-100 s⁻¹ (1/s), or about 5, 10, 20, 25, 50, 100, 200, or 250 rpm (revolutions per minute), for example.

A composition as presently disclosed can have a turbidity of about, or less than about, 1500, 1400, 1300, 1200, 1100, 1000, 900, 800, 700, 600, 500, 400, 300, 280, 260, 240, 220, 200, 190, 180, 170, 160, 150, 140, 130, 120, 110, 100, 90, 80, 70, 60, 50, 45, 40, 35, 30, 25, 20, 18, 16, 14, 12, 10, 9, 8, 7, 6, 5, 4, 3, 2, 1, 1-250, 1-200, 1-150, 1-100, 1-50, 1-20, 1-15, 1-10, 1-5, 2-250, 2-200, 2-150, 2-100, 2-50, 2-20, 2-15, 2-10, 2-5, 10-250, 10-200, 10-150, 10-100, 10-50, or 10-20 NTU (nephelometric turbidity units), for example. Any of these NTU values can optionally be with respect to an alpha-glucan ester derivative and solvent ingredients portion of a composition herein. In some aspects, any of these NTU levels is contemplated to be (to persist) for a time (typically beginning from initial preparation) of about, at least about, or up to about, 0.5, 1, 2, 4, 6, 8, 10, 20, 30, 60, 90, 120, 150, 180, 210, 240, 270, 300, 330, or 360 days, or 1, 2, or 3 years. Any suitable method can be used to measure turbidity, such as the methodology disclosed in Progress in Filtration and Separation (Edition: 1, Chapter 16. Turbidity: Measurement of Filtrate and Supernatant Quality?, Publisher: Academic Press, Editors: E.S. Tarleton, July 2015), which is incorporated herein by reference, or as described in the below Examples.

The aqueous solution component of an aqueous composition in some aspects has no (detectable) dissolved sugars, or about 0.1-1.5, 0.1-1.25, 0.1-1.0, 0.1-0.75, 0.1-0.5, 0.2-0.6, 0.3-0.5, 0.2, 0.3, 0.4, 0.5, or 0.6 wt % dissolved sugars. Such dissolved sugars can include sucrose, fructose, leucrose, and/or soluble gluco-oligosaccharides, for example. The aqueous solution component of an aqueous composition in some aspects can have one or more salts/buffers (e.g., Na⁺, Cl⁻, NaCl, phosphate, tris, citrate) (e.g., ≤0.1, 0.5, 1.0, 2.0, or 3.0 wt %), and/or a pH of about 4.0, 4.5, 5.0, 5.5, 6.0, 6.5, 7.0, 7.5, 8.0, 8.5, 9.0, 9.5, 10.0, 10.5, 4.0-10.0, 4.0-9.0, 4.0-8.0, 5.0-10.0, 5.0-9.0, 5.0-8.0, 6.0-10.0, 6.0-9.0, or 6.0-8.0, for example. In some aspects, such as with an ester herein of an insoluble glucan (e.g., alpha-1,3-glucan of DP>8 or >9), the glucan ester is insoluble in aqueous conditions with a pH of at least about 10, 10.5, or 11 (e.g., at a concentration of at least about 0.5 or 1.0 wt %).

In some aspects, with an aqueous composition that is an aqueous dispersion (e.g., emulsion) of particles of glucan ester of the present disclosure, the particles are dispersed through about, or at least about, 30%, 40%, 50%, 60%, 70%, 80%, 85%, 90%, 95%, 98%, 99%, or 100% of the volume of the dispersion. In some aspects, such a level of dispersion (e.g., emulsion) is contemplated to be for a time (typically beginning from initial preparation of the dispersion) of about, at least about, or up to about, 0.5, 1, 2, 4, 6, 8, 10, 20, 30, 60, 90, 120, 150, 180, 210, 240, 270, 300, 330, or 360 days, or 1, 2, or 3 years.

The temperature of a composition herein can be about, at least about, or up to about, 0, 5, 10, 15, 20, 25, 30, 35, 40, 45, 50, 55, 60, 65, 70, 75, 80, 85, 90, 95, 100, 105, 110, 115, 120, 125, 130, 5-50, 20-25, 20-30, 20-40, 30-40, 40-130, 40-125, 40-120, 70-130, 70-125, 70-120, 80-130, 80-125, 80-120, 60-100, 60-90, 70-100, 70-90, 75-100, 75-90, or 75-85° C., for example.

A composition herein can, in some aspects, be non-aqueous (e.g., a dry composition). Examples of such embodiments include powders, granules, microcapsules, flakes, or any other form of particulate matter. Other examples include larger compositions such as pellets, bars, kernels, beads, tablets, sticks, or other agglomerates, or ointment or lotion (or any other form herein of a non-aqueous or dry composition). A non-aqueous or dry composition typically has about, or no more than about, 12, 10, 8, 6, 5, 4, 3, 2, 1.5, 1.0, 0.5, 0.25, 0.10, 0.05, or 0.01 wt % water comprised therein. In some aspects (e.g., those directed to a laundry or dish washing detergent), a dry composition herein can be provided in a sachet or pouch.

A composition herein comprising a glucan ester derivative can, in some aspects, be a detergent composition. Examples of such compositions are disclosed herein as detergents for dishwashing and detergents for fabric care.

A composition herein can, in some aspects, comprise one or more salts such as a sodium salt (e.g., NaCl, Na₂SO₄). Other non-limiting examples of salts include those having (i) an aluminum, ammonium, barium, calcium, chromium (II or III), copper (I or II), iron (II or III), hydrogen, lead (II), lithium, magnesium, manganese (II or III), mercury (I or II), potassium, silver, sodium strontium, tin (II or IV), or zinc cation, and (ii) an acetate, borate, bromate, bromide, carbonate, chlorate, chloride, chlorite, chromate, cyanamide, cyanide, dichromate, dihydrogen phosphate, ferricyanide, ferrocyanide, fluoride, hydrogen carbonate, hydrogen phosphate, hydrogen sulfate, hydrogen sulfide, hydrogen sulfite, hydride, hydroxide, hypochlorite, iodate, iodide, nitrate, nitride, nitrite, oxalate, oxide, perchlorate, permanganate, peroxide, phosphate, phosphide, phosphite, silicate, stannate, stannite, sulfate, sulfide, sulfite, tartrate, or thiocyanate anion. Thus, any salt having a cation from (i) above and an anion from (ii) above can be in a composition, for example. A salt can be present in an aqueous composition herein at a wt % of about, or at least about, 0.01, 0.025, 0.05, 0.075, 0.1, 0.25, 0.5, 0.75, 1.0, 1.25, 1.5, 1.75, 2.0, 2.5, 3.0, 3.5, 0.01-3.5, 0.5-3.5, 0.5-2.5, or 0.5-1.5 wt % (such wt % values typically refer to the total concentration of one or more salts), for example.

A composition herein can optionally contain one or more enzymes (active enzymes). Examples of suitable enzymes include proteases, cellulases, hemicellulases, peroxidases, lipolytic enzymes (e.g., metallolipolytic enzymes), xylanases, lipases, phospholipases, esterases (e.g., arylesterase, polyesterase), perhydrolases, cutinases, pectinases, pectate lyases, mannanases, keratinases, reductases, oxidases (e.g., choline oxidase), phenoloxidases, lipoxygenases, ligninases, pullulanases, tannases, pentosanases, malanases, beta-glucanases, arabinosidases, hyaluronidases, chondroitinases, laccases, metalloproteinases, amadoriases, glucoamylases, arabinofuranosidases, phytases, isomerases, transferases, nucleases, and amylases. If an enzyme(s) is included, it may be comprised in a composition herein at about 0.0001-0.1 wt % (e.g., 0.01-0.03 wt %) active enzyme (e.g., calculated as pure enzyme protein), for example. In fabric care or automatic dishwashing applications, an enzyme herein (e.g., any of the above such as cellulase, protease, amylase, and/or lipase) can be present in an aqueous composition in which a fabric or dish is treated (e.g., wash liquor, grey water) at a concentration that is minimally about 0.01-0.1 ppm total enzyme protein, or about 0.1-10 ppb total enzyme protein (e.g., less than 1 ppm), to maximally about 100, 200, 500, 1000, 2000, 3000, 4000, or 5000 ppm total enzyme protein, for example.

A glucan ester derivative and/or a composition comprising such a derivative is biodegradable in some aspects. Such biodegradability can be, for example, as determined by the Carbon Dioxide Evolution Test Method (OECD Guideline 301B, incorporated herein by reference), to be about, at least about, or at most about, 5%, 10%, 15%, 20%, 25%, 30%, 35%, 40%, 45%, 50%, 55%, 60%, 65%, 70%, 75%, 80%, 85%, 90%, 5-60%, 5-80%, 5-90%, 40-70%, 50-70%, 60-70%, 40-75%, 50-75%, 60-75%, 70-75%, 40-80%, 50-80%, 60-80%, 70-80%, 40-85%, 50-85%, 60-85%, 70-85%, 40-90%, 50-90%, 60-90%, or 70-90%, or any value between 5% and 90%, after 15, 30, 45, 60, 75, or 90 days of testing. It is contemplated that such biodegradability can be about, at least about, or at most about, 10%, 25%, 50%, 75%, 100%, 150%, 200%, 250%, 500%, 750%, or 1000% higher than the biodegradability of an incumbent material.

A composition can comprise one, two, three, four or more different glucan ester derivatives herein and, optionally, at least one non-derivatized glucan (e.g., as disclosed herein). For example, a composition can comprise at least one type of glucan ester derivative and at least one type of glucan; in some aspects, the latter can be (or can be capable of being) a precursor compound of the former. In some aspects, a non-derivatized alpha-glucan (e.g., precursor compound) is not present.

A composition as presently disclosed comprising at least one glucan ester derivative can be in the form of a household care product, personal care product, industrial product, ingestible product (e.g., food product), medical product, or pharmaceutical product, for example, such as described in any of U.S. Patent Appl. Publ. Nos. 2018/0022834, 2018/0237816, 2018/0230241, 20180079832, 2016/0311935, 2016/0304629, 2015/0232785, 2015/0368594, 2015/0368595, 2016/0122445, 2019/0202942, or 2019/0309096, or International Patent Appl. Publ. No. WO2016/133734, which are all incorporated herein by reference. In some aspects, a composition can comprise at least one component/ingredient of a household care product, personal care product, industrial product, pharmaceutical product, or ingestible product (e.g., food product) as disclosed in any of the foregoing publications and/or as presently disclosed.

A composition in some aspects is believed to be useful for providing one or more of the following physical properties to a personal care product, pharmaceutical product, household product, industrial product, or ingestible product (e.g., food product): thickening, freeze/thaw stability, lubricity, moisture retention and release, texture, consistency, shape retention, emulsification, binding, suspension, dispersion, gelation, or reduced mineral hardness, for example.

Personal care products herein are not particularly limited and include, for example, skin care compositions, cosmetic compositions, antifungal compositions, and antibacterial compositions. Personal care products herein may be in the form of, for example, lotions, creams, pastes, balms, ointments, pomades, gels, liquids, combinations of these and the like. The personal care products disclosed herein can include at least one active ingredient, if desired. An active ingredient is generally recognized as an ingredient that causes an intended pharmacological effect.

In some aspects, a skin care product can be applied to skin for addressing skin damage related to a lack of moisture. A skin care product may also be used to address the visual appearance of skin (e.g., reduce the appearance of flaky, cracked, and/or red skin) and/or the tactile feel of the skin (e.g., reduce roughness and/or dryness of the skin while improved the softness and subtleness of the skin). A skin care product typically may include at least one active ingredient for the treatment or prevention of skin ailments, providing a cosmetic effect, or for providing a moisturizing benefit to skin, such as zinc oxide, petrolatum, white petrolatum, mineral oil, cod liver oil, lanolin, dimethicone, hard fat, vitamin A, allantoin, calamine, kaolin, glycerin, or colloidal oatmeal, and combinations of these. A skin care product may include one or more natural moisturizing factors such as ceramides, hyaluronic acid, glycerin, squalane, amino acids, cholesterol, fatty acids, triglycerides, phospholipids, glycosphingolipids, urea, linoleic acid, glycosaminoglycans, mucopolysaccharide, sodium lactate, or sodium pyrrolidone carboxylate, for example. Other ingredients that may be included in a skin care product include, without limitation, glycerides, apricot kernel oil, canola oil, squalane, squalene, coconut oil, corn oil, jojoba oil, jojoba wax, lecithin, olive oil, safflower oil, sesame oil, shea butter, soybean oil, sweet almond oil, sunflower oil, tea tree oil, shea butter, palm oil, cholesterol, cholesterol esters, wax esters, fatty acids, and orange oil. A skin care product can be an ointment, lotion, or sanitizer (e.g., hand sanitizer) in some aspects.

A personal care product herein can also be in the form of makeup, lipstick, mascara, rouge, foundation, blush, eyeliner, lip liner, lip gloss, other cosmetics, sunscreen, sun block, nail polish, nail conditioner, temporary tattoo ink, bath gel, shower gel, body wash, face wash, lip balm, skin conditioner, cold cream, moisturizer, body spray, soap, body scrub, exfoliant, astringent, scruffing lotion, depilatory, permanent waving solution, antidandruff formulation, antiperspirant composition, deodorant, shaving product, pre-shaving product, after-shaving product, cleanser, skin gel, rinse, dentifrice composition, toothpaste, or mouthwash, for example. An example of a personal care product (e.g., a cleanser, soap, scrub, cosmetic) comprises a carrier or exfoliation agent (e.g., jojoba beads [jojoba ester beads]) (e.g., about 1-10, 3-7, 4-6, or 5 wt %); such an agent may optionally be dispersed within the product.

A personal care product in some aspects can be a hair care product. Examples of hair care products herein include shampoo, hair conditioner (leave-in or rinse-out), cream rinse, hair dye, hair coloring product, hair shine product, hair serum, hair anti-frizz product, hair split-end repair product, mousse (e.g., hair styling mousse), hair spray (e.g., hair styling spray), and styling gel (e.g., hair styling gel). A hair care product can be in the form of a liquid, paste, gel, solid, or powder in some embodiments. A hair care product as presently disclosed typically comprises one or more of the following ingredients, which are generally used to formulate hair care products: anionic surfactants such as polyoxyethylenelauryl ether sodium sulfate; cationic surfactants such as stearyltrimethylammonium chloride and/or distearyltrimethylammonium chloride; nonionic surfactants such as glyceryl monostearate, sorbitan monopalmitate and/or polyoxyethylenecetyl ether; wetting agents such as propylene glycol, 1,3-butylene glycol, glycerin, sorbitol, pyroglutamic acid salts, amino acids and/or trimethylglycine; hydrocarbons such as liquid paraffins, petrolatum, solid paraffins, squalane and/or olefin oligomers; higher alcohols such as stearyl alcohol and/or cetyl alcohol; superfatting agents; antidandruff agents; disinfectants; anti-inflammatory agents; crude drugs; water-soluble polymers such as methyl cellulose, hydroxycellulose and/or partially deacetylated chitin; antiseptics such as paraben; ultra-violet light absorbers; pearling agents; pH adjustors; perfumes; and pigments.

A composition in some aspects can be a hair care composition such as a hair styling or hair setting composition (e.g., hair spray, hair gel or lotion, hair mousse/foam) (e.g., aerosol hair spray, non-aerosol pump-spray, spritze, foam, crème, paste, non-runny gel, mousse, pomade, lacquer, hair wax). A hair styling/setting composition/formulation that can be adapted to include at least one glucan ester derivative herein can be as disclosed in, for example, US20090074697, WO1999048462, US20130068849, JPH0454116A, U.S. Pat. No. 5,304,368, AU667246B2, U.S. Pat. Nos. 5,413,775, 5,441,728, 5,939,058, JP2001302458A, U.S. Pat. No. 6,346,234, US20020085988, U.S. Pat. No. 7,169,380, US20090060858, US20090326151, US20160008257, WO2020164769, or US20110217256, all of which are incorporated herein by reference. A hair care composition such as a hair styling/setting composition can comprise one or more ingredients/additives as disclosed in any of the foregoing references, and/or one or more of a fragrance/perfume, aroma therapy essence, herb, infusion, antimicrobial, stimulant (e.g., caffeine), essential oil, hair coloring, dying or tinting agent, anti-gray agent, anti-foam agent, sunscreen/UV-blocker (e.g., benzophenone-4), vitamin, antioxidant, surfactant or other wetting agent, mica, silica, metal flakes or other glitter-effect material, conditioning agent (e.g., a volatile or non-volatile silicone fluid), anti-static agent, opacifier, detackifying agent, penetrant, preservative (e.g., phenoxyethanol, ethylhexylglycerin, benzoate, diazolidinyl urea, iodopropynyl butylcarbamate), emollient (e.g., panthenol, isopropyl myristate), rheology-modifying or thickening polymer (e.g., acrylates/methacrylamide copolymer, polyacrylic acid [e.g., CARBOMER]), emulsified oil phase, petrolatum, fatty alcohols, diols and polyols, emulsifier (e.g., PEG-40 hydrogenated castor oil, Oleth-20), humectant (e.g., glycerin, caprylyl glycol), silicone derivative, protein, amino acid (e.g., isoleucine), conditioner, chelant (e.g., EDTA), solvent (e.g., see below), monosaccharide (e.g., dextrose), disaccharide, oligosaccharide, pH-stabilizing compound (e.g., aminomethyl propanol), film former (e.g., acrylates/hydroxyester acrylate copolymer, polyvinylpyrrolidone/vinyl acetate copolymer, triethyl acetate), aerosol propellant (e.g., C₃-C₅ alkane such as propane, isobutane, or n-butane, monoalkyl ether, dialkyl ether such as di (C₁-C₄ alkyl) ether [e.g., dimethyl ether]), and/or any other suitable material herein. A glucan ester derivative as used in a hair styling/setting composition herein can function as a hair fixing/styling agent (typically non-permanent hair fixing, but durable), for example, and optionally is the only hair fixing agent in the composition. Optional additional hair fixing/styling agents herein include PVP (polyvinylpyrrolidone), octylacrylamide/acrylates/butylaminoethyl methacrylate copolymer, vinyl caprolactam/PVP/dimethylaminoethyl methacrylate copolymer, AMPHOMER, or any film former such as listed above.

The total content of one or more glucan ester derivatives in a hair care composition such as a hair styling/setting composition herein can be about, at least about, or less than about, 0.5, 1, 2, 3, 4, 5, 6, 7, 8, 9, 10, 11, 12, 13, 14, 15, 0.5-15, 0.5-10, 0.5-5, 0.5-2, 1-15, 1-10, 1-5, 1-2, 2.5-7.5, 3-7, or 4-6 wt %, for example. A hair styling/setting composition can comprise a solvent comprising water and optionally a water-miscible (typically polar) organic compound (e.g., liquid or gas) such as an alcohol (e.g., ethanol, propanol, isopropanol, n-butanol, iso-butanol, tert-butanol), an alkylene glycol alkyl ether, and/or a monoalkyl or dialkyl ether (e.g., dimethyl ether), for example. If an organic compound is included, it can constitute about 10%, 20%, 30%, 40%, 50%, or 60% by weight or volume of the solvent (balance is water), for example. The amount of solvent in a hair styling/setting composition herein can be about 50-90, 60-90, 70-90, 80-90, 50-95, 60-95, 70-95, 80-95, or 90-95 wt %, for example.

An example of a hair styling gel formulation herein can comprise about 90-95 wt % (e.g., ~92 wt %) solvent (e.g., any herein), 0.3-1.0 wt % (e.g., ~0.5 wt %) thickener (e.g., polyacrylic acid), 0.1-0.3 wt % (e.g., ~0.2 wt %) chelant (e.g., EDTA) (optional), 0.2-1.0 wt % (e.g., ~0.5 wt %) humectant (e.g., glycerin), 0.01-0.05 wt % (e.g., ~0.02 wt %) UV-blocker (e.g., benzophenone-4) (optional), 0.05-0.3 wt % (e.g., ~0.1 wt %) preservative (e.g., diazolidinyl urea) (optional), 0.5-1.2 wt % (e.g., ~0.8 wt %) emulsifier (e.g., Oleth-20), 0.1-0.3 wt % (e.g., ~0.2 wt %) fragrance/perfume (optional), 0.2-1.0 wt % (e.g., ~0.5 wt %) pH-stabilizing compound (e.g., aminomethyl propanol), and 3-7 wt % (e.g., ~5 wt %) glucan ester derivative herein (e.g., as a hair fixing/styling agent).

An example of a hair styling spray formulation herein can comprise about 0.2-1.0 wt % (e.g., ~0.5 wt %) pH-stabilizing compound (e.g., aminomethyl propanol), 0.1-0.3 wt % (e.g., ~0.2 wt %) fragrance/perfume (optional), 0.05-0.12 wt % (e.g., ~0.08 wt %) surfactant (e.g., ethoxylated dimethicone polyol), 0.05-0.12 wt % (e.g., ~0.08 wt %) conditioner (e.g., cyclomethicone) (optional), 0.05-0.3 wt % (e.g., ~0.2 wt %) preservative (e.g., sodium benzoate) (optional), 15-20 wt % (e.g., ~17 wt %) water, 30-40 wt % (e.g., ~65 wt %) alcohol (e.g., ethanol), 40-60 wt % (e.g., ~45 wt %) propellant (e.g., dimethyl ether, or a ~2:1 mix of dimethyl ether to C₃-C₅ alkane [e.g., mix of propane and isobutane]), and 2-4 wt % (e.g., ~2.75 wt %) glucan ester derivative herein (e.g., as a hair fixing/styling agent).

Some aspects of the present disclosure regard hair that has been treated with a hair care composition herein (e.g., hair styling/setting composition, shampoo, or conditioner). For example, hair can comprise a glucan ester derivative on its surface, such as in a film/coating of the hair, and/or adsorbed or otherwise deposited on the hair surface; optionally, one or more other ingredients of a hair care composition herein can also be present. Typically, hair as presently disclosed, such as hair with a coating comprising an alpha-glucan ester, does not exhibit flaking to the naked eye (i.e., little or no noticeable flaking).

Various examples of personal care formulations comprising at least one glucan ester derivative as presently disclosed are disclosed below (1-3).

(1) A hair conditioner composition comprising: cetyl alcohol (1-3%), isopropyl myristate (1-3%), hydroxyethyl cellulose (Natrosol® 250 HHR, 0.1-1%), glucan ester derivative (0.1-2%), potassium salt (0.1-0.5%), Germaben® II preservative (0.5%, available from International Specialty Products), and the balance being water.

(2) A hair shampoo composition comprising: 5-20% sodium laureth sulfate (SLES), 1-2 wt % cocamidopropyl betaine, 1-2 wt % sodium chloride, 0.1-2% glucan ester derivative, preservative (0.1-0.5%), and the balance being water.

(3) A skin lotion composition comprising: 1-5% glycerin, 1-5% glycol stearate, 1-5% stearic acid, 1-5% mineral oil, 0.5-1% acetylated lanolin (Lipolan® 98), 0.1-0.5 cetyl alcohol, 0.2-1% triethanolamine, 0.1-1 wt % Germaben® II preservative, 0.5-2 wt % glucan ester derivative, and the balance being water.

A pharmaceutical product herein can be in the form of an emulsion, liquid, elixir, gel, suspension, solution, cream, or ointment, for example. Also, a pharmaceutical product herein can be in the form of any of the personal care products disclosed herein, such as an antibacterial or antifungal composition. A pharmaceutical product can further comprise one or more pharmaceutically acceptable carriers, diluents, and/or pharmaceutically acceptable salts. A composition herein can also be used, for example, in capsules, tablets, tablet coatings, and as excipients for medicaments and drugs.

A household and/or industrial product herein can be in the form of drywall tape-joint compounds; mortars; grouts; cement plasters; spray plasters; cement stucco; adhesives; pastes; wall/ceiling texturizers; binders and processing aids for tape casting, extrusion forming, injection molding and ceramics; spray adherents and suspending/dispersing aids for pesticides, herbicides, and fertilizers; fabric care products such as fabric softeners and laundry detergents; hard surface cleaners; air fresheners; polymer emulsions; latex; gels such as water-based gels; surfactant solutions; paints such as water-based paints; protective coatings; adhesives; sealants and caulks; inks such as water-based ink; metal-working fluids; films or coatings; or emulsion-based metal cleaning fluids used in electroplating, phosphatizing, galvanizing and/or general metal cleaning operations, for example. In some aspects, a composition herein is comprised in a fluid as a viscosity modifier and/or friction reducer; such uses include downhole operations/fluids (e.g., in hydraulic fracturing and enhanced oil recovery), for example.

Some aspects herein regard (i) salt water such as seawater, or (ii) an aqueous solution having about 2.0, 2.25, 2.5, 2.75, 3.0, 3.25. 3.5, 3.75, 4.0, 2.5-4.0, 2.75-4.0, 3.0-4.0, 2.5-3.5, 2.75-3.5, 3.0-3.5, 3.0-4.0, or 3.0-3.5 wt % of one or a combination of salts (e.g., including at least NaCl), having at least one aqueous-soluble glucan ester derivative as presently disclosed. The concentration of a glucan ester derivative in such water of (i) or (ii) can be about, at least about, or below about, 0.1, 0.2, 0.3, 0.4, 0.5, 0.6, 0.7, 0.8, 0.9, 1.0, 0.1-0.6, 0.1-0.5, 0.1-0.4, 0.1-0.3, or 0.1-0.2 wt %, for example. Despite the relatively high salt concentration in such aqueous compositions, it is contemplated that a glucan ester derivative in some aspects can remain completely or mostly in solution and provide viscosity. Such a solution of (i) or (ii) as viscosity-modified by a glucan ester derivative herein can be as it is used within a system that utilizes such a solution (e.g., any herein, such as a downhole operation).

Examples of ingestible products herein include a food, beverage, animal feed, an animal health and/or nutrition product, and/or pharmaceutical product. The intended use of a composition as presently disclosed in an ingestible product can be to provide texture, add volume, and/or thicken, for example.

Further examples of using a composition of the present disclosure for ingestible products include use as: a bulking, binding and/or coating ingredient; a carrier for coloring agents, flavors/fragrances, and/or high intensity sweeteners; a spray drying adjunct; a bulking, bodying, dispersing and/or emulsification agent; and an ingredient for promoting moisture retention (humectant). Illustrative examples of products that can be prepared having a composition herein include food products, beverage products, pharmaceutical products, nutritional products, and sports products. Examples of beverage products herein include concentrated beverage mixes, carbonated beverages, non-carbonated beverages, fruit-flavored beverages, fruit juices, teas, coffee, milk nectars, powdered drinks, liquid concentrates, milk drinks, ready-to-drink (RTD) products, smoothies, alcoholic beverages, flavored waters and combinations thereof. Examples of food products herein include baked goods (e.g., breads), confectioneries, frozen dairy products, meats, artificial/synthetic/cultured meat, cereal products (e.g., breakfast cereals), dairy products (e.g., yogurt), condiments (e.g., mustard, ketchup, mayonnaise), snack bars, soups, dressings, mixes, prepared foods, baby foods, diet preparations, peanut butter, syrups, sweeteners, food coatings, pet food, animal feed, animal health and nutrition products, dried fruit, sauces, gravies, jams/jellies, dessert products, spreads, batters, breadings, spice mixes, frostings and the like. In some aspects, a composition herein can provide or enhance the foaming of beverages such as dairy beverages, non-dairy alternative beverages (e.g., “vegan” milk such as soy milk, almond milk, or coconut milk), dairy creamers, and/or non-dairy creamers (e.g., for a hot beverage such as coffee [e.g., cappuccino], tea [e.g., chai tea]).

A composition herein comprising a glucan ester derivative can be comprised in a personal care product, pharmaceutical product, household product, industrial product, or ingestible product (e.g., food product) in an amount that provides a desired degree of thickening and/or dispersion, for example. Examples of a concentration or amount of a disclosed composition in a product are any of the weight percentages provided herein.

In some aspects, a composition comprising at least one glucan ester derivative herein can be in the form of, or comprise, a fabric care composition. A fabric care composition can be used for hand wash, machine wash and/or other purposes such as soaking and/or pretreatment of fabrics, for example. A fabric care composition may take the form of, for example, a laundry detergent; fabric conditioner; any wash-, rinse-, or dryer-added product; unit dose or spray. Fabric care compositions in a liquid form may be in the form of an aqueous composition. In other embodiments, a fabric care composition can be in a dry form such as a granular detergent or dryer-added fabric softener sheet. Other non-limiting examples of fabric care compositions can include: granular or powder-form all-purpose or heavy-duty washing agents; liquid, gel or paste-form all-purpose or heavy-duty washing agents; liquid or dry fine-fabric (e.g. delicates) detergents; cleaning auxiliaries such as bleach additives, “stain-stick”, or pre-treatments; substrate-laden products such as dry and wetted wipes, pads, or sponges; sprays and mists; water-soluble unit dose articles. As further examples, a composition herein can be in the form of a liquid, a gel, a powder, a hydrocolloid, an aqueous solution, a granule, a tablet, a capsule, a bead or pastille, a single compartment sachet, a multi-compartment sachet, a single compartment pouch, or a multi-compartment pouch.

A detergent composition herein may be in any useful form, e.g., as powders, granules, pastes, bars, unit dose, or liquid. A liquid detergent may be aqueous, typically containing up to about 70 wt % of water and 0 wt % to about 30 wt % of organic solvent. It may also be in the form of a compact gel type containing only about 30 wt % water.

A detergent composition (e.g., of a fabric care product or any other product herein) typically comprises one or more surfactants, wherein the surfactant is selected from nonionic surfactants, anionic surfactants, cationic surfactants, ampholytic surfactants, zwitterionic surfactants, semi-polar nonionic surfactants and mixtures thereof. In some embodiments, the surfactant is present at a level of from about 0.1% to about 60%, while in alternative embodiments the level is from about 1% to about 50%, while in still further embodiments the level is from about 5% to about 40%, by weight of the detergent composition. A detergent will usually contain 0 wt % to about 50 wt % of an anionic surfactant such as linear alkylbenzenesulfonate (LAS), alpha-olefinsulfonate (AOS), alkyl sulfate (fatty alcohol sulfate) (AS), alcohol ethoxysulfate (AEOS or AES), secondary alkanesulfonates (SAS), alpha-sulfo fatty acid methyl esters, alkyl- or alkenylsuccinic acid, or soap. In addition, a detergent composition may optionally contain 0 wt % to about 40 wt % of a nonionic surfactant such as alcohol ethoxylate (AEO or AE), carboxylated alcohol ethoxylates, nonylphenol ethoxylate, alkylpolyglycoside, alkyldimethylamineoxide, ethoxylated fatty acid monoethanolamide, fatty acid monoethanolamide, or polyhydroxy alkyl fatty acid amide (as described for example in WO92/06154, which is incorporated herein by reference).

A detergent composition herein can optionally comprise one or more detergent builders or builder systems. In some aspects, oxidized alpha-1,3-glucan can be included as a co-builder; oxidized alpha-1,3-glucan compounds for use herein are disclosed in U.S. Patent Appl. Publ. No. 2015/0259439. In some aspects incorporating at least one builder, the cleaning compositions comprise at least about 1%, from about 3% to about 60%, or even from about 5% to about 40%, builder by weight of the composition. Examples of builders include alkali metal, ammonium and alkanolammonium salts of polyphosphates, alkali metal silicates, alkaline earth and alkali metal carbonates, aluminosilicates, polycarboxylate compounds, ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof. Additional examples of a detergent builder or complexing agent include zeolite, diphosphate, triphosphate, phosphonate, citrate, nitrilotriacetic acid (NTA), ethylenediaminetetraacetic acid (EDTA), diethylenetriaminepentaacetic acid (DTMPA), alkyl- or alkenylsuccinic acid, soluble silicates or layered silicates (e.g., SKS-6 from Hoechst).

In some embodiments, builders form water-soluble hardness ion complexes (e.g., sequestering builders), such as citrates and polyphosphates (e.g., sodium tripolyphosphate and sodium tripolyphospate hexahydrate, potassium tripolyphosphate, and mixed sodium and potassium tripolyphosphate, etc.). It is contemplated that any suitable builder will find use in the present disclosure, including those known in the art (See, e.g., EP2100949). In some embodiments, suitable builders can include phosphate builders and non-phosphate builders. In some embodiments, a builder is a phosphate builder. In some embodiments, a builder is a non-phosphate builder. A builder can be used in a level of from 0.1% to 80%, or from 5% to 60%, or from 10% to 50%, by weight of the composition. In some embodiments, the product comprises a mixture of phosphate and non-phosphate builders. Suitable phosphate builders include mono-phosphates, di-phosphates, tri-polyphosphates or oligomeric-polyphosphates, including the alkali metal salts of these compounds, including the sodium salts. In some embodiments, a builder can be sodium tripolyphosphate (STPP). Additionally, the composition can comprise carbonate and/or citrate, preferably citrate that helps to achieve a neutral pH composition. Other suitable non-phosphate builders include homopolymers and copolymers of polycarboxylic acids and their partially or completely neutralized salts, monomeric polycarboxylic acids and hydroxycarboxylic acids and their salts. In some embodiments, salts of the above mentioned compounds include ammonium and/or alkali metal salts, i.e., lithium, sodium, and potassium salts, including sodium salts. Suitable polycarboxylic acids include acyclic, alicyclic, hetero-cyclic and aromatic carboxylic acids, wherein in some embodiments, they can contain at least two carboxyl groups which are in each case separated from one another by, in some instances, no more than two carbon atoms.

A detergent composition herein can comprise at least one chelating agent. Suitable chelating agents include, but are not limited to copper, iron and/or manganese chelating agents and mixtures thereof. In embodiments in which at least one chelating agent is used, the composition comprises from about 0.1% to about 15%, or even from about 3.0% to about 10%, chelating agent by weight of the composition.

A detergent composition herein can comprise at least one deposition aid. Suitable deposition aids include, but are not limited to, polyethylene glycol, polypropylene glycol, polycarboxylate, soil release polymers such as polytelephthalic acid, clays such as kaolinite, montmorillonite, atapulgite, illite, bentonite, halloysite, and mixtures thereof.

A detergent composition herein can comprise one or more dye transfer-inhibiting agents. Suitable polymeric dye transfer-inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. Additional dye transfer-inhibiting agents include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof; chelating agents examples of which include ethylene-diamine-tetraacetic acid (EDTA); diethylene triamine penta methylene phosphonic acid (DTPMP); hydroxy-ethane diphosphonic acid (HEDP); ethylenediamine N,N′-disuccinic acid (EDDS); methyl glycine diacetic acid (MGDA); diethylene triamine penta acetic acid (DTPA); propylene diamine tetraacetic acid (PDT A); 2-hydroxypyridine-N-oxide (HPNO); or methyl glycine diacetic acid (MGDA); glutamic acid N,N-diacetic acid (N,N-dicarboxymethyl glutamic acid tetrasodium salt (GLDA); nitrilotriacetic acid (NTA); 4,5-dihydroxy-m-benzenedisulfonic acid; citric acid and any salts thereof; N-hydroxyethyl ethylenediaminetriacetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP) and derivatives thereof, which can be used alone or in combination with any of the above. In embodiments in which at least one dye transfer-inhibiting agent is used, a composition herein may comprise from about 0.0001% to about 10%, from about 0.01% to about 5%, or even from about 0.1% to about 3%, by weight of the composition.

A detergent composition herein can comprise silicates. In some of these embodiments, sodium silicates (e.g., sodium disilicate, sodium metasilicate, and/or crystalline phyllosilicates) find use. In some embodiments, silicates are present at a level of from about 1% to about 20% by weight of the composition. In some embodiments, silicates are present at a level of from about 5% to about 15% by weight of the composition.

A detergent composition herein can comprise dispersants. Suitable water-soluble organic materials include, but are not limited to the homo- or co-polymeric acids or their salts, in which the polycarboxylic acid comprises at least two carboxyl radicals separated from each other by not more than two carbon atoms.

A detergent composition herein may additionally comprise one or more enzymes as disclosed above, for example. In some aspects, a detergent composition can comprise one or more enzymes, each at a level from about 0.00001% to about 10% by weight of the composition and the balance of cleaning adjunct materials by weight of composition. In some other aspects, a detergent composition can also comprise each enzyme at a level of about 0.0001% to about 10%, about 0.001% to about 5%, about 0.001% to about 2%, or about 0.005% to about 0.5%, by weight of the composition. Enzymes comprised in a detergent composition herein may be stabilized using conventional stabilizing agents, e.g., a polyol such as propylene glycol or glycerol; a sugar or sugar alcohol; lactic acid; boric acid or a boric acid derivative (e.g., an aromatic borate ester).

A detergent composition in some aspects may comprise one or more other types of polymer in addition to a glucan ester derivative as disclosed herein. Examples of other types of polymers useful herein include carboxymethyl cellulose (CMC), dextran, poly (vinylpyrrolidone) (PVP), polyethylene glycol (PEG), poly(vinyl alcohol) (PVA), polycarboxylates such as polyacrylates, maleic/acrylic acid copolymers and lauryl methacrylate/acrylic acid copolymers.

A detergent composition herein may contain a bleaching system. For example, a bleaching system can comprise an H₂O₂ source such as perborate or percarbonate, which may be combined with a peracid-forming bleach activator such as tetraacetylethylenediamine (TAED) or nonanoyloxybenzenesulfonate (NOBS).

Alternatively, a bleaching system may comprise peroxyacids (e.g., amide, imide, or sulfone type peroxyacids). Alternatively still, a bleaching system can be an enzymatic bleaching system comprising perhydrolase, for example, such as the system described in WO2005/056783.

A detergent composition herein may also contain conventional detergent ingredients such as fabric conditioners, clays, foam boosters, suds suppressors, anti-corrosion agents, soil-suspending agents, anti-soil redeposition agents, dyes, bactericides, tarnish inhibiters, optical brighteners, or perfumes. The pH of a detergent composition herein (measured in aqueous solution at use concentration) is usually neutral or alkaline (e.g., pH of about 7.0 to about 11.0).

Examples of suitable anti-redeposition and/or clay soil removal agents for a fabric care product herein include polyethoxy zwitterionic surfactants, water-soluble copolymers of acrylic or methacrylic acid with acrylic or methacrylic acid-ethylene oxide condensates (e.g., U.S. Pat. No. 3,719,647), cellulose derivatives such as carboxymethylcellulose and hydroxypropylcellulose (e.g., U.S. Pat. Nos. 3,597,416 and 3,523,088), and mixtures comprising nonionic alkyl polyethoxy surfactant, polyethoxy alkyl quaternary cationic surfactant and fatty amide surfactant (e.g., U.S. Pat. No. 4,228,044). Non-limiting examples of other suitable anti-redeposition and clay soil removal agents are disclosed in U.S. Pat. Nos. 4,597,898 and 4,891,160, and International Patent Appl. Publ. No. WO95/32272, all of which are incorporated herein by reference.

Particular forms of detergent compositions that can be adapted for purposes herein are disclosed in, for example, US20090209445A1, US20100081598A1, U.S. Pat. No. 7,001,878B2, EP1504994B1, WO2001085888A2, WO2003089562A1, WO2009098659A1, WO2009098660A1, WO2009112992A1, WO2009124160A1, WO2009152031A1, WO2010059483A1, WO2010088112A1, WO2010090915A1, WO2010135238A1, WO2011094687A1, WO2011094690A1, WO2011127102A1, WO2011163428A1, WO2008000567A1, WO2006045391A1, WO2006007911A1, WO2012027404A1, EP1740690B1, WO2012059336A1, U.S. Pat. No. 6,730,646B1, WO2008087426A1, WO2010116139A1, and WO2012104613A1, all of which are incorporated herein by reference.

Laundry detergent compositions herein can optionally be heavy duty (all purpose) laundry detergent compositions. Exemplary heavy duty laundry detergent compositions comprise a detersive surfactant (10%-40% wt/wt), including an anionic detersive surfactant (selected from a group of linear or branched or random chain, substituted or unsubstituted alkyl sulphates, alkyl sulphonates, alkyl alkoxylated sulphate, alkyl phosphates, alkyl phosphonates, alkyl carboxylates, and/or mixtures thereof), and optionally non-ionic surfactant (selected from a group of linear or branched or random chain, substituted or unsubstituted alkyl alkoxylated alcohol, e.g., C8-C18 alkyl ethoxylated alcohols and/or C6-C12 alkyl phenol alkoxylates), where the weight ratio of anionic detersive surfactant (with a hydrophilic index (HIc) of from 6.0 to 9) to non-ionic detersive surfactant is greater than 1:1. Suitable detersive surfactants also include cationic detersive surfactants (selected from a group of alkyl pyridinium compounds, alkyl quaternary ammonium compounds, alkyl quaternary phosphonium compounds, alkyl ternary sulphonium compounds, and/or mixtures thereof); zwitterionic and/or amphoteric detersive surfactants (selected from a group of alkanolamine sulpho-betaines); ampholytic surfactants; semi-polar non-ionic surfactants and mixtures thereof.

A detergent herein such as a heavy duty laundry detergent composition may optionally include, a surfactancy boosting polymer consisting of amphiphilic alkoxylated grease cleaning polymers (selected from a group of alkoxylated polymers having branched hydrophilic and hydrophobic properties, such as alkoxylated polyalkylenimines in the range of 0.05 wt %-10 wt %) and/or random graft polymers (typically comprising of hydrophilic backbone comprising monomers selected from the group consisting of: unsaturated C₁-C₆ carboxylic acids, ethers, alcohols, aldehydes, ketones, esters, sugar units, alkoxy units, maleic anhydride, saturated polyalcohols such as glycerol, and mixtures thereof; and hydrophobic side chain(s) selected from the group consisting of: C4-C₂₅ alkyl group, polypropylene, polybutylene, vinyl ester of a saturated C₁-C₆ mono-carboxylic acid, C₁-C₆ alkyl ester of acrylic or methacrylic acid, and mixtures thereof.

A detergent herein such as a heavy duty laundry detergent composition may optionally include additional polymers such as soil release polymers (include anionically end-capped polyesters, for example SRP1, polymers comprising at least one monomer unit selected from saccharide, dicarboxylic acid, polyol and combinations thereof, in random or block configuration, ethylene terephthalate-based polymers and co-polymers thereof in random or block configuration, for example REPEL-O-TEX SF, SF-2 AND SRP6, TEXCARE SRA100, SRA300, SRN100, SRN170, SRN240, SRN300 AND SRN325, MARLOQUEST SL), anti-redeposition agent(s) herein (0.1 wt % to 10 wt %), include carboxylate polymers, such as polymers comprising at least one monomer selected from acrylic acid, maleic acid (or maleic anhydride), fumaric acid, itaconic acid, aconitic acid, mesaconic acid, citraconic acid, methylenemalonic acid, and any mixture thereof, vinylpyrrolidone homopolymer, and/or polyethylene glycol, molecular weight in the range of from 500 to 100,000 Da); and polymeric carboxylate (such as maleate/acrylate random copolymer or polyacrylate homopolymer).

A detergent herein such as a heavy duty laundry detergent composition may optionally further include saturated or unsaturated fatty acids, preferably saturated or unsaturated C12-C24 fatty acids (0 wt % to 10 wt %); deposition aids (examples for which include polysaccharides, cellulosic polymers, poly diallyl dimethyl ammonium halides (DADMAC), and co-polymers of DAD MAC with vinyl pyrrolidone, acrylamides, imidazoles, imidazolinium halides, and mixtures thereof, in random or block configuration, cationic guar gum, cationic starch, cationic polyacrylamides, and mixtures thereof.

A detergent herein such as a heavy duty laundry detergent composition may optionally further include at least one dye transfer-inhibiting agent, examples of which are described above.

A detergent herein such as a heavy duty laundry detergent composition may optionally include silicone or fatty-acid based suds suppressors; hueing dyes, calcium and magnesium cations, visual signaling ingredients, anti-foam (0.001 wt % to about 4.0 wt %), and/or a structurant/thickener (0.01 wt % to 5 wt %) selected from the group consisting of diglycerides and triglycerides, ethylene glycol distearate, microcrystalline cellulose, microfiber cellulose, biopolymers, xanthan gum, gellan gum, and mixtures thereof). A structurant can also be referred to as a structural agent.

A detergent herein can be in the form of a heavy duty dry/solid laundry detergent composition, for example. Such a detergent may include: (i) a detersive surfactant, such as any anionic detersive surfactant disclosed herein, any non-ionic detersive surfactant disclosed herein, any cationic detersive surfactant disclosed herein, any zwitterionic and/or amphoteric detersive surfactant disclosed herein, any ampholytic surfactant, any semi-polar non-ionic surfactant, and mixtures thereof; (ii) a builder, such as any phosphate-free builder (e.g., zeolite builders in the range of 0 wt % to less than 10 wt %), any phosphate builder (e.g., sodium tri-polyphosphate in the range of 0 wt % to less than 10 wt %), citric acid, citrate salts and nitrilotriacetic acid, any silicate salt (e.g., sodium or potassium silicate or sodium meta-silicate in the range of 0 wt % to less than 10 wt %); any carbonate salt (e.g., sodium carbonate and/or sodium bicarbonate in the range of 0 wt % to less than 80 wt %), and mixtures thereof; (iii) a bleaching agent, such as any photobleach (e.g., sulfonated zinc phthalocyanines, sulfonated aluminum phthalocyanines, xanthenes dyes, and mixtures thereof), any hydrophobic or hydrophilic bleach activator (e.g., dodecanoyl oxybenzene sulfonate, decanoyl oxybenzene sulfonate, decanoyl oxybenzoic acid or salts thereof, 3,5,5-trimethy hexanoyl oxybenzene sulfonate, tetraacetyl ethylene diamine-TAED, nonanoyloxybenzene sulfonate-NOBS, nitrile quats, and mixtures thereof), any source of hydrogen peroxide (e.g., inorganic perhydrate salts, examples of which include mono or tetra hydrate sodium salt of perborate, percarbonate, persulfate, perphosphate, or persilicate), any preformed hydrophilic and/or hydrophobic peracids (e.g., percarboxylic acids and salts, percarbonic acids and salts, perimidic acids and salts, peroxymonosulfuric acids and salts, and mixtures thereof); and/or (iv) any other components such as a bleach catalyst (e.g., imine bleach boosters examples of which include iminium cations and polyions, iminium zwitterions, modified amines, modified amine oxides, N-sulphonyl imines, N-phosphonyl imines, N-acyl imines, thiadiazole dioxides, perfluoroimines, cyclic sugar ketones, and mixtures thereof), and a metal-containing bleach catalyst (e.g., copper, iron, titanium, ruthenium, tungsten, molybdenum, or manganese cations along with an auxiliary metal cations such as zinc or aluminum and a sequestrate such as EDTA, ethylenediaminetetra(methylenephosphonic acid).

A detergent herein such as that for fabric care (e.g., laundry) can be comprised in a unit dose (e.g., sachet or pouch), for example. A unit dose form can comprise a water-soluble outer film that completely envelopes a liquid or solid detergent composition. A unit dose can comprise a single compartment, or at least two, three, or more (multiple) compartments. Multiple compartments can be arranged in a superposed orientation or a side-by-side orientation. A unit dose herein is typically a closed structure of any form/shape suitable for holding and protecting its contents without allowing contents release prior to contact with water.

In some aspects, a composition comprising at least one glucan ester derivative herein can be in the form of, or comprise, a fabric softener (liquid fabric softener). An example of such a composition is a rinse used in laundering a fabric-comprising material herein typically following cleaning of the fabric-comprising material with a laundry detergent composition (e.g., laundry rinse such as used in a laundry rinse cycle in a washing machine). The concentration of a glucan ester derivative in a composition comprising fabric softener (e.g., a rinse) can be about, or at least about, 20, 30, 40, 50, 60, 70, 80, 20-80, 20-70, 20-60, 30-80, 30-70, 30-60, 40-80, 40-70, or 40-60 ppm, for example. The concentration of a fabric softener in a composition (e.g., a rinse) can be about, or at least about, 50, 75, 100, 150, 200, 300, 400, 500, 600, 50-600, 50-500, 50-400, 50-300, 50-200, 100-600, 100-500, 100-400, 100-300, 100-200, 10-600, 50-500, 50-400, 50-300, 50-200, 200-600, 200-500, 200-400, or 200-300 ppm, for example. Fabric softener concentration can be based on the total fabric softener composition added (not necessarily based on an individual component of the fabric softener), or based on one or more fabric softening agents(s) in the fabric softener formulation. A fabric softener herein can further comprise, for example, one or more of a fabric softening agent (e.g., diethyl ester dimethyl ammonium chloride), anti-static agent, perfume, wetting agent, viscosity modifier (e.g., calcium chloride), pH buffer/buffering agent (e.g., formic acid), antimicrobial agent, anti-oxidant, radical scavenger (e.g., ammonium chloride), chelant/builder (e.g., diethylenetriamine pentaacetate), anti-foaming agent/lubricant (e.g., polydimethylsiloxane), preservative (e.g., benzisothiazolinone) and colorant. In some aspects, a fabric softener can further comprise one or more of a fabric softening agent, viscosity modifier, pH buffer/buffering agent, radical scavenger, chelant/builder and anti-foaming agent/lubricant. A fabric softener can be perfume-free and/or dye-free, or have less than about 0.1 wt % of a perfume and/or dye in some aspects. In some aspects, a fabric softener that can be adapted for use herein can be as disclosed in any of U.S. Patent Appl. Publ. Nos. 2014/0366282, 2001/0018410, 2006/0058214, 2021/0317384, or 2006/0014655, or Int. Patent Appl. Publ. Nos. WO2007/078782, WO1998/016538, WO1998/012293, WO1998007920, WO2000/070004, WO2009/146981, WO2000/70005, or WO2013087366, which are incorporated herein by reference. Some brands of fabric softeners that can be adapted for use herein, if desired, include DOWNY, DOWNY ULTRA, DOWNY INFUSIONS, ALL, SNUGGLE, LENOR and GAIN. A liquid fabric softener product (e.g., as it exists before being used in a laundry rinse cycle) can be formulated to include one or more glucan ester derivatives in some aspects. A fabric softener in some aspects can be in a unit dose, such as disclosed herein for a detergent.

Compositions disclosed herein comprising at least one glucan ester derivative can be in the form of, or comprise, a dishwashing detergent composition, for example. Examples of dishwashing detergents include automatic dishwashing detergents (typically used in dishwasher machines) and hand-washing dish detergents. A dishwashing detergent composition can be in any dry or liquid/aqueous form as disclosed herein, for example. Components that may be included in some aspects of a dishwashing detergent composition include, for example, one or more of a phosphate; oxygen- or chlorine-based bleaching agent; non-ionic surfactant; alkaline salt (e.g., metasilicates, alkali metal hydroxides, sodium carbonate); any active enzyme disclosed herein; anti-corrosion agent (e.g., sodium silicate); anti-foaming agent; additives to slow down the removal of glaze and patterns from ceramics; perfume; anti-caking agent (in granular detergent); starch (in tablet-based detergents); gelling agent (in liquid/gel based detergents); and/or sand (powdered detergents).

Dishwashing detergents such as an automatic dishwasher detergent or liquid dishwashing detergent can comprise (i) a non-ionic surfactant, including any ethoxylated non-ionic surfactant, alcohol alkoxylated surfactant, epoxy-capped poly (oxyalkylated) alcohol, or amine oxide surfactant present in an amount from 0 to 10 wt %; (ii) a builder, in the range of about 5-60 wt %, including any phosphate builder (e.g., mono-phosphates, di-phosphates, tri-polyphosphates, other oligomeric-polyphosphates, sodium tripolyphosphate-STPP), any phosphate-free builder (e.g., amino acid-based compounds including methyl-glycine-diacetic acid [MGDA] and salts or derivatives thereof, glutamic-N, N-diacetic acid [GLDA] and salts or derivatives thereof, iminodisuccinic acid (IDS) and salts or derivatives thereof, carboxy methyl inulin and salts or derivatives thereof, nitrilotriacetic acid [NTA], diethylene triamine penta acetic acid [DTPA], B-alaninediacetic acid [B-ADA] and salts thereof), homopolymers and copolymers of poly-carboxylic acids and partially or completely neutralized salts thereof, monomeric polycarboxylic acids and hydroxycarboxylic acids and salts thereof in the range of 0.5 wt % to 50 wt %, or sulfonated/carboxylated polymers in the range of about 0.1 wt % to about 50 wt %; (iii) a drying aid in the range of about 0.1 wt % to about 10 wt % (e.g., polyesters, especially anionic polyesters, optionally together with further monomers with 3 to 6 functionalities-typically acid, alcohol or ester functionalities which are conducive to polycondensation, polycarbonate-, polyurethane- and/or polyurea-polyorganosiloxane compounds or precursor compounds thereof, particularly of the reactive cyclic carbonate and urea type); (iv) a silicate in the range from about 1 wt % to about 20 wt % (e.g., sodium or potassium silicates such as sodium disilicate, sodium meta-silicate and crystalline phyllosilicates); (v) an inorganic bleach (e.g., perhydrate salts such as perborate, percarbonate, perphosphate, persulfate and persilicate salts) and/or an organic bleach (e.g., organic peroxyacids such as diacyl- and tetraacylperoxides, especially diperoxydodecanedioic acid, diperoxytetradecanedioic acid, and diperoxyhexadecanedioic acid); (vi) a bleach activator (e.g., organic peracid precursors in the range from about 0.1 wt % to about 10 wt %) and/or bleach catalyst (e.g., manganese triazacyclononane and related complexes; Co, Cu, Mn, and Fe bispyridylamine and related complexes; and pentamine acetate cobalt (III) and related complexes); (vii) a metal care agent in the range from about 0.1 wt % to 5 wt % (e.g., benzatriazoles, metal salts and complexes, and/or silicates); (viii) a glass corrosion inhibitor in the range of about 0.1 wt % to 5 wt % (e.g., a salt and/or complex of magnesium, zinc, or bismuth); and/or (ix) any active enzyme disclosed herein in the range from about 0.01 to 5.0 mg of active enzyme per gram of automatic dishwashing detergent composition, and an enzyme stabilizer component (e.g., oligosaccharides, polysaccharides, and inorganic divalent metal salts). In some aspects, a dishwashing detergent ingredient or entire composition (but adapted accordingly to comprise a glucan ester derivative herein) can be as disclosed in U.S. Pat. No. 8,575,083 or 9,796,951, or U.S. Pat. Appl. Publ. No. 2017/0044468, which are each incorporated herein by reference.

A detergent herein such as that for dish care can be comprised in a unit dose (e.g., sachet or pouch) (e.g., water-soluble unit dose article), for example, and can be as described above for a fabric care detergent, but rather comprise a suitable dish detergent composition.

It is believed that numerous commercially available detergent formulations can be adapted to include a glucan ester derivative as disclosed herein. Examples of commercially available detergent formulations include PUREX® ULTRAPACKS (Henkel), FINISH® QUANTUM (Reckitt Benckiser), CLOROX™ 2 PACKS (Clorox), OXICLEAN MAX FORCE POWER PAKS (Church & Dwight), TIDE® STAIN RELEASE, CASCADE® ACTIONPACS, and TIDE® PODS™ (Procter & Gamble).

Compositions disclosed herein comprising at least one glucan ester derivative can be in the form of, or comprise, an oral care composition, for example. Examples of oral care compositions include dentifrices, toothpaste, mouth wash, mouth rinse, chewing gum, and edible strips that provide some form of oral care (e.g., treatment or prevention of cavities [dental caries], gingivitis, plaque, tartar, and/or periodontal disease). An oral care composition can also be for treating an “oral surface”, which encompasses any soft or hard surface within the oral cavity including surfaces of the tongue, hard and soft palate, buccal mucosa, gums and dental surfaces. A “dental surface” herein is a surface of a natural tooth or a hard surface of artificial dentition including a crown, cap, filling, bridge, denture, or dental implant, for example.

An oral care composition herein can comprise about 0.01-15.0 wt % (e.g., ~0.1-10 wt % or ~0.1-5.0 wt %, ~0.1-2.0 wt %) of a glucan ester derivative as disclosed herein, for example. A glucan ester derivative comprised in an oral care composition can sometimes be provided therein as a thickening agent and/or dispersion agent, which may be useful to impart a desired consistency and/or mouth feel to the composition. One or more other thickening or dispersion agents can also be provided in an oral care composition herein, such as a carboxyvinyl polymer, carrageenan (e.g., L-carrageenan), natural gum (e.g., karaya, xanthan, gum arabic, tragacanth), colloidal magnesium aluminum silicate, or colloidal silica, for example.

An oral care composition herein may be a toothpaste or other dentifrice, for example. Such compositions, as well as any other oral care composition herein, can additionally comprise, without limitation, one or more of an anticaries agent, antimicrobial or antibacterial agent, anticalculus or tartar control agent, surfactant, abrasive, pH-modifying agent, foam modulator, humectant, flavorant, sweetener, pigment/colorant, whitening agent, and/or other suitable components. Examples of oral care compositions to which a glucan ester derivative herein can be added are disclosed in U.S. Patent Appl. Publ. Nos. 2006/0134025, 2002/0022006 and 2008/0057007, which are incorporated herein by reference.

An anticaries agent herein can be an orally acceptable source of fluoride ions. Suitable sources of fluoride ions include fluoride, monofluorophosphate and fluorosilicate salts as well as amine fluorides, including olaflur (N′-octadecyltrimethylendiamine-N,N,N′-tris (2-ethanol)-dihydrofluoride), for example. An anticaries agent can be present in an amount providing a total of about 100-20000 ppm, about 200-5000 ppm, or about 500-2500 ppm, fluoride ions to the composition, for example. In oral care compositions in which sodium fluoride is the sole source of fluoride ions, an amount of about 0.01-5.0 wt %, about 0.05-1.0 wt %, or about 0.1-0.5 wt %, sodium fluoride can be present in the composition, for example.

An antimicrobial or antibacterial agent suitable for use in an oral care composition herein includes, for example, phenolic compounds (e.g., 4-allylcatechol; p-hydroxybenzoic acid esters such as benzylparaben, butylparaben, ethylparaben, methylparaben and propylparaben; 2-benzylphenol; butylated hydroxyanisole; butylated hydroxytoluene; capsaicin; carvacrol; creosol; eugenol; guaiacol; halogenated bisphenolics such as hexachlorophene and bromochlorophene; 4-hexylresorcinol; 8-hydroxyquinoline and salts thereof; salicylic acid esters such as menthyl salicylate, methyl salicylate and phenyl salicylate; phenol; pyrocatechol; salicylanilide; thymol; halogenated diphenylether compounds such as triclosan and triclosan monophosphate), copper (II) compounds (e.g., copper (II) chloride, fluoride, sulfate and hydroxide), zinc ion sources (e.g., zinc acetate, citrate, gluconate, glycinate, oxide, and sulfate), phthalic acid and salts thereof (e.g., magnesium monopotassium phthalate), hexetidine, octenidine, sanguinarine, benzalkonium chloride, domiphen bromide, alkylpyridinium chlorides (e.g. cetylpyridinium chloride, tetradecylpyridinium chloride, N-tetradecyl-4-ethylpyridinium chloride), iodine, sulfonamides, bisbiguanides (e.g., alexidine, chlorhexidine, chlorhexidine digluconate), piperidino derivatives (e.g., delmopinol, octapinol), magnolia extract, grapeseed extract, rosemary extract, menthol, geraniol, citral, eucalyptol, antibiotics (e.g., augmentin, amoxicillin, tetracycline, doxycycline, minocycline, metronidazole, neomycin, kanamycin, clindamycin), and/or any antibacterial agents disclosed in U.S. Pat. No. 5,776,435, which is incorporated herein by reference. One or more antimicrobial agents can optionally be present at about 0.01-10 wt % (e.g., 0.1-3 wt %), for example, in the disclosed oral care composition.

An anticalculus or tartar control agent suitable for use in an oral care composition herein includes, for example, phosphates and polyphosphates (e.g., pyrophosphates), polyaminopropanesulfonic acid (AMPS), zinc citrate trihydrate, polypeptides (e.g., polyaspartic and polyglutamic acids), polyolefin sulfonates, polyolefin phosphates, diphosphonates (e.g., azacycloalkane-2,2-diphosphonates such as azacycloheptane-2,2-diphosphonic acid), N-methyl azacyclopentane-2,3-diphosphonic acid, ethane-1-hydroxy-1, 1-diphosphonic acid (EHDP), ethane-1-amino-1,1-diphosphonate, and/or phosphonoalkane carboxylic acids and salts thereof (e.g., their alkali metal and ammonium salts). Useful inorganic phosphate and polyphosphate salts include, for example, monobasic, dibasic and tribasic sodium phosphates, sodium tripolyphosphate, tetrapolyphosphate, mono-, di-, tri- and tetra-sodium pyrophosphates, disodium dihydrogen pyrophosphate, sodium trimetaphosphate, sodium hexametaphosphate, or any of these in which sodium is replaced by potassium or ammonium. Other useful anticalculus agents in certain embodiments include anionic polycarboxylate polymers (e.g., polymers or copolymers of acrylic acid, methacrylic, and maleic anhydride such as polyvinyl methyl ether/maleic anhydride copolymers). Still other useful anticalculus agents include sequestering agents such as hydroxycarboxylic acids (e.g., citric, fumaric, malic, glutaric and oxalic acids and salts thereof) and aminopolycarboxylic acids (e.g., EDTA). One or more anticalculus or tartar control agents can optionally be present at about 0.01-50 wt % (e.g., about 0.05-25 wt % or about 0.1-15 wt %), for example, in the disclosed oral care composition.

A surfactant suitable for use in an oral care composition herein may be anionic, non-ionic, or amphoteric, for example. Suitable anionic surfactants include, without limitation, water-soluble salts of C₈₋₂₀ alkyl sulfates, sulfonated monoglycerides of C₈₋₂₀ fatty acids, sarcosinates, and taurates. Examples of anionic surfactants include sodium lauryl sulfate, sodium coconut monoglyceride sulfonate, sodium lauryl sarcosinate, sodium lauryl isoethionate, sodium laureth carboxylate and sodium dodecyl benzenesulfonate. Suitable non-ionic surfactants include, without limitation, poloxamers, polyoxyethylene sorbitan esters, fatty alcohol ethoxylates, alkylphenol ethoxylates, tertiary amine oxides, tertiary phosphine oxides, and dialkyl sulfoxides. Suitable amphoteric surfactants include, without limitation, derivatives of C₈₋₂₀ aliphatic secondary and tertiary amines having an anionic group such as a carboxylate, sulfate, sulfonate, phosphate or phosphonate. An example of a suitable amphoteric surfactant is cocoamidopropyl betaine. One or more surfactants are optionally present in a total amount of about 0.01-10 wt % (e.g., about 0.05-5.0 wt % or about 0.1-2.0 wt %), for example, in the disclosed oral care composition.

An abrasive suitable for use in an oral care composition herein may include, for example, silica (e.g., silica gel, hydrated silica, precipitated silica), alumina, insoluble phosphates, calcium carbonate, and resinous abrasives (e.g., a urea-formaldehyde condensation product). Examples of insoluble phosphates useful as abrasives herein are orthophosphates, polymetaphosphates and pyrophosphates, and include dicalcium orthophosphate dihydrate, calcium pyrophosphate, beta-calcium pyrophosphate, tricalcium phosphate, calcium polymetaphosphate and insoluble sodium polymetaphosphate. One or more abrasives are optionally present in a total amount of about 5-70 wt % (e.g., about 10-56 wt % or about 15-30 wt %), for example, in the disclosed oral care composition. The average particle size of an abrasive in certain embodiments is about 0.1-30 microns (e.g., about 1-20 microns or about 5-15 microns).

An oral care composition in certain embodiments may comprise at least one pH-modifying agent. Such agents may be selected to acidify, make more basic, or buffer the pH of a composition to a pH range of about 2-10 (e.g., pH ranging from about 2-8, 3-9, 4-8, 5-7, 6-10, or 7-9). Examples of pH-modifying agents useful herein include, without limitation, carboxylic, phosphoric and sulfonic acids; acid salts (e.g., monosodium citrate, disodium citrate, monosodium malate); alkali metal hydroxides (e.g. sodium hydroxide, carbonates such as sodium carbonate, bicarbonates, sesquicarbonates); borates; silicates; phosphates (e.g., monosodium phosphate, trisodium phosphate, pyrophosphate salts); and imidazole.

A foam modulator suitable for use in an oral care composition herein may be a polyethylene glycol (PEG), for example. High molecular weight PEGs are suitable, including those having an average molecular weight of about 200000-7000000 (e.g., about 500000-5000000 or about 1000000-2500000), for example. One or more PEGs are optionally present in a total amount of about 0.1-10 wt % (e.g. about 0.2-5.0 wt % or about 0.25-2.0 wt %), for example, in the disclosed oral care composition.

An oral care composition in certain embodiments may comprise at least one humectant. A humectant in certain embodiments may be a polyhydric alcohol such as glycerin, sorbitol, xylitol, or a low molecular weight PEG. Most suitable humectants also may function as a sweetener herein. One or more humectants are optionally present in a total amount of about 1.0-70 wt % (e.g., about 1.0-50 wt %, about 2-25 wt %, or about 5-15 wt %), for example, in the disclosed oral care composition.

A natural or artificial sweetener may optionally be comprised in an oral care composition herein. Examples of suitable sweeteners include dextrose, sucrose, maltose, dextrin, invert sugar, mannose, xylose, ribose, fructose, levulose, galactose, corn syrup (e.g., high fructose corn syrup or corn syrup solids), partially hydrolyzed starch, hydrogenated starch hydrolysate, sorbitol, mannitol, xylitol, maltitol, isomalt, aspartame, neotame, saccharin and salts thereof, dipeptide-based intense sweeteners, and cyclamates. One or more sweeteners are optionally present in a total amount of about 0.005-5.0 wt %, for example, in the disclosed oral care composition.

A natural or artificial flavorant may optionally be comprised in an oral care composition herein. Examples of suitable flavorants include vanillin; sage; marjoram; parsley oil; spearmint oil; cinnamon oil; oil of wintergreen (methylsalicylate); peppermint oil; clove oil; bay oil; anise oil; eucalyptus oil; citrus oils; fruit oils; essences such as those derived from lemon, orange, lime, grapefruit, apricot, banana, grape, apple, strawberry, cherry, or pineapple; bean- and nut-derived flavors such as coffee, cocoa, cola, peanut, or almond; and adsorbed and encapsulated flavorants. Also encompassed within flavorants herein are ingredients that provide fragrance and/or other sensory effect in the mouth, including cooling or warming effects. Such ingredients include, without limitation, menthol, menthyl acetate, menthyl lactate, camphor, eucalyptus oil, eucalyptol, anethole, eugenol, cassia, oxanone, Irisone®, propenyl guaiethol, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), and menthone glycerol acetal (MGA). One or more flavorants are optionally present in a total amount of about 0.01-5.0 wt % (e.g., about 0.1-2.5 wt %), for example, in the disclosed oral care composition.

An oral care composition in certain embodiments may comprise at least one bicarbonate salt. Any orally acceptable bicarbonate can be used, including alkali metal bicarbonates such as sodium or potassium bicarbonate, and ammonium bicarbonate, for example. One or more bicarbonate salts are optionally present in a total amount of about 0.1-50 wt % (e.g., about 1-20 wt %), for example, in the disclosed oral care composition.

An oral care composition in certain embodiments may comprise at least one whitening agent and/or colorant. A suitable whitening agent is a peroxide compound such as any of those disclosed in U.S. Pat. No. 8,540,971, which is incorporated herein by reference. Suitable colorants herein include pigments, dyes, lakes and agents imparting a particular luster or reflectivity such as pearling agents, for example. Specific examples of colorants useful herein include talc; mica; magnesium carbonate; calcium carbonate; magnesium silicate; magnesium aluminum silicate; silica; titanium dioxide; zinc oxide; red, yellow, brown and black iron oxides; ferric ammonium ferrocyanide; manganese violet; ultramarine; titaniated mica; and bismuth oxychloride. One or more colorants are optionally present in a total amount of about 0.001-20 wt % (e.g., about 0.01-10 wt % or about 0.1-5.0 wt %), for example, in the disclosed oral care composition.

Additional components that can optionally be included in an oral composition herein include one or more enzymes (above), vitamins, and anti-adhesion agents, for example. Examples of vitamins useful herein include vitamin C, vitamin E, vitamin B5, and folic acid. Examples of suitable anti-adhesion agents include solbrol, ficin, and quorum-sensing inhibitors.

Additional examples of personal care, household care, and other products and ingredients herein can be any as disclosed in U.S. Pat. No. 8,796,196, which is incorporated herein by reference. Examples of personal care, household care, and other products and ingredients herein include perfumes, fragrances, insect repellents and insecticides, bubble-generating agents such as surfactants, pet insecticides, pet shampoos, disinfecting agents, hard surface (e.g., floor, tub/shower, sink, toilet bowl, door handle/panel, glass/window, car/automobile exterior or interior) treatment agents (e.g., cleaning, disinfecting, and/or coating agents), wipes and other non-woven materials, colorants, preservatives, antioxidants, emulsifiers, emollients, oils, medicaments, flavors, and suspending agents.

The present disclosure also concerns a method of treating a material. This method comprises contacting a material with an aqueous composition comprising at least one glucan ester derivative as disclosed herein.

A material contacted with an aqueous composition in a contacting method herein can comprise a fabric in some aspects. A fabric herein can comprise natural fibers, synthetic fibers, semi-synthetic fibers, or any combination thereof. A semi-synthetic fiber herein is produced using naturally occurring material that has been chemically derivatized, an example of which is rayon. Non-limiting examples of fabric types herein include fabrics made of (i) cellulosic fibers such as cotton (e.g., broadcloth, canvas, chambray, chenille, chintz, corduroy, cretonne, damask, denim, flannel, gingham, jacquard, knit, matelassé, oxford, percale, poplin, plissé, sateen, seersucker, sheers, terry cloth, twill, velvet), rayon (e.g., viscose, modal, lyocell), linen, and Tencel®; (ii) proteinaceous fibers such as silk, wool and related mammalian fibers; (iii) synthetic fibers such as polyester, acrylic, nylon, and the like; (iv) long vegetable fibers from jute, flax, ramie, coir, kapok, sisal, henequen, abaca, hemp and sunn; and (v) any combination of a fabric of (i)-(iv). Fabric comprising a combination of fiber types (e.g., natural and synthetic) include those with both a cotton fiber and polyester, for example. Materials/articles containing one or more fabrics herein include, for example, clothing, curtains, drapes, upholstery, carpeting, bed linens, bath linens, tablecloths, sleeping bags, tents, car interiors, etc. Other materials comprising natural and/or synthetic fibers include, for example, non-woven fabrics, paddings, paper, and foams.

An aqueous composition that is contacted with a fabric can be, for example, a fabric care composition (e.g., laundry detergent, fabric softener). Thus, a treatment method in certain embodiments can be considered a fabric care method or laundry method if employing a fabric care composition therein. A fabric care composition herein is contemplated to effect one or more of the following fabric care benefits (i.e., surface substantive effects): wrinkle removal, wrinkle reduction, wrinkle resistance, fabric wear reduction, fabric wear resistance, fabric pilling reduction, extended fabric life, fabric color maintenance, fabric color fading reduction, reduced dye transfer, fabric color restoration, fabric soiling reduction, fabric soil release, fabric shape retention, fabric smoothness enhancement, anti-redeposition of soil on fabric, anti-greying of laundry, improved fabric hand/handle, and/or fabric shrinkage reduction.

Examples of conditions (e.g., time, temperature, wash/rinse volumes) for conducting a fabric care method or laundry method herein are disclosed in WO1997/003161 and U.S. Pat. Nos. 4,794,661, 4,580,421 and 5,945,394, which are incorporated herein by reference. In other examples, a material comprising fabric can be contacted with an aqueous composition herein: (i) for at least about 5, 10, 20, 30, 40, 50, 60, 70, 80, 90, 100, 110, or 120 minutes; (ii) at a temperature of at least about 10, 15, 20, 25, 30, 35, 40, 45, 50, 55, 60, 65, 70, 75, 80, 85, 90, or 95° C. (e.g., for laundry wash or rinse: a “cold” temperature of about 15-30° C., a “warm” temperature of about 30-50° C., a “hot” temperature of about 50-95° C.); (iii) at a pH of about 2, 3, 4, 5, 6, 7, 8, 9, 10, 11, or 12 (e.g., pH range of about 2-12, or about 3-11); (iv) at a salt (e.g., NaCl) concentration of at least about 0.5, 1.0, 1.5, 2.0, 2.5, 3.0, 3.5, or 4.0 wt %; or any combination of (i)-(iv).

The contacting step in a fabric care method or laundry method can comprise any of washing, soaking, and/or rinsing steps, for example. Contacting a material or fabric in still further embodiments can be performed by any means known in the art, such as dissolving, mixing, shaking, spraying, treating, immersing, flushing, pouring on or in, combining, painting, coating, applying, affixing to, and/or communicating an effective amount of a glucan ester derivative herein with the fabric or material. In still further embodiments, contacting may be used to treat a fabric to provide a surface substantive effect. As used herein, the term “fabric hand” or “handle” refers to a person's tactile sensory response towards fabric which may be physical, physiological, psychological, social or any combination thereof. In one embodiment, the fabric hand may be measured using a PhabrOmeter® System for measuring relative hand value (available from Nu Cybertek, Inc. Davis, CA) (American Association of Textile Chemists and Colorists [AATCC test method “202-2012, Relative Hand Value of Textiles: Instrumental Method”]).

In some aspects of treating a material comprising fabric, a glucan ester derivative of the aqueous composition adsorbs to the fabric. This feature is believed to render a glucan ester derivative herein useful as an anti-redeposition agent and/or anti-greying agent in fabric care compositions (in addition to its viscosity-modifying effect, e.g.). An anti-redeposition agent or anti-greying agent herein helps keep soil from redepositing onto clothing in wash water after the soil has been removed. It is further contemplated that adsorption of a glucan ester derivative herein to a fabric enhances mechanical properties of the fabric in some aspects.

Adsorption of a glucan ester derivative to a fabric herein can be measured using a colorimetric technique (e.g., Dubois et al., 1956, Anal. Chem. 28:350-356; Zemljič et al., 2006, Lenzinger Berichte 85:68-76; both incorporated herein by reference), for example, or any other method known in the art.

Other materials that can be contacted in the above treatment method include surfaces that can be treated with a dish detergent (e.g., automatic dishwashing detergent or hand dish detergent). Examples of such materials include surfaces of dishes, glasses, pots, pans, baking dishes, utensils and flatware made from ceramic material, china, metal, glass, plastic (e.g., polyethylene, polypropylene, polystyrene, melamine, etc.) and wood (collectively referred to herein as “tableware”). Thus, the treatment method in certain embodiments can be considered a dishwashing method or tableware washing method, for example. Examples of conditions (e.g., time, temperature, wash volume) for conducting a dishwashing or tableware washing method herein are disclosed herein and in U.S. Pat. No. 8,575,083 and U.S. Pat. Appl. Publ. No. 2017/0044468, which are incorporated herein by reference. In some aspects, a tableware article can be contacted with an aqueous composition herein under a suitable set of conditions such as any of those disclosed above with regard to contacting a fabric-comprising material.

Other materials that can be contacted in the above treatment method include oral surfaces such as any soft or hard surface within the oral cavity including surfaces of the tongue, hard and soft palate, buccal mucosa, gums and dental surfaces (e.g., natural tooth or a hard surface of artificial dentition such as a crown, cap, filling, bridge, denture, or dental implant). Thus, a treatment method in certain embodiments can be considered an oral care method or dental care method, for example. Conditions (e.g., time, temperature) for contacting an oral surface with an aqueous composition herein should be suitable for the intended purpose of making such contact. Other surfaces that can be contacted in a treatment method also include a surface of the integumentary system such as skin, hair or nails (i.e., any keratin-comprising tissue or material).

Thus, some aspects of the present disclosure concern material (e.g., fabric, or a fiber-comprising product as disclosed herein, or any other material herein such as hair, skin, or other keratin-comprising material) that comprises a glucan ester derivative herein. Such material can be produced following a material treatment method as disclosed herein, for example. A material may comprise a glucan ester derivative in some aspects if the glucan ester derivative is adsorbed to, or otherwise in contact with (e.g., glucan ester comprised in a coating of the material), the surface of the material.

Some aspects of a method of treating a material herein further comprise a drying step, in which a material is dried after being contacted with the aqueous composition. A drying step can be performed directly after the contacting step, or following one or more additional steps that might follow the contacting step (e.g., drying of fabric, tableware, or hair after being rinsed, in water for example, following a wash in an aqueous composition herein). Drying can be performed by any of several means known in the art, such as air drying (e.g., ~20-25° C.), or at a temperature of at least about 30, 40, 50, 60, 70, 80, 90, 100, 120, 140, 160, 170, 175, 180, or 200° C., for example. A material that has been dried herein typically has less than 3, 2, 1, 0.5, or 0.1 wt % water comprised therein.

An aqueous composition used in a treatment method herein can be any aqueous composition disclosed herein. Examples of aqueous compositions include detergents (e.g., laundry detergent or dish detergent), fabric softeners, water-containing dentifrices such as toothpaste, and hair care products such as hair styling, hair cleaning, or hair conditioning products.

Some aspects herein regard a method of styling hair. Such a method can comprise, for example, at least steps (a) and (b), or steps (c) and (d), as follows:
(a) contacting (e.g., coating) hair with a composition comprising a glucan ester derivative herein, thereby providing treated hair (or coated hair), and
(b) putting the treated hair (or the coated hair) into a desired form; or
(c) putting hair into a desired form, and
(d) contacting (e.g., coating) the hair of step (c) with a composition comprising a glucan ester derivative herein, thereby providing treated hair (or coated hair); and
(e) optionally, removing solvent, if present, that was used to deliver the glucan ester derivative to the hair in step (a) or (d).
   Such a method can optionally be characterized as a hair styling method. Contacting in a hair styling method can be performed, for example, by applying/treating hair with a hairstyling composition herein (e.g., gel, mouse, spray) comprising at least one glucan ester derivative. Hair to be treated in a hair styling method, particularly in steps (a) or (d), typically can be wet or dry. Step (e) of removing solvent can be performed by drying, for example, such as by a drying method disclosed herein (e.g., air drying or blow drying, with either room temperature or heated air). Drying can be done with (or without) agitation of the treated hair, such as by combing or brushing while drying. Optionally, a styling method herein can comprise, after step (b) or step (d) (but before optional step [e]), a step of applying steam to the treated hair. Step (b) or (c) of putting hair into a desired form can be performed in some aspects by straightening, curling, or otherwise putting the hair into a form that is different from the form the hair was in as it existed before step (a), (b), or (c). Hair that is styled by a styling method herein can hold, optionally without the need to apply any device and/or further material to the styled hair (i.e., while in a free-standing state), the desired form for a period of at least 1, 2, 3, 4, 5, or more days, for example. Such style retention can be in conditions of dry air (e.g., relative humidity≤50%) or humid air (e.g., relative humidity over 50%), for example (typically for a period of time during which the styled hair is not washed or rinsed).

In some aspects, a material that can be treated with an aqueous composition (e.g., dispersion/emulsion) herein is a non-woven product. This treatment, which can involve application of an aqueous composition herein (at any concentration disclosed herein) typically followed by a drying step (e.g., air drying, heated drying, vacuum drying; drying temperature can be any suitable temperature disclosed herein, for example), can strengthen (i.e., act as a binder for) a non-woven product. In some aspects, a glucan ester derivative as presently disclosed can increase the dry or wet tensile strength (measured in N/5 cm) of a non-woven by about, or at least about, 1000%, 10000%, 100000%, or 1000000%, for example. Thus, further provided herein are non-woven products containing a binder/strengthening agent that comprises a glucan ester derivative of the present disclosure. In some aspects, the dry or wet tensile strength of a non-woven comprising a glucan ester derivative herein can be about, or at least about, 10, 15, 20, 25, 50, 75, 100, 125, 130, 135, 140, 145, 150, 10-150, 15-150, 20-150, 25-150, 10-140, 15-140, 20-140, or 25-140 N/5 cm. On a basis of the total weight of non-woven material and a glucan ester derivative in a non-woven product, the content of the glucan ester derivative therein can be about 1, 2, 5, 10, 15, 20, 25, 1-5, 1-10, 5-20, or 1-25 wt %. A non-woven product herein can be, for example, air-laid, dry-laid, wet-laid, carded, electrospun, spun-lace, spun-bond, or melt-blown. In some aspects, a non-woven product can be an abrasive or scouring sheet, agricultural covering, agricultural seed strip, apparel lining, automobile headliner or upholstery, bib, cheese wrap, civil engineering fabric, coffee filter, cosmetic remover or applicator, detergent pouch/sachet, fabric softener sheet, envelope, face mask, filter, garment bag, heat or electricity conductive fabric, household care wipe (e.g., for floor care, hard surface cleaning, pet care etc.), house wrap, hygiene product (e.g., sanitary pad/napkin, underpad), insulation, label, laundry aid, medical care or personal injury care product (e.g., bandage, cast padding or cover, dressing, pack, sterile overwrap, sterile packaging, surgical drape, surgical gown, swab), mop, napkin or paper towel, paper, personal wipe or baby wipe, reusable bag, roofing undercovering, table linen, tag, tea or coffee bag, upholstery, vacuum cleaning bag, or wallcovering. The fiber of a non-woven product can comprise cellulose and/or alpha-1,3-glucan in some aspects, or can comprise one or more other materials disclosed herein that can be used to form a fiber. Examples of non-woven products herein, non-woven product materials, and/or methods of production of non-woven products and materials, can be as disclosed in U.S. Pat. Appl. Publ. Nos. 2020/0370216, 2018/0282918, 2017/0167063, 2018/0320291, or 2010/0291213, which are each incorporated herein by reference.

A composition herein comprising at least one glucan ester derivative as presently disclosed can be a film or coating, for example. A film or coating can be a dried film or coating in some aspects, comprising less than about 3, 2, 1, 0.5, or 0.1 wt % water, for example. In some aspects, a film or coating can comprise about 20-40, 20-35, 20-30, 25-40, 25-35, or 25-30 wt % a glucan ester derivative herein, where the balance of material in the film or coating optionally is water, an aqueous solution, and/or a plasticizer. The amount a glucan ester derivative as presently disclosed in a film or coating herein can be about, or at least about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.9, or 100 wt %, for example. A film or coating herein can be produced, for example, by providing a layer of an aqueous dispersion or solution of a glucan ester derivative (e.g., about 5-30, 5-25, 5-20, 10-30, 10-25, or 10-20 wt % glucan ester) onto a surface/object/material, and then removing all of, or most of (≥90, 95, 98, 99 wt %), the water from the dispersion or solution, thereby producing a film or coating. Methodology similar to, or as disclosed in, U.S. Pat. Appl. Publ. No. 2018/0258590 (incorporated herein by reference) can be used to produce a film or coating, for example. The grammage of a coating comprising a glucan ester derivative herein on a substrate can be about, or at least about, 1, 2, 3, 4, 5, 6, 7 8 9 10, 11, 12, 1-12, 1-10, 1-8, 1-6, 1-5, 1-4, 1-3, or 1-2 gsm (grams per square meter), for example.

A film or coating herein can have a thickness of about, at least about, or up to about, 0.5, 0.6, 0.7, 0.8, 0.9, 1.0, 1.1, 1.2, 1.3, 1.4, 1.5, 2, 2.5, 5, 7.5, 10, 15.5, 15, 17.5, 20, 22.5, 25, 30, 35, 40, 45, 50, 75, 100, 150, 200, 0.5-1.5, 0.8-1.5, 1.0-1.5, 0.5-1.4, 0.8-1.4, or 1.0-1.4 mil (1 mil=0.001 inch), for instance. In some aspects, such thickness is uniform, which can be characterized by having a contiguous area that (i) is at least 20%, 30%, 40%, or 50% of the total film/coating area, and (ii) has a standard deviation of thickness of less than about 0.06, 0.05, or 0.04 mil. A film or coating herein can be characterized as thin (e.g., <2 mil) in some aspects. A film herein is typically a cast film.

A film or coating herein can exhibit various degrees of transparency as desired. For example, a film/coating can be highly transparent (e.g., high light transmission, and/or low haze). Optical transparency as used herein can, for example, refer to a film or coating allowing at least about 10-99% light transmission, or at least about 50%, 60%, 70%, 80%, 90%, 95%, 96%, 97%, 98%, or 99% light transmission, and/or less than 30%, 25%, 20%, 15%, 10%, 5%, 2.5%, 2%, or 1% haze. High optical transparency can optionally refer to a film/coating having at least about 90% light transmittance and/or a haziness of less than 10%. Light transmittance of a film/coating herein can be measured following test ASTM D1746 (2009, Standard Test Method for Transparency of Plastic Sheeting, ASTM International, West Conshohocken, PA), for example, which is incorporated herein by reference. Haze of a film/coating herein can be measured following test ASTM D1003-13 (2013, Standard Test Method for Haze and Luminous Transmittance of Transparent Plastics, ASTM International, West Conshohocken, PA), for example, which is incorporated herein by reference.

A film or coating herein can optionally further comprise a plasticizer such as glycerol, propylene glycol, ethylene glycol, and/or polyethylene glycol. In some aspects, other film components (in addition to a composition herein) can be as disclosed in U.S. Patent. Appl. Publ. No. 2011/0151224, 2015/0191550, 20190153674, or 20210095155, or U.S. Pat. No. 9,688,035 or U.S. Pat. No. 3,345,200, all of which are incorporated herein by reference.

A film or coating, or any suitable solid composition herein (e.g., composite, fibers, fibrids), in some aspects can further comprise at least one crosslinking agent. Glucan ester derivative molecules of the present disclosure can be crosslinked (covalently) to each other and/or to at least one other component (e.g., polymer, active agent) of the composition, or to a component of a substrate if the composition is applied to the substrate. Yet, in some aspects, glucan ester derivative molecules herein are not crosslinked in any manner, but one or more other components of the composition are crosslinked. Crosslinking can (i) enhance the tensile strength of, and/or (ii) plasticize, a film or coating composition, for example. Crosslinking can link a film or coating to a substrate in some aspects. In some cases, a crosslinking agent such as a di- or poly-carboxylic acid, aldehyde, or polyphenol can be used to impart both plasticity and linking-to-substrate features. Suitable crosslinking agents for preparing a composition herein with crosslinking as above are contemplated to include phosphoryl chloride (POCl3), polyphosphate, sodium trimetaphosphate (STMP), boron-containing compounds (e.g., boric acid, diborates, tetraborates such as tetraborate decahydrate, pentaborates, polymeric compounds such as Polybor®, alkali borates), polyvalent metals (e.g., titanium-containing compounds such as titanium ammonium lactate, titanium triethanolamine, titanium acetylacetonate, or polyhydroxy complexes of titanium; zirconium-containing compounds such as zirconium lactate, zirconium carbonate, zirconium acetylacetonate, zirconium triethanolamine, zirconium diisopropylamine lactate, or polyhydroxy complexes of zirconium), glyoxal, glutaraldehyde, aldehyde, polyphenol, divinyl sulfone, epichlorohydrin, polyamide-epichlorohydrin (PAE), di- or poly-carboxylic acids (e.g., citric acid, malic acid, tartaric acid, succinic acid, glutaric acid, adipic acid), dichloro acetic acid, polyamines, 1,2,7,8-diepoxyoctane, diethylene glycol dimethyl ether (diglyme), a diglycidyl ether (e.g., diglycidyl ether itself, ethylene glycol diglycidyl ether [EGDGE], 1,4-butanediol diglycidyl ether [BDGE], polyethylene glycol diglycidyl ether [PEGDE, such as PEG2000DGE], 1,6-hexanediol diglycidyl ether, neopentyl glycol diglycidyl ether, bisphenol A diglycidyl ether [BADGE]) and triglycidyl ether (e.g., trimethylolpropane triglycidyl ether). Still other examples of suitable crosslinking agents are described in U.S. Pat. Nos. 4,462,917, 4,464,270, 4,477,360 and 4,799,550, and U.S. Patent Appl. Publ. No. 2008/0112907, which are all incorporated herein by reference. Yet, in some aspects, a crosslinking agent is not a boron-containing compound (e.g., as described above). Glucan ester derivative molecules herein can be crosslinked, such as with any crosslinker as presently disclosed, in other contexts besides a film or coating (e.g., in a dispersion, fiber, fibrid, or other composition disclosed herein).

One or more conditioning agents can be comprised in a film of coating, for example, to enhance the haptics of the film or coating. A conditioning agent can be an anionic softener such as sulphated oil, soap, sulphated alcohol, and/or oil emulsion; a cationic softener such as a quaternary ammonium compound; a nonionic softener such as a polyoxyethylene derivative, polyethylene emulsion, wax emulsion, and/or silicon softener; natural fatty acid; oil; monoglyceride; diglyceride; polyglyceride; citric acid ester; lactic acid ester; and/or sugar ester such as a sucrose ester and/or sorbitan ester.

Also disclosed are articles comprising an adhesive, film, coating, or binder comprising a glucan ester derivative herein in a dry form. Such articles (optionally, “coated articles”) comprise a substrate having at least one surface on which is disposed/deposited the coating, adhesive, film, or binder, in a substantially continuous or discontinuous manner. In some aspects, an article comprises paper, leather, wood, metal, polymer, fibrous material, masonry, drywall, plaster, and/or an architectural surface. An “architectural surface” herein is an external or internal surface of a building or other man-made structure. In some aspects, an article comprises a porous substrate such as in paper, cardboard, paperboard, corrugated board, a cellulosic substrate, a textile, or leather. Yet, in some aspects, an article can comprise a polymer such as polyamide, polyolefin, polylactic acid, polyethylene terephthalate (PET), poly (trimethylene terephthalate) (PTT), aramid, polyethylene sulfide (PES), polyphenylene sulfide (PPS), polyimide (PI), polyethylene imine (PEI), polyethylene naphthalate (PEN), polysulfone (PS), polyether ether ketone (PEEK), polyethylene, polypropylene, poly (cyclic olefins), poly (cyclohexylene dimethylene terephthalate), poly (trimethylene furandicarboxylate) (PTF), or cellophane. In some aspects, an article comprising a fibrous substrate is a fiber, yarn, fabric, fabric blend, textile, non-woven, paper, or carpet. A fibrous substrate can contain natural and/or synthetic fibers, such as cotton, cellulose, wool, silk, rayon, nylon, aramid, acetate, polyurethane urea, acrylic, jute, sisal, sea grass, coir, polyamide, polyester, polyolefin, polyacrylonitrile, polypropylene, polyaramid, or blends thereof.

A film, coating, or other composition (e.g. composite) herein can have grease/oil and/or oxygen barrier properties in some aspects. Such a composition can comprise, along with a glucan ester derivative herein, one or more components as disclosed in U.S. Patent. Appl. Publ. No. 20190153674 or 20210095155, which are each incorporated herein by reference. For example, a film, coating, or other composition herein can comprise, optionally as a binder, one or more of polyvinyl alcohol, polyvinyl acetate, partially saponified polyvinyl acetate, silanol-modified polyvinyl alcohol, butenediol vinyl alcohol co-polymer (BVOH), polyurethane, starch, corn dextrin, carboxymethyl cellulose, cellulose ethers, hydroxyethyl cellulose, hydroxypropyl cellulose, ethyl hydroxyethyl cellulose, methyl cellulose, alginates, sodium alginate, xanthan, carrageenan, casein, soy protein, guar gums, synthetic polymers, styrene butadiene latex, and/or styrene acrylate latex. A composition for preparing a film, coating, or other composition in some aspects can comprise about 50, 55, 60, 65, 70, 75, 80, 85, 90, 95, 65-85, 65-80, 70-85, or 70-80 wt % of a binder or compound such as polyvinyl alcohol (or any other of the above-referenced compounds), and about 50, 45, 40, 35, 30, 25, 20, 15, 10, 5, 2.5, 15-35, 20-35, 15-30, or 20-30 wt % glucan ester derivative as presently disclosed. In some aspects, a composition for preparing a film, coating, or other composition can comprise a ratio of binder or compound (e.g., any of the above-referenced compounds such as polyvinyl alcohol or starch) to glucan ester derivative herein of about 7:3, 7.5:2.5, 8:2, 8.5:1.5, or 9:1, based on the wt % of each of these components in the composition. In some aspects, a film, coating, or other composition does not comprise starch, while in other aspects such as an oxygen barrier, starch can be included (e.g., as disclosed in U.S. Patent Appl. Publ. No. 2011/0135912 or U.S. Pat. No. 5,621,026 or 6,692,801, which are incorporated herein by reference). Grease/oil barrier properties of a coating or film composition herein can be evaluated using a standard “KIT” type test following Technical Association of the Pulp and Paper Industry (TAPPI) Test Method T-559 cm-02 (Grease resistance test for paper and paperboard, TAPPI Press, Atlanta, GA, USA; incorporated herein by reference), for example. Good grease/oil barrier/resistance function is indicated in this test by values closer to 12 on a scale of 1 to 12. Grease/oil barrier properties, as well as water/aqueous liquid barrier properties, can be evaluated by a Cobb test, if desired. A barrier herein can have a Cobb index value of less than 20, 17.5, 15, 12.5, 10, 7.5, or 5, for example. Oxygen barrier properties of a coating or film composition herein can be evaluated by measuring the oxygen transmission rate (OTR) of the coating; OTR can be determined, for example, according to ASTM F-1927-07 (2007, Standard Test Method for Determination of Oxygen Gas Transmission Rate, Permeability and Permeance at Controlled Relative Humidity Through Barrier Materials Using a Coulometric Detector, ASTM International, West Conshohocken, PA), which is incorporated herein by reference. OTR can be determined under relative humidity conditions of about 50%-80%, 30%-55%, 35%-50%, or 30%-80%, and/or a temperature of about, or at least about, 15, 20, 25, 30, 35, 40, 45, 15-40, 15-35, 15-30, 15-25, 20-40, 20-35, 20-30, or 20-25° C., for example. Examples of substrates herein that can take advantage of a grease/oil and/or oxygen barrier coating include any of the foregoing substrates/surfaces, including a substrate comprising cellulose (e.g., paper, paperboard, cardboard, corrugated board, textile), polyethylene, polypropylene, poly lactic acid, poly(ethylene terephthalate) (e.g., MYLAR), poly (trimethylene terephthalate), polyamide, polybutylene succinate, polybutylene adipate terephthalate, polybutylene succinate adipate, poly (trimethylene furandicarboxylate), a synthetic and/or petrol-based substrate, or a bio-based substrate. Any of the foregoing film, coating, or other compositions can be in the form of a laminate or extruded product, for example, and that is optionally situated on any of the foregoing substrates.

A film, coating, or other composition (e.g., dispersion, foam, masterbatch, composite) comprising a glucan ester derivative herein can further comprise polyurethane (e.g., any as disclosed herein) in some aspects. Such a composition can comprise about 1, 5, 10, 15, 20, 35, 30, 35, 40, 45, 50, 55, 60, 5-60, 5-50, 5-45, 5-40, 5-35, 5-30, 10-60, 10-50, 10-45, 10-40, 10-35, or 10-30 wt % of a glucan ester derivative herein, for example; the balance can be comprised all or mostly of (e.g., be over 90% or 95% of) one or more polyurethanes. Such a composition can be wet (e.g., a dispersion of glucan ester derivative and polyurethane) or dry (e.g., a masterbatch, film/coating, laminate, foam, or extruded composite of glucan ester derivative and polyurethane). A polyurethane herein can be of a molecular weight that is about, or at least about, 1000, 1500, 2000, 2500, 3000, 3500, 4000, 1000-3000, 1500-3000, 1000-2500, or 1500-2500, for example. Such a composition can, in some instances, be hydrolytically aged (e.g., exposed to 45-55 or ~50° C., and/or 90-98% or ~95% relative humidity, for a period of 2-4 or 3 days). In some aspects, a polyurethane composition with a glucan ester derivative herein can be heat- and/or pressure-processable; application of heat and/or pressure for pressing, molding, extruding, or any other related processing step can be at about, or at least about, 90, 95, 100, 105, 110, 115, 120, 130, 140, 95-115, or 100-110° C., and/or at a pressure of at least about 5000, 10000, 15000, 20000, or 25000 psi, for example. Such application of heat and/or pressure can be for a time of at least about 1, 2, 3, 4, 5, 6, 7, 8, 9, 10, 15, or 30 minutes, for example. A pressed polyurethane composition in some aspects such as a film can be about, or at least about, 70%, 75%, 80%, 85%, 90%, 95%, 98%, 99%, or 100% transparent or translucent. In some aspects, any polyurethane composition presently disclosed can be made by a process that comprises providing an aqueous polyurethane dispersion, and mixing a glucan ester derivative herein with the polyurethane dispersion. The resulting aqueous composition can be used directly to make a composition (e.g., a film or coating), or it can be dried to a masterbatch that is then used to prepare a composition (e.g., by melt-processing).

A film or coating in some aspects can be in the form of an edible film or coating. Such a material can, in some aspects, comprise a glucan ester derivative herein and one or more components as described in U.S. Pat. Nos. 4,710,228, 4,543,370, 4,820,533, 4,981,707, 5,470,581, 5,997,918, 8,206,765, or 8999413, or U.S. Patent Appl. Publ. No. 2005/0214414, which are incorporated herein by reference. In some aspects, a glucan ester derivative herein replaces starch and/or starch derivatives in an edible film or coating, optionally as disclosed in any of the foregoing references. An edible film or coating can be on potato products (e.g., potato strips such as French fries), other vegetables or vegetable products (e.g., zucchini, squash, sweet potatoes, onions, okra, peppers, string beans, tomatoes, cucumbers, lettuce, cabbage, carrots, broccoli, cauliflower, Brussels sprouts, bean sprouts, onions, any fresh cut version of a vegetable), mushrooms, fruits (e.g., berries such as raspberries, strawberries, or blue berries, avocados, kiwis, kumquats, oranges, tangerines, apples, pears, bananas, grapefruit, cherries, papaya, lemons, limes, mangos, peaches, cantaloupe, any fresh cut version of a fruit), and/or nuts (peanuts, walnuts, almonds, pecans, cashews, filberts/hazel nuts, Brazil nuts, macadamias), for example. Any other food disclosed herein, as appropriate, can have an edible coating, for example. These and other food products having an edible film or coating herein can be fried or baked in some aspects, and/or the film or coating provides tenderness, moisture retention, protection from moisture, crispness, dietary fiber (in place of digestible starch), oxygen barrier, freshness, and/or anti-ripening. Anti-ripening in some aspects can be measured by the degree to which a coating lowers (e.g., by at least 25%, 50%, 75%, 80%, 85%, or 90%) the emission of a gaseous ripening hormone, such as ethylene, by a plant-based product (e.g., at 15-30, 15-25, or 20-25° C.), and/or by the degree to which plant product softening and/or sweetening is decreased by a coating. An edible coating in some aspects can be prepared by applying an aqueous dispersion or solution comprising a glucan ester derivative herein (e.g., at 5-15, 5-12, 5-10, 7.5-15, 7.5-12, or 7.5-10 wt % in water) to a food product and drying the dispersion or solution (e.g., by air drying, forced air drying, vacuum drying, and/or heating).

A coating composition in some aspects, which can be used to prepare a coating herein, can comprise any of the foregoing components/ingredients/formulations. In some aspects, a coating composition is a latex composition, such as described below.

A composition herein comprising at least one glucan ester derivative as presently disclosed can be a latex composition in some aspects. Examples of latex compositions herein include paint (e.g., primer, finishing/decorative), adhesives, films, coatings, and binders. Formulations and/or components (in addition to a composition herein) of a latex composition herein can be as described in, for example, U.S. Pat. Nos. 6,881,782, 3,440,199, 3,294,709, 5,312,863, 4,069,186, or 6297296, or U.S. Patent Appl. Publ. No. 2020/0263026, which are all incorporated herein by reference.

A glucan ester derivative as presently disclosed can be present in a latex composition in any useful amount, such as at about, or at least about, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 40%, 45%, 50%, 55%, 60%, 65%, 70%, 75%, 0.01%-75% 0.01%-5%, 5%-20%, 20%-50%, or 50%-75% based on the weight of all the dispersed solids of the latex.

A latex composition in some aspects can comprise a polymer polymerized from at least one ethylenically unsaturated monomer (e.g., monoethylenically unsaturated monomer); polyurethane; epoxy, and/or a rubber elastomer. Examples of monoethylenically unsaturated monomers herein include vinyl monomers, acrylic monomers, allylic monomers, acrylamide monomers, monocarboxylic unsaturated acids and dicarboxylic unsaturated acids.

Examples of suitable vinyl monomers of a polymer in a latex composition herein include any compound having vinyl functionality (i.e., ethylenic unsaturation) such as vinyl esters (e.g., vinyl acetate, vinyl propionate, vinyl laurate, vinyl pivalate, vinyl nonanoate, vinyl decanoate, vinyl neodecanoate, vinyl butyrates, vinyl benzoates, vinyl isopropyl acetates), vinyl aromatic hydrocarbons (e.g., styrene, methyl styrenes and similar lower alkyl styrenes, chlorostyrene, vinyl toluene, vinyl naphthalene, divinyl benzene), vinyl aliphatic hydrocarbons (e.g., vinyl chloride; vinylidene chloride; alpha olefins such as ethylene, propylene and isobutylene; conjugated dienes such as 1,3-butadiene, methyl-2-butadiene, 1,3-piperylene, 2,3-dimethyl butadiene, isoprene, cyclohexene, cyclopentadiene, and dicyclopentadiene) and vinyl alkyl ethers (e.g., methyl vinyl ether, isopropyl vinyl ether, n-butyl vinyl ether, isobutyl vinyl ether), but excluding compounds having acrylic functionality (e.g., acrylic acid, methacrylic acid, esters of such acids, acrylonitrile, acrylamides). In some aspects, a latex composition herein comprises a vinyl acetate-ethylene copolymer, carboxylated vinyl acetate-ethylene copolymer, and/or or polyvinyl acetate.

Examples of suitable acrylic monomers of a polymer in a latex composition herein include alkyl acrylates, alkyl methacrylates, acrylate acids, methacrylate acids, aromatic derivatives of acrylic and methacrylic acid, acrylamides, and acrylonitrile. Typically, alkyl acrylate and methacrylic monomers (also referred to as alkyl esters of acrylic or methacrylic acid) have an alkyl ester portion containing from 1 to about 18 carbon atoms per molecule, or from 1 to about 8 carbon atoms per molecule. Suitable acrylic monomers include, for example, methyl acrylate and methacrylate, ethyl acrylate and methacrylate, butyl acrylate and methacrylate, propyl acrylate and methacrylate, 2-ethyl hexyl acrylate and methacrylate, cyclohexyl acrylate and methacrylate, decyl acrylate and methacrylate, isodecyl acrylate and methacrylate, benzyl acrylate and methacrylate, isobornyl acrylate and methacrylate, neopentyl acrylate and methacrylate, and 1-adamantyl methacrylate. If acid functionality is desired, acids such as acrylic acid or methacrylic acid can also be used.

A latex composition in some aspects comprises a polyurethane polymer. Examples of suitable polyurethane polymers are those comprising polysaccharides as disclosed in U.S. Patent Appl. Publ. No. 2019/0225737, which is incorporated herein by reference. A latex comprising a polyurethane can be prepared, for example, as disclosed in U.S. Patent Appl. Publ. No. 2016/0347978, which is incorporated herein by reference, and/or comprise the reaction product of one or more polyisocyanates with one or more polyols. Useful polyols include polycarbonate polyols, polyester polyols and polyether polyols, for example. Polycarbonate polyurethane herein can be formed as the reaction product of a polyol such as 1,3-propanediol, 1,4-butanediol, 1,6-hexanediol, diethylene glycol, or tetraethylene glycol, with a diaryl carbonate such as diphenyl carbonate or phosgene. At least one polyisocyanate herein can be an aliphatic polyisocyanate, aromatic polyisocyanate, or polyisocyanate that has both aromatic and aliphatic groups. Examples of polyisocyanates include 1,6-hexamethylene diisocyanate, isophorone diisocyanate, 2,4-toluene diisocyanate, 2,6-toluene diisocyanate, mixtures of 2,4- and 2,6-toluene diisocyanate, bis(4-isocyanatocyclohexyl) methane, 1,3-bis(1-isocyanato-1-methylethyl)benzene, bis(4-isocyanatophenyl) methane, 2,4′-diphenylmethane diisocyanate, 2,2′-diphenylmethane diisocyanate, 2,4-diisocyanatotoluene, bis(3-isocyanatophenyl) methane, 1,4-diisocyanatobenzene, 1,3-diisocyanato-o-xylene, 1,3-diisocyanato-p-xylene, 1,3-diisocyanato-m-xylene, 2,4-diisocyanato-1-chlorobenzene, 2,4-diisocyanato-1-nitrobenzene, 2,5-diisocyanato-1-nitrobenzene, m-phenylene diisocyanate, hexahydrotoluene diisocyanate, 1,5-naphthalene diisocyanate, 1-methoxy-2,4-phenylene diisocyanate, 4,4′-biphenylmethane diisocyanate, 4,4′-biphenylene diisocyanate, 3,3′-dimethyl-4,4′-diphenylmethane, diisocyanate, 3,3′-4,4′-diphenylmethane diisocyanate, and 3,3′-dimethyldiphenylmethane-4,4′-diisocyanate. Also useful herein are polyisocyanate homopolymers comprising allophanate, biuret, isocyanurate, iminooxadiazinedione, or carbodiimide groups, for example. A polyol herein can be any polyol comprising two or more hydroxyl groups, for example, a C2 to C12 alkane diol, ethylene glycol, 1,2-propylene glycol, 1,3-propylene glycol, isomers of butane diol, pentane diol, hexane diol, heptane diol, octane diol, nonane diol, decane diol, undecane diol, dodecane diol, 2-methyl-1,3-propane diol, 2,2-dimethyl-1,3-propane diol (neopentyl glycol), 1,4-bis(hydroxymethyl)cyclohexane, 1,2,3-propane triol (glycerol), 2-hydroxymethyl-2-methyl-1,3-propanol (trimethylolethane), 2-ethyl-2-hydroxymethyl-1,3-propanediol (trimethylolpropane), 2,2-bis(hydroxymethyl)-1,3-propane diol (pentaerythritol); 1,4,6-octanetriol; chloropentanediol; glycerol monoalkyl ether; glycerol monoethyl ether; diethylene glycol; 1,3,6-hexanetriol; 2-methylpropanediol; 2,2,4-trimethyl-1,3-pentanediol, cyclohexanedimethanol, polymeric polyols, for example, polyether polyols or polyester polyols. In some aspects, a polyol herein can be poly (oxytetramethylene) glycol, polyethylene glycol, or poly 1,3-propane diol. A polyol in some aspects can be polyester polyol, such as one produced by transesterification of aliphatic diacids with aliphatic diols. Suitable aliphatic diacids include, for example, C3 to C10 diacids, malonic acid, succinic acid, glutaric acid, adipic acid, pimelic acid, suberic acid, azelic acid, sebacic acid. In some aspects, aromatic and/or unsaturated diacids can be used to form a polyester polyol.

A latex composition in some aspects comprises an epoxy polymer/resin (polyepoxide), such as bisphenol A epoxy resin, bisphenol F epoxy resin, Novolac epoxy resin, aliphatic epoxy resin, or glycidylamine epoxy resin.

A latex composition in some aspects comprises a rubber elastomer. In some aspects, a rubber elastomer can include one or more diene-based sulfur-vulcanizable elastomers having a glass transition temperature (Tg) below −30° C., as determined, for example, by dynamic mechanical analysis. In further examples, a rubber elastomer herein includes natural rubber, synthetic polyisoprene, polybutadiene rubber, styrene/butadiene copolymer rubber, ethylene propylene diene monomer rubber, hydrogenated nitrile butadiene rubber, neoprene, styrene/isoprene/butadiene terpolymer rubber, butadiene/acrylonitrile rubber, polyisoprene rubber, isoprene/butadiene copolymer rubber, nitrile rubber, ethylene-acrylic rubber, butyl and halobutyl rubber, chlorosulfonated polyethylene, fluoroelastomer, hydrocarbon rubber, polybutadiene, or silicone rubber.

The liquid component of a latex composition herein can be water or an aqueous solution. An aqueous solution of a latex in some aspects can comprise an organic solvent that is either miscible or immiscible with water. Suitable organic solvents herein include acetone, methyl ethyl ketone, butyl acetate, tetrahydrofuran, methanol, ethanol, isopropanol, diethyl ether, glycerol ethers, hexane, toluene, dimethyl acetamide, dimethylformamide, and dimethyl sulfoxide.

A latex composition herein can further comprise one or more additives in some aspects. Examples of additives herein include dispersants, rheological aids, antifoams, foaming agents, adhesion promoters, flame retardants, bactericides, fungicides, preservatives, optical brighteners, fillers, anti-settling agents, coalescing agents, humectants, buffers, pigments/colorants (e.g., metallic oxides, synthetic organic pigments, carbon black), viscosity modifiers, antifreeze, surfactants, binders, crosslinking agents, anticorrosion agents, hardeners, pH regulators, salts, thickeners, plasticizers, stabilizers, extenders, and matting agents. Examples of pigments herein include titanium dioxide (TiO₂), calcium carbonate, diatomaceous earth, mica, hydrated aluminum oxide, barium sulfate, calcium silicate, clay, silica, talc, zinc oxide, aluminum silicate, nepheline syenite, and mixtures thereof. In some aspects, a latex composition is essentially free from (e.g., less than 1, 0.5, 0.1, or 0.01 wt % of component) starch, starch derivative (e.g., hydroxyalkyl starch), cellulose, and/or cellulose derivative (e.g., carboxymethyl cellulose).

A latex composition in the form of a paint or other coloring agent herein can have a pigment volume concentration (PVC) of about 3% to about 80% in some aspects. As examples, a flat paint can have a PVC in the range of about 55-80%, a primer or undercoat can have a PVC in the range of about 30-50%, and/or a gloss colored paint can have a PVC in the range of about 3-20%. A paint or other coloring agent in some aspects can have a PVC of about 55%, 60%, 65%, 70%, 75%, 80%, 55-80%, 55-75%, 55-70%, 60-80%, 60-75%, 60-70%, 63-67%, 64-66%, 65-80%, 65-75%, or 65-70%. A PVC value herein can be that of a particular pigment (or mix of pigments) such as those disclosed above (e.g., titanium dioxide), for instance. A composition of the present disclosure is believed to provide one or more physical properties to a latex composition (e.g., for use as a paint or other coloring agent): opacity, less pigment needed, increased hardness, reduced tackiness, decreased gloss (i.e., providing a matte effect), increased shear strength, better abrasion resistance, improved dry time, improved fade resistance, lower blistering, and/or improved hand (a less tacky feel), for example, as compared to a latex composition that only differs by not comprising the disclosed composition.

A latex composition herein can be applied to the substrate of an article (above) using any method known in the art. Typically, after application of the latex composition, at least a portion of the aqueous solution is removed, for example by drying, to provide an adhesive, film, coating, or binder comprising the latex composition in a dry or semi-dry form. Suitable application methods include air knife coating, rod coating, bar coating, wire bar coating, spray coating, brush coating, cast coating, flexible blade coating, gravure coating, jet applicator coating, short dwell coating, slide hopper coating, curtain coating, flexographic coating, size-press coating, reverse roll coating, and transfer roll coating. A latex composition can be applied on at least a portion of a substrate, and can be in one or more coats/applications, for example.

Some aspects herein are drawn to a pigment-comprising composition. A pigment-comprising composition can be in a liquid form (e.g., an aqueous or non-aqueous composition herein) or solid form (e.g., a dry composition herein). Examples of a pigment-comprising composition herein include any of such compositions disclosed elsewhere herein (e.g., paint, primer, stain), ink, dye (e.g., food-coloring dye, fabric-coloring dye), resin, sunscreen, and cosmetics (e.g., mascara, blush, nail varnish/polish, lipstick, gloss, eyeliner, foundation, eye shadow, skin decoration composition). A pigment in a pigment-comprising composition can be any pigment herein, for example. Examples of a pigment for these and/or other aspects herein include oxides of titanium (e.g., titanium dioxide), zinc, iron, zirconium, cerium, and chromium; manganese violet; ultramarine blue; chromium hydrate; Prussian Blue; zinc sulfide; nitroso, nitro, azo, xanthene, quinoline, anthraquinone and/or phthalocyanine compounds; metal complex compounds; and isoindolinone, isoindoline, quinacridone, perinone, perylene, diketopyrrolopyrrole, thioindigo, dioxazine, triphenylmethane and/or quinophthalone compounds. Further pigment examples useful herein are disclosed in U.S. Patent. Appl. Publ. No. 2006/0085924, which is incorporated herein by reference.

A composition herein comprising at least one glucan ester derivative as presently disclosed can be in the form of a composite (e.g., rubber composite or polyurethane composite) such as disclosed in U.S. Patent Appl. Publ. Nos. 2019/0225737, 2017/0362345, or 2020/0181370, all of which are incorporated herein by reference. It can optionally be stated that a composite as presently disclosed comprises at least one polymer in addition to a glucan ester derivative of the disclosure. One or more of the above components (e.g., a rubber or polyurethane) of a latex composition can optionally be an additional polymer in such a composite. An additional polymer of a composite herein can be rubber, polyurethane, thermoplastic polymer, polyethylene, polypropylene, ethylene copolymer, polyvinyl butyrate, polylactic acid, polyvinyl alcohol, polyamide, polyether thermoplastic elastomer, polyester, polyether ester, ethylene vinyl alcohol copolymer, starch, cellulose, or any suitable polymer as disclosed above for latex components.

Rubber in some aspects can be one or more of natural rubber, synthetic rubber, polyisoprene, polybutadiene, styrene-butadiene copolymer, styrene-isoprene copolymer, butadiene-isoprene copolymer, styrene-butadiene-isoprene terpolymer, ethylene propylene diene monomer rubber, hydrogenated nitrile butadiene rubber, silicone rubber, or neoprene, for instance. Examples of composites herein comprising rubber include tires (e.g., auto/bicycle; pneumatic tire; including tire treads and/or sidewalls), belts (e.g., conveyor belts, power transmission belts), hoses, gaskets, footwear (e.g., shoes, sneakers, boots; soles, cushioning, and/or aesthetic features), coatings, films, and adhesives. Rubber composites herein typically are vulcanized. It is contemplated that, in some aspects, inclusion of a composition herein in a composite comprising rubber can provide advantages such as lower cost, lower density, lower energy consumption during processing, and/or better or equal performance as compared to use of an incumbent filler such as carbon black or silica (e.g., increased wet traction, reduced rolling resistance, lighter weight, and/or mechanical strength); such performance enhancements can be with tires in some aspects.

In some aspects, a composition herein replaces about, or at least about, 25, 30, 35, 40, 45, 50, 55, 60, 65, 70, 75, 80, 85, 90, 95, or 100 wt % of an incumbent filler (e.g., carbon black, or silica) that is typically used in a rubber composite such as a tire. It is noted that rubber composite tires currently on the market (that do not comprise a composition herein) typically comprise up to about 30 wt % of an incumbent filler such as carbon black. Thus, a rubber composite herein such as a tire can comprise about, or at least about, 5, 10, 15, 20, 25, or 30 wt % a composition as presently disclosed, for example. A rubber composition herein can have a low minimum elastic torque (ML) (e.g., less than, or about, 0.10, 0.08, 0.06, 0.04, 0.03, or 0.02 dNm [deciNewton-meter]) in some aspects, and so a method of mixing a rubber composition during its preparation is disclosed.

A composition comprising at least one glucan ester derivative as presently disclosed can be a paper/packaging composition or cellulose fiber-containing composition. Examples of such compositions can be any type of paper/packaging or cellulose fiber-containing composition disclosed herein, such as paper (e.g., writing paper, office paper, copying paper, crafting paper), cardboard, paperboard, corrugated board, tissue paper, napkin/paper towel, wipe, or a non-woven fabric. Formulations and/or components (in addition to a glucan ester derivative herein) of a paper/packaging composition or cellulose fiber-containing composition herein, and well as forms of these compositions, can be as described in, for example, U.S. Patent Appl. Publ. Nos. 2018/0119357, 2019/0330802, 2020/0062929, 2020/0308371, or 2020/0370216, which are all incorporated herein by reference. In some aspects, a glucan ester derivative functions as a strengthening aid in paper or other cellulose fiber-containing composition. It is contemplated that the ability of a glucan ester derivative to flocculate fiber and/or other insoluble material in a papermaking process (e.g., pulp flocculation) is a means in which a glucan ester derivative herein can be incorporated into paper or other product that involves flocculation in its production. In some aspects, though, a glucan ester derivative herein can be added as a component in any of the forgoing compositions in a manner independent from its possible addition as a flocculation aid.

Some aspects of the present disclosure regard a flocculation or dewatering method that comprises: (a) mixing at least one glucan ester derivative herein into an aqueous composition that comprises suspended solids/particles, whereby at least a portion of the suspended solids/particles becomes flocculated; and (b) optionally, separating the flocculated solids/particles of (a) from the aqueous composition. A glucan ester derivative in some aspects can therefore be characterized as a flocculation agent, dewatering agent, clarification agent, and/or de-clouding agent, for instance. The flocculated particles of a treated composition typically settle (floc), or at least become more amenable to separation procedures (e.g., filtration). While a soluble glucan ester derivative can be used in the flocculation method, an insoluble glucan ester derivative can be used in some aspects. Typically, a glucan ester derivative herein for flocculation applications is (i) biodegradable and/or (ii) not crosslinked.

One, two, three, or more different types of glucan ester derivative herein can be used in a flocculation method, for instance. In some aspects, a glucan ester derivative is the only flocculation agent employed, whereas in other aspects, a glucan ester derivative is used in addition to another type of flocculation agent (e.g., a commercial incumbent flocculating agent such as acrylamide). In these latter aspects, a glucan ester derivative can constitute about, or at least about 30, 40, 50, 60, 70, 80, or 90 wt %, for example, of all the flocculation agents added to an aqueous composition.

The amount of a glucan ester derivative that is mixed in step (a) in an aqueous composition that comprises suspended solids/particles can be about, or at least about, 2, 4, 6, 8, 10, 12, 14, 2-14, 2-12, 2-10, 2-8, 4-14, 4-12, 4-10, 4-8, 6-14, 6-12, 6-10, 6-8, 8-14, 8-12, or 8-10 g per kg (dry solids basis) of suspended solids, for example. It would be understood that an aqueous-soluble glucan ester derivative is typically dissolved in the aqueous composition after mixing step (a). Mixing can be performed by any standard means.

The temperature and pH of an aqueous composition with suspended solids that is treated with a glucan ester derivative can be any temperature and pH as disclosed herein for an aqueous composition. In some aspects, the pH can be about 4, 5, 6, 7, 8, 9, 10, 4-10, 5-9, or 6-8, and/or the temperature can be about 1-80, 1-70, 1-60, 1-50, 1-40, 1-30, 5-80, 5-70, 5-60, 5-50, 5-40, 5-30, 15-80, 15-70, 15-60, 15-50, 15-40, or 15-30° C. Upon adding and mixing a glucan ester derivative with an aqueous composition, settling of suspended solids can be allowed to commence for about, or at least about, 0.5, 1, 2, 3, 4, 5, 6, 9, 12, 18, 24, 30, 36, 42, or 48 hours, for example.

In some aspects, the percentage of the initially suspended solids that settle (i.e., are no longer suspended) following treatment with a glucan ester derivative is about, or at least about, 30, 40, 50, 60, 70, 80, 90, 95, 96, 97, 98, 99, or 100 wt %. Typically, a flocculation agent herein allows settled particles to occupy less space. For example, the total volume of settled particles following treatment of an aqueous composition (initially having suspended particles) with a glucan ester derivative herein can be about, or less than about, 90%, 80%, 70%, 60% or 50% of the total volume of settled particles that settle in an aqueous composition without the aid of a flocculation agent (where all other conditions of each system are the same). Any suitable method can be used to determine settling volume, such as the method described in the below Examples.

In some aspects, the turbidity (i.e., the quality of a liquid being cloudy, opaque, and/or thick with suspended matter), color and/or opacity of an aqueous composition having suspended solids/particles can be reduced by about, or at least about, 5%, 10%, 15%, 20%, 25%, 30%, 35%, 40%, 45%, 50%, 55%, 60%, 65%, 70%, or 75% when treated with a glucan ester derivative herein. Turbidity can be measured in nephelometric turbidity units (NTU), for example. Any suitable method can be used to measure turbidity, such as the methodology disclosed in Progress in Filtration and Separation (Edition: 1, Chapter 16. Turbidity: Measurement of Filtrate and Supernatant Quality?, Publisher: Academic Press, Editors: E.S. Tarleton, July 2015), which is incorporated herein by reference, or as described in the below Examples. Any suitable method can be used to measure the color of a liquid herein, such as spectral colorimetry or photoelectric colorimetry, for example.

In some aspects, the filterability of an aqueous composition having suspended solids/particles can be enhanced/increased when treated with a glucan ester derivative herein. The filterability of a liquid composition can be measured using any suitable method, such as by measuring capillary suction time. In some aspects, the capillary suction time (e.g., as measured in seconds) of an aqueous composition having suspended solids/particles can be decreased by about, or at least about, 10%, 20%, 30%, 40%, 50%, 60%, 70%, 80%, or 85% when treated with a glucan ester derivative herein. Any suitable method can be used to measure capillary suction time of a liquid.

Suspended particles that can be subjected to flocculation herein typically are colloidal particles (i.e., undissolved particles [solids] that are stably suspended). Thus, an aqueous composition that can be subjected to a flocculation method herein can be a colloid, for example. An aqueous composition comprising suspended solids/particles that can be treated with a flocculation agent as presently disclosed can be waste water (e.g., municipal, industrial, agricultural), sewage/sewage water, sludge (e.g., activated sludge), water from a body of water (e.g., river/stream, canal, moat, pond, marsh, lake, ocean), pool water, cooling water, water containing sediment (e.g. clay sediment) and/or soil, water to be processed for drinking, or water containing fiber and/or filler such as present in a paper-making process (e.g., pulp flocculation), for example. Examples of industrial waste water are from paper mill or drilling/mining operations. In some aspects, suspended solids can comprise microbial cells (live and/or dead) such as bacteria, yeast, and/or algae. It is contemplated that flocculation herein can be applied to an aqueous composition present during a food- or beverage-making process such as brewing (e.g., wort after its fermentation), cheese curd formation or soy curd (tofu) production. Systems/operations that can incorporate the disclosed flocculation method include waste water/sewage/sludge treatment, paper making, water purification, soil conditioning, and/or mining/drilling/downhole operations, for example, or any other system/operation that employs flocculation.

A flocculation method herein optionally further comprises a step of separating flocculated solids/particles of from the treated aqueous composition. Such a step can comprise settling/sedimentation, filtration, centrifugation, and/or decanting, for example.

The present disclosure also concerns a method of producing a solid composition comprising at least one glucan ester derivative herein. Such a method can comprise at least (a) providing a non-caustic (e.g., pH 6-8 or 6-9) aqueous composition (e.g., solution or dispersion) comprising at least one glucan ester derivative as presently disclosed, (b) putting the aqueous composition into a desired form (e.g., fiber, fibrid, film/coating, composite, extrusion), and (c) removing the liquid/solvent from the aqueous composition of step (b) to produce a solid composition comprising the glucan ester derivative. In some aspects, the ester derivative is of a glucan herein that, as a non-derivative, is insoluble under non-caustic aqueous conditions (e.g., alpha-1,3-glucan of DP>8 or >9).

In some aspects in which the non-caustic aqueous composition is a solution and the ester derivative is of a glucan herein that, as a non-derivative, is insoluble under non-caustic aqueous conditions, liquid/solvent can be removed by raising the pH of the solution to over about 10, 10.5, 11, 11.5, or 12, thereby causing the dissolved glucan ester to precipitate out of solution. The pH of the solution can be raised, for example, by adding/mixing a base (e.g., a metal hydroxide such as NaOH) to the solution. The precipitated glucan ester derivative—which is in a desired form/shape from step (b)—can optionally be washed with an organic liquid such as an alcohol (e.g., methanol, ethanol, isopropanol), and/or dried, for example. The concentration of a glucan ester derivative in a solution provided in step (a) can be as disclosed elsewhere herein, such as about, or at least about, 10, 12, 14, 16, 18, 20, 25, 30, 10-30, 10-25, 10-20, 16-30, 16-25, or 16-20 wt %.

The present disclosure also regards a solid composition as produced by the foregoing process. Such a composition can be a fiber, fibrid, film/coating, composite, or extrusion, for example.

Non-limiting examples of compositions and methods disclosed herein include:
1. A composition comprising an ester derivative of a glucan (glucan ester derivative), wherein the glucan has a degree of substitution (DoS) up to about 3.0 with at least two organic groups that are individually ester-linked to the glucan, wherein (i) at least one of the organic groups is a cationic organic group, and (ii) at least one of the organic groups is a hydrophobic organic group.
2. The composition of embodiment 1, wherein the glucan is an alpha-glucan.
3. The composition of embodiment 2, wherein at least about 50% of the glycosidic linkages of the alpha-glucan are alpha-1,3 linkages.
4. The composition of embodiment 2, wherein at least about 50% of the glycosidic linkages of the alpha-glucan are alpha-1,6 linkages, optionally wherein the alpha-glucan comprises at least 1% alpha-1,2 and/or alpha-1,3 branches.
5. The composition of embodiment 1, wherein the glucan is a beta-glucan (e.g., wherein at least about 50% of the glycosidic linkages of the beta-glucan are beta-1,3 linkages or beta-1,4 linkages).
6. The composition of embodiment 1, 2, 3, 4, or 5, wherein the glucan has a weight-average degree of polymerization (DPw) of at least 6.
7. The composition of embodiment 1, 2, 3, 4, 5, or 6, wherein the DoS with the at least two organic groups is at least about 0.005.
8. The composition of embodiment 1, 2, 3, 4, 5, 6, or 7, wherein the DoS with the at least two organic groups is about 0.005 to about 1.5.
9. The composition of embodiment 1, 2, 3, 4, 5, 6, 7, or 8, wherein the DoS with the cationic organic group is about 0.005 to about 1.5.
10. The composition of embodiment 1, 2, 3, 4, 5, 6, 7, 8, or 9, wherein the DoS with the cationic organic group is about 0.005 to about 1.0.
11. The composition of embodiment 1, 2, 3, 4, 5, 6, 7, 8, 9, or 10, wherein the DoS with the cationic organic group is about 0.005 to about 0.5.
12. The composition of embodiment 1, 2, 3, 4, 5, 6, 7, 8, 9, 10, or 11, wherein the DoS with the cationic organic group is about 0.005 to about 0.1.
13. The composition of embodiment 1, 2, 3, 4, 5, 6, 7, 8, 9, 10, 11, or 12, wherein the DoS with the cationic organic group is about 0.04 to about 0.1.
14. The composition of embodiment 1, 2, 3, 4, 5, 6, 7, 8, 9, 10, 11, 12 or 13, wherein the DoS with the hydrophobic organic group is about 0.005 to about 1.5.
15. The composition of embodiment 1, 2, 3, 4, 5, 6, 7, 8, 9, 10, 11, 12, 13, or 14, wherein the DoS with the hydrophobic organic group is about 0.005 to about 1.0.
16. The composition of embodiment 1, 2, 3, 4, 5, 6, 7, 8, 9, 10, 11, 12, 13, 14, or 15, wherein the DoS with the hydrophobic organic group is about 0.005 to about 0.5.
17. The composition of embodiment 1, 2, 3, 4, 5, 6, 7, 8, 9, 10, 11, 12, 13, 14, 15, or 16, wherein the DoS with the hydrophobic organic group is about 0.4 to about 1.0.
18. The composition of embodiment 1, 2, 3, 4, 5, 6, 7, 8, 9, 10, 11, 12, 13, 14, 15, 16, or 17, wherein the cationic organic group comprises the structure:

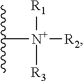

and each of R₁, R₂ and R₃ is independently a group comprising at least one carbon atom (e.g., each of R₁, R₂ and R₃ is CH₃).
19. The composition of embodiment 1, 2, 3, 4, 5, 6, 7, 8, 9, 10, 11, 12, 13, 14, 15, 16, 17, or 18, wherein the cationic organic group comprises the structure:

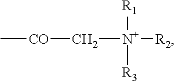

and each of R₁, R₂ and R₃ is independently a group comprising at least one carbon atom (e.g., each of R₁, R₂ and R₃ is CH₃).
20. The composition of embodiment 1, 2, 3, 4, 5, 6, 7, 8, 9, 10, 11, 12, 13, 14, 15, 16, 17, 18, or 19, wherein the hydrophobic organic group comprises a C₂ to C₂₆ acyl group (e.g., a C₆ to C₁₈ acyl group, a C₈ to C₁₆ acyl group, a C₁₀ to C₁₄ acyl group, or a C₁₂ acyl group).
21. The composition of embodiment 1, 2, 3, 4, 5, 6, 7, 8, 9, 10, 11, 12, 13, 14, 15, 16, 17, 18, 19, or 20, wherein the hydrophobic organic group comprises an aryl group (e.g., a benzoyl group or a substituted benzoyl group).
22. The composition of embodiment 1, 2, 3, 4, 5, 6, 7, 8, 9, 10, 11, 12, 13, 14, 15, 16, 17, 18, 19, 20, or 21, wherein the ester derivative of the glucan has a biodegradability as determined by a carbon dioxide evolution test method of at least 10% after 15 days.
23. The composition of embodiment 1, 2, 3, 4, 5, 6, 7, 8, 9, 10, 11, 12, 13, 14, 15, 16, 17, 18, 19, 20, 21, or 22, wherein the composition is an aqueous composition.
24. The composition of embodiment 1, 2, 3, 4, 5, 6, 7, 8, 9, 10, 11, 12, 13, 14, 15, 16, 17, 18, 19, 20, 21, 22, or 23, wherein the composition is a household care product, personal care product, industrial product, ingestible product (e.g., food product), or pharmaceutical product.
25. The composition of embodiment 1, 2, 3, 4, 5, 6, 7, 8, 9, 10, 11, 12, 13, 14, 15, 16, 17, 18, 19, 20, 21, 22, 23, or 24, further comprising at least one surfactant.
26. The composition of embodiment 1, 2, 3, 4, 5, 6, 7, 8, 9, 10, 11, 12, 13, 14, 15, 16, 17, 18, 19, 20, 21, 22, 23, 24, or 25, further comprising at least one enzyme.
27. The composition of embodiment 26, wherein the enzyme is a cellulase, protease, lipase, amylase, lipase, or nuclease.
28. The composition of embodiment 1, 2, 3, 4, 5, 6, 7, 8, 9, 10, 11, 12, 13, 14, 15, 16, 17, 18, 19, 20, 21, 22, 23, 24, 25, 26, or 27, further comprising at least one of a complexing agent, soil release polymer, surfactancy-boosting polymer, bleaching agent, bleach activator, bleaching catalyst, fabric conditioner, clay, foam booster, suds suppressor, anti-corrosion agent, soil-suspending agent, anti-soil re-deposition agent, dye, bactericide, tarnish inhibitor, optical brightener, perfume, saturated or unsaturated fatty acid, dye transfer-inhibiting agent, chelating agent, hueing dye, visual signaling ingredient, anti-foam, structurant, thickener, anti-caking agent, starch, sand, or gelling agent.
29. The composition of embodiment 1, 2, 3, 4, 5, 6, 7, 8, 9, 10, 11, 12, 13, 14, 15, 16, 17, 18, 19, 20, 21, 22, 23, 24, 25, 26, 27, or 28, wherein the composition is in the form of, or comprised in, a liquid, gel, powder, hydrocolloid, granule, tablet, bead or pastille, single-compartment sachet, multi-compartment sachet, single-compartment pouch, or multi-compartment pouch.
30. The composition of embodiment 1, 2, 3, 4, 5, 6, 7, 8, 9, 10, 11, 12, 13, 14, 15, 16, 17, 18, 19, 20, 21, 22, 23, 24, 25, 26, 27, 28, or 29, wherein the composition is a: (a) flocculation agent, (b) viscosity modifier, (c) a latex composition, (d) a pigment-comprising composition, (e) a film or coating, (f) a fiber or fibrid, (g) a cosmetic product (e.g., hair styling product), (h) a detergent composition, (i) an adhesive composition, (j) paper, or (k) any composition/product as disclosed herein.
31. A method of producing an ester derivative of a glucan (glucan ester derivative), the method comprising: (a) contacting a glucan with at least two esterification agents, wherein at least one of the esterification agents comprises a cationic organic group, wherein at least one of the esterification agents comprises a hydrophobic organic group (optionally wherein [i] the contacting is performed in one reaction and the at least two esterification agents are provided in the reaction simultaneously, or the at least two esterification agents are provided sequentially in the reaction, or [ii] the contacting is performed in at least two separate reactions in which at least one of the reactions uses at least one esterification agent with the cationic group, and at least one of the reactions uses at least one esterification agent with the hydrophobic group), wherein at least one cationic organic group and at least one hydrophobic organic group are esterified to the glucan, thereby producing an ester derivative of the glucan (e.g., according to embodiment 1, 2, 3, 4, 5, 6, 7, 8, 9, 10, 11, 12, 13, 14, 15, 16, 17, 18, 19, 20, 21, or 22), wherein the ester derivative of the glucan has a degree of substitution (DoS) up to about 3.0 with the cationic organic group and the hydrophobic organic group, and (b) optionally, isolating the ester derivative of the glucan produced in step (a).
32. A method of styling hair, the method comprising at least steps (a) and (b), or steps (c) and (d), as follows: (a) contacting (e.g., coating) hair with a glucan ester derivative according to (or a composition according to) embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, thereby providing treated hair (or coated hair), and (b) putting the treated hair (or the coated hair) into a desired form (e.g., straightening, curling, or putting the hair into any other form that is different from the form the hair was in as it existed before step [a] or [b]); or (c) putting hair into a desired form (e.g., straightening, curling, or putting the hair into any other form that is different from the form the hair was in as it existed before step [c]), and (d) contacting (e.g., coating) the hair of step (c) with a glucan ester derivative according to (or a composition according to) embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, thereby providing treated hair (or coated hair); and (e) optionally, removing solvent, if present, that was used to deliver the glucan ester derivative to the hair in step (a) or (d).
33. A composition as recited in embodiment 1, 6, 7, 8, 9, 10, 11, 12, 13, 14, 15, 16, 17, 18, 19, 20, 21, 22, 23, 24, 25, 26, 27, 28, 29, or 30, or a method as recited in embodiment 31 or 32, but wherein “soy polysaccharide” is recited in place of “glucan”.
34. A method of producing a solid composition comprising at least one glucan ester derivative (such as from embodiment 1, 2, 3, 4, 5, 6, 7, 8, 9, 10, 11, 12, 13, 14, 15, 16, 17, 18, 19, 20, 21, or 22), wherein the method comprises at least (a) providing a non-caustic (e.g., pH 6-8 or 6-9) aqueous composition (e.g., solution or dispersion) comprising at least one glucan ester derivative as presently disclosed, (b) putting the aqueous composition into a desired form (e.g., fiber, fibrid, film/coating, composite, extrusion), and (c) removing the liquid/solvent of the aqueous composition of step (b) (e.g., by drying, and/or if a solution is used, by raising the pH of the solution to over about 10 to cause the dissolved glucan ester to precipitate out of solution) to produce a solid composition comprising the glucan ester derivative, and (d) optionally washing and/or drying the solid composition produced in step (c)
35. A composition as recited in embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 33, or a method as recited in embodiment 32 or 34, but wherein the cationic organic ester group is replaced with an anionic organic group, optionally wherein the anionic organic group is ether-linked (e.g., a carboxyalkyl such as carboxymethyl) or ester-linked (e.g., a cyclic anhydride derivative group such as succinate) to the glucan.
36. A composition as recited in embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 33, or a method as recited in embodiment 32 or 34, but wherein the hydrophobic organic ester group is replaced with an anionic organic group, optionally wherein the anionic organic group is ether-linked (e.g., a carboxyalkyl such as carboxymethyl) or ester-linked (e.g., a cyclic anhydride derivative group such as succinate) to the glucan.

Non-limiting examples of compositions and methods disclosed herein include:
1b. A composition comprising an ester derivative of a glucan, wherein the glucan has a degree of substitution (DoS) up to about 3.0 with at least one cationic organic group (cationic acyl group) that is ester-linked to the glucan.
2b. The composition of embodiment 1b, wherein the glucan is an alpha-glucan.
3b. The composition of embodiment 2b, wherein at least about 50% of the glycosidic linkages of the alpha-glucan are alpha-1,3 linkages.
4b. The composition of embodiment 2b, wherein at least about 50% of the glycosidic linkages of the alpha-glucan are alpha-1,6 linkages, optionally wherein the alpha-glucan comprises at least 1% alpha-1,2 and/or alpha-1,3 branches.
5b. The composition of embodiment 1b, wherein the glucan is a beta-glucan.
6b. The composition of embodiment 5b, wherein at least about 50% of the glycosidic linkages of the beta-glucan are beta-1,3 linkages or beta-1,4 linkages.
7b. The composition of embodiment 1b, 2b, 3b, 4b, 5b, or 6b, wherein the glucan has a weight-average degree of polymerization (DPw) of at least 6.
8b. The composition of embodiment 1b, 2b, 3b, 4b, 5b, 6b, or 7b, wherein the DoS with the cationic organic group is at least about 0.005.
9b. The composition of embodiment 1b, 2b, 3b, 4b, 5b, 6b, or 7b, wherein the DoS with the cationic organic group is at least about 0.3.
10b. The composition of embodiment 1b, 2b, 3b, 4b, 5b, 6b, or 7b, wherein the DoS with the cationic organic group is about 0.3 to about 2.0.
11b. The composition of embodiment 1b, 2b, 3b, 4b, 5b, 6b, 7b, 8b, 9b, or 10b, wherein the cationic organic group comprises the structure:

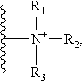

and each of R₁, R₂ and R₃ is independently a group comprising at least one carbon atom.
12b. The composition of embodiment 11b, wherein each of R₁, R₂ and R₃ is CH₃.
13b. The composition of embodiment 11b, wherein the cationic organic group comprises the structure:

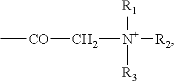

and each of R₁, R₂ and R₃ is independently a group comprising at least one carbon atom.
14b. The composition of embodiment 13b, wherein each of R₁, R₂ and R₃ is CH₃.
15b. The composition of embodiment 1b, 2b, 3b, 4b, 5b, 6b, 7b, 8b, 9b, 10b, 11b, 12b, 13b, or 14b, wherein the ester derivative of the glucan has a biodegradability as determined by a carbon dioxide evolution test method of at least 10% after 15 days.
16b. The composition of embodiment 1b, 2b, 3b, 4b, 5b, 6b, 7b, 8b, 9b, 10b, 11b, 12b, 13b, 14b, or 15b, wherein the composition is an aqueous composition.
17b. The composition of embodiment 1b, 2b, 3b, 4b, 5b, 6b, 7b, 8b, 9b, 10b, 11b, 12b, 13b, 14b, 15b, or 16b, wherein the composition is a household care product, personal care product, industrial product, ingestible product (e.g., food product), or pharmaceutical product.
18b. The composition of embodiment 1b, 2b, 3b, 4b, 5b, 6b, 7b, 8b, 9b, 10b, 11b, 12b, 13b, 14b, 15b, 16b, or 17b, further comprising at least one surfactant.
19b. The composition of embodiment 1b, 2b, 3b, 4b, 5b, 6b, 7b, 8b, 9b, 10b, 11b, 12b, 13b, 14b, 15b, 16b, 17b, or 18b, further comprising at least one enzyme.
20b. The composition of embodiment 19b, wherein the enzyme is a cellulase, protease, lipase, amylase, lipase, or nuclease.
21b. The composition of embodiment 1b, 2b, 3b, 4b, 5b, 6b, 7b, 8b, 9b, 10b, 11b, 12b, 13b, 14b, 15b, 16b, 17b, 18b, 19b, or 20b, further comprising at least one of a complexing agent, soil release polymer, surfactancy-boosting polymer, bleaching agent, bleach activator, bleaching catalyst, fabric conditioner, clay, foam booster, suds suppressor, anti-corrosion agent, soil-suspending agent, anti-soil re-deposition agent, dye, bactericide, tarnish inhibitor, optical brightener, perfume, saturated or unsaturated fatty acid, dye transfer-inhibiting agent, chelating agent, hueing dye, visual signaling ingredient, anti-foam, structurant, thickener, anti-caking agent, starch, sand, or gelling agent.
22b. The composition of embodiment 1b, 2b, 3b, 4b, 5b, 6b, 7b, 8b, 9b, 10b, 11b, 12b, 13b, 14b, 15b, 16b, 17b, 18b, 19b, 20b, or 21b, wherein the composition is in the form of, or comprised in, a liquid, gel, powder, hydrocolloid, granule, tablet, bead or pastille, single-compartment sachet, multi-compartment sachet, single-compartment pouch, or multi-compartment pouch.
23b. The composition of embodiment 1b, 2b, 3b, 4b, 5b, 6b, 7b, 8b, 9b, 10b, 11b, 12b, 13b, 14b, 15b, 16b, 17b, 18b, 19b, 20b, 21b, or 22b, wherein the composition is a: (a) flocculation agent, (b) viscosity modifier, (c) a latex composition, (d) a pigment-comprising composition, (e) a film or coating, (f) a fiber or fibrid, (g) a cosmetic product (e.g., hair styling product), (h) a detergent composition, (i) an adhesive composition, (j) paper, or (k) any composition/product as disclosed herein.
24b. A method of producing an ester derivative of a glucan, the method comprising: (a) contacting a glucan in a reaction with at least one esterification agent comprising a cationic organic group (cationic acyl group), wherein at least one cationic organic group (cationic acyl group) is esterified to the glucan thereby producing an ester derivative of the glucan (e.g., according to embodiment 1b, 2b, 3b, 4b, 5b, 6b, 7b, 8b, 9b, 10b, 11b, 12b, 13b, 14b, or 15b), wherein the ester derivative of the glucan has a degree of substitution (DoS) up to about 3.0 with the cationic organic group (cationic acyl group), and (b) optionally, isolating the ester derivative of the glucan produced in step (a).
25b. A composition as recited in embodiment 1b, 7b, 8b, 9b, 10b, 11b, 12b, 13b, 14b, 15b, 16b, 17b, 18b, 19b, 20b, 21b, 22b, or 23b, or a method as recited in embodiment 24b, but wherein “soy polysaccharide” is recited in place of “glucan”.
26b. A method of producing a solid composition comprising at least one glucan ester derivative (such as from embodiment 1b, 2b, 3b, 5b, 6b, 7b, 8b, 9b, 10b, 11b, 12b, 13b, 14b, or 15b), wherein the method comprises at least (a) providing a non-caustic (e.g., pH 6-8 or 6-9) aqueous composition (e.g., solution or dispersion) comprising at least one glucan ester derivative as presently disclosed, (b) putting the aqueous composition into a desired form (e.g., fiber, fibrid, film/coating, composite, extrusion), and (c) removing the liquid/solvent of the aqueous composition of step (b) (e.g., by drying, and/or if a solution is used, by raising the pH of the solution to over about 10 to cause the dissolved glucan ester to precipitate out of solution) to produce a solid composition comprising the glucan ester derivative, and (d) optionally washing and/or drying the solid composition produced in step (c)
27b. A method of styling hair, the method comprising at least steps (a) and (b), or steps (c) and (d), as follows: (a) contacting (e.g., coating) hair with a glucan ester derivative according to (or a composition according to) embodiment 1b, 2b, 3b, 4b, 5b, 6b, 7b, 8b, 9b, 10b, 11b, 12b, 13b, 14b, 15b, 16b, 17b, 18b, 19b, 20b, 21b, 22b, or 23b, thereby providing treated hair (or coated hair), and (b) putting the treated hair (or the coated hair) into a desired form (e.g., straightening, curling, or putting into any other form that is different from the form the hair was in as it existed before step [a] or [b]); or (c) putting hair into a desired form (e.g., straightening, curling, or putting into any other form that is different from the form the hair was in as it existed before step [c]), and (d) contacting (e.g., coating) the hair of step (c) with a glucan ester derivative according to (or a composition according to) embodiment 1b, 2b, 3b, 4b, 5b, 6b, 7b, 8b, 9b, 10b, 11b, 12b, 13b, 14b, 15b, 16b, 17b, 18b, 19b, 20b, 21b, 22b, or 23b, thereby providing treated hair (or coated hair); and (e) optionally, removing solvent, if present, that was used to deliver the glucan ester derivative to the hair in step (a) or (d).

### EXAMPLES

The present disclosure is further exemplified in the following Examples. It should be understood that these Examples, while indicating certain aspects herein, are given by way of illustration only. From the above discussion and these Examples, one skilled in the art can ascertain the essential characteristics of the disclosed embodiments, and without departing from the spirit and scope thereof, can make various changes and modifications to adapt the disclosed embodiments to various uses and conditions.

### Materials/Methods

### Representative Preparation of Alpha-1,3-Glucan

Alpha-1,3-glucan with ~100% alpha-1,3 glycosidic linkages can be synthesized, for example, following the procedures disclosed in U.S. Appl. Publ. No. 2014/0179913 (see Example 12 therein, for example), which is incorporated herein by reference.

As another example, a slurry of alpha-1,3-glucan was prepared from an aqueous solution (0.5 L) containing Streptococcus salivarius gtfJ enzyme (100 unit/L) as described in U.S. Patent Appl. Publ. No. 2013/0244288 (incorporated herein by reference), sucrose (100 g/L) obtained from OmniPur Sucrose (EM8550), potassium phosphate buffer (10 mM) obtained from Sigma Aldrich, and FermaSure®, an antimicrobial agent (100 ppm), obtained from DuPont adjusted to pH 5.5. The resulting enzyme reaction was maintained at 20-25° C. for 24 hours. A slurry was formed since the alpha-1,3-glucan synthesized in the reaction was aqueous-insoluble. The alpha-1,3-glucan solids were then collected using a Buchner funnel fitted with a 325-mesh screen over 40-micrometer filter paper, forming a wet cake that contained about 60-80 wt % water.

### Representative Preparation of Alpha-1,6-Glucan with Alpha-1,2 Branching

Methods to prepare alpha-1,6-glucan containing various amounts of alpha-1,2 branching are disclosed in U.S. Appl. Publ. No. 2018/0282385, which is incorporated herein by reference. Reaction parameters such as sucrose concentration, temperature, and pH can be adjusted to provide alpha-1,6-glucan having various levels of alpha-1,2-branching and molecular weight. A representative procedure for the preparation of alpha-1,2-branched alpha-1,6-glucan is provided below (containing 19% alpha-1,2-branching and 81% alpha-1,6 linkages). The 1D ¹H-NMR spectrum was used to quantify glycosidic linkage distribution. Additional samples of alpha-1,6-glucan with alpha-1,2-branching were prepared similarly. For example, one sample contained 32% alpha-1,2-branching and 68% alpha-1,6 linkages, and another contained 10% alpha-1,2-branching and 90% alpha-1,6 linkages.

Soluble alpha-1,6-glucan with about 19% alpha-1,2 branching was prepared using stepwise combination of glucosyltransferase (dextransucrase) GTF8117 and alpha-1,2 branching enzyme GTFJ18T1, according to the following procedure. A reaction mixture (2 L) comprised of sucrose (450 g/L), GTF8117 (9.4 U/mL), and 50 mM sodium acetate was adjusted to pH 5.5 and stirred at 47° C. Aliquots (0.2-1 mL) were withdrawn at predetermined times and quenched by heating at 90° C. for 15 minutes. The resulting heat-treated aliquots were passed through a 0.45-μm filter. The flow-through was analyzed by HPLC to determine the concentration of sucrose, glucose, fructose, leucrose, oligosaccharides and polysaccharides. After 23.5 hours, the reaction mixture was heated to 90° C. for 30 minutes. An aliquot of the heat-treated reaction mixture was passed through a 0.45-μm filter and the flow-through was analyzed for soluble mono/disaccharides, oligosaccharides, and polysaccharides. A major product was linear dextran with a DPw of 93.

A second reaction mixture was prepared by adding 238.2 g of sucrose and 210 ml of alpha-1,2-branching enzyme GTFJ18T1 (5.0 U/mL) to the leftover heat-treated reaction mixture that was obtained from the GTF8117 reaction described immediately above. The mixture was stirred at 30° C. with a volume of ~2.2 L. Aliquots (0.2-1 mL) were withdrawn at predetermined times and quenched by heating at 90° C. for 15 minutes. The resulting heat-treated aliquots were passed through a 0.45-μm filter. The flow-through was analyzed by HPLC to determine the concentration of sucrose, glucose, fructose, leucrose, oligosaccharides and polysaccharides. After 95 hours, the reaction mixture was heated to 90° C. for 30 minutes. An aliquot of the heat-treated reaction mixture was passed through a 0.45-μm filter and the flow-through was analyzed for soluble mono/disaccharides, oligosaccharides, and polysaccharides. Leftover heat-treated mixture was centrifuged using 1-L centrifugation bottles. The supernatant was collected and cleaned more than 200-fold using an ultrafiltration system with 1- or 5-kDa MWCO cassettes and deionized water. The cleaned oligo/polysaccharide product solution was dried. Dry sample was then analyzed by ¹H-NMR spectroscopy to determine the anomeric linkages of the oligosaccharides and polysaccharides.

### Example 1

### Synthesis of Betaine Ester Derivatives of Insoluble Alpha-Glucan

This Example demonstrates using insoluble alpha-1,3-glucan to produce various forms of betaine alpha-1,3-glucan derivative, which is a soluble cationic glucan ester.

Water-insoluble alpha-1,3-glucan (80 g [493.6 mmol]) (~100% alpha-1,3 linkages) was suspended in 2.4 L of N, N-dimethylacetamide. The temperature of the preparation was then increased to 120° C. and stirred at this temperature for two hours. After cooling to 80° C., 144 g LiCl was added to the preparation. A clear solution was formed after cooling the preparation to 70° C. Milled betaine hydrochloride (45.6 g [296.16 mmol], CAS reg. no. 590-46-5) (e.g., Sigma Aldrich Cat. no. B3501) was added to the solution, which was then stirred for 10 minutes at 70° C. Then, 57.6 g (296.16 mmol) of tosyl chloride (dehydrating agent) was added to the solution and the preparation was allowed to react for 2 hours at 70° C. A clear yellow solution was formed, which was then cooled to room temperature. A solid product was precipitated by adding 4 L of ethanol to a ⅓-portion of the solution; this was done with each portion, after which all the precipitated product was combined (one could have similarly precipitated product by adding 12 L ethanol to the entire solution in a larger vessel). The precipitated product, betaine alpha-1,3-glucan ester, was washed three times with 5 liters of ethanol per wash, and then dried under a vacuum at 40° C.

Alpha-1,3-glucan samples (~100% alpha-1,3 linkages, all water-insoluble) having different molecular weights were individually reacted as above, but using different reagent amounts to generate betaine-modified alpha-1,3-glucan ester products of various molecular weight and degree of substitution (DoS) levels. Table 1 shows various betaine alpha-1,3-glucan ester products (Samples A-F) that were successfully synthesized, all of which were readily soluble in neutral water at room temperature.

TABLE 1
Various Water-Soluble Betaine
Alpha-1,3-Glucan Ester Products

| | Mw | |
|---|---|---|
| Sample | (kDa) | DOS |
| A (PH1340) | 36 | 0.6 |
| B (PH1370) | 86.8 | 0.7 |
| C (PH1369) | 54.9 | 0.51 |
| D (PH1372) | 57.7 | 0.51 |
| E (PH1368) | 348.6 | 0.27 |
| F (PH1331) | | 0.88 |

### Example 2

### Synthesis of Betaine Ester Derivatives of Soluble Alpha-Glucan

This Example demonstrates using a soluble glucan, alpha-1,2-branched alpha-1,6-glucan, to produce various forms of betaine alpha-1,6-glucan derivative, which is a soluble cationic glucan ester.

Various soluble alpha-1,2-branched alpha-1,6-glucans were used for betaine esterification: 40 kDa alpha-1,6-glucan with 20% alpha-1,2 branching, 17 kDa alpha-1,6-glucan with 45% alpha-1,2 branching, and 300 kDa alpha-1,6-glucan with 45% alpha-1,2 branching. In each of these alpha-glucans, the alpha-1,6-glucan backbone (from which there are alpha-1,2 branches) has 100% alpha-1,6 glycosidic linkages; the listed molecular weight is that of the alpha-1,6-glucan backbone. Each alpha-1,2-branch consisted of a single (pendant) glucose unit.

For betaine esterification, each of the above branched alpha-1,6-glucans (40 g) were individually dissolved in DMAc (200 mL) at an elevated temperature (110-130° C.). Betaine hydrochloride (40 g, CAS reg. no. 590-46-5) (e.g., Sigma Aldrich Cat. no. B3501) and dicyandiamide (>20 g, dehydrating agent) were then added to commence esterification. The homogeneity of each reaction preparation was further adjusted by adding DI-water and/or CaCl₂). Each reaction was then heated under vacuum for less than 3 hours. About 80 mL of liquid was removed and crude product was precipitated therefrom in methanol and washed a few times in methanol to afford desired product (betaine-modified alpha-glucan ester) in quantitative yield. Products from these reactions were designated as BC-2 (40 kDa alpha-1,6-glucan, 20% alpha-1,2 branching, DoS 0.02), BC-4 (40 kDa alpha-1,6-glucan, 20% alpha-1,2 branching, DoS 0.04), BC-10 (17 kDa alpha-1,6-glucan, 45% alpha-1,2 branching, DoS 0.04), and BC-11 (300 kDa alpha-1,6-glucan, 45% alpha-1,2 branching, DoS 0.04).

### Example 3

### Analysis of Betaine Alpha-1,3-Glucan Ester Derivatives

Aqueous solutions of the betaine alpha-1,3-glucan ester products of Table 1 were prepared and analyzed for viscosity using a Brookfield unit (spindle S03) at room temperature (~20° C.). High molecular weight glucan esters exhibited high viscosity levels (see Table 2, e.g., product E) at relatively low concentrations, which is a desired feature in a number of applications such as industrial applications (e.g., oil and gas production, waste water treatment), personal care, and home care. In contrast, glucan ester products with low molecular weight allowed high solids aqueous solutions (~15 wt % or higher) to be made (see Table 2), which is a desired feature in coating applications (e.g., paper coating); a higher solids content minimizes the required drying time and increases overall process throughput, for example.

TABLE 2
Viscosity Analysis of Aqueous
Solutions of Betaine Alpha-1,3-
Glucan Ester Products

| | | Viscosity | Polymer |
|---|---|---|---|
| Sampleª | rpm | (cP) | wt %^{b} |
| A | 20 | 100 | 15 |
| A | 50 | 116 | 15 |
| A | 100 | 122 | 15 |
| A | 20 | 330 | 18.1 |
| A | 50 | 350 | 18.1 |
| A | 100 | 350 | 18.1 |
| A | 5 | 2260 | 25 |
| A | 10 | 2160 | 25 |
| A | 20 | 2045 | 25 |
| B | 20 | 210 | 10 |
| B | 50 | 244 | 10 |
| B | 100 | 258 | 10 |
| C | 20 | 200 | 10 |
| C | 50 | 228 | 10 |
| C | 100 | 240 | 10 |
| D | 20 | 290 | 10 |
| D | 50 | 320 | 10 |
| D | 100 | 346 | 10 |
| E | 5 | 6000 | 5 |
| E | 10 | 4320 | 5 |
| E | 20 | 3060 | 5 |

^{a}See Table 1.
^{b}Concentration of glucan ester product dissolved in water.

The effect of pH on the solubility behavior of betaine-modified alpha-1,3-glucan in water was tested. A 20 wt % solution (~1 mL) of sample A (see Table 1) in water was added to 20 ml of water containing 4000, 400, 40, or 4 ppm of NaOH (water pH was 13, 12, 11, or 10, respectively). At pH 13 and 12, the glucan ester came out of solution as hard polymer settling at the bottom (pH 13) or as a cloudy solution (pH 12). At pH 11, more of the glucan ester remained in solution (some cloudiness), while most, if not all, of the glucan ester remained in solution at pH 10. This solubility behavior of betaine-modified alpha-1,3-glucan ester was unexpected. While quaternary ammonium alpha-1,3-glucan ether (e.g., hydroxypropyl trimethyl ammonium glucan ether) becomes more soluble at high pH, the opposite was true for betaine alpha-1,3-glucan ester. This behavior offers several advantages for using betaine alpha-1,3-glucan ester in polymer processing. For example, the glucan ester can be extruded into fibers or films from water into a high pH bath. The ester chemistry can then be removed (chemically or enzymatically) if desired to yield a fully water insoluble alpha-1,3-glucan product. Thus, betaine-modified alpha-1,3-glucan ester allows for maintaining a water-based processing environment to produce products comprising non-derivatized alpha-1,3-glucan.

### Biodegradability Assay

Biodegradability was determined following the OECD 301B Ready Biodegradability CO₂ Evolution Test Guideline (see OECD, 1992. Organization for Economic Co-operation and Development, OECD 301 Ready Biodegradability. OECD Guidelines for the Testing of Chemicals, Section 3—incorporated herein by reference). In this assay, the test substance (betaine glucan ester) is the sole carbon and energy source, and under aerobic conditions, microorganisms metabolize the test substance producing CO₂ or incorporating the carbon into biomass. The amount of CO₂ produced by the test substance (corrected for the CO₂ evolved by a blank inoculum) is expressed as a percentage of the theoretical amount of CO₂ (ThCO₂) that could have been produced if the organic carbon in the test substance was completely converted to CO₂.

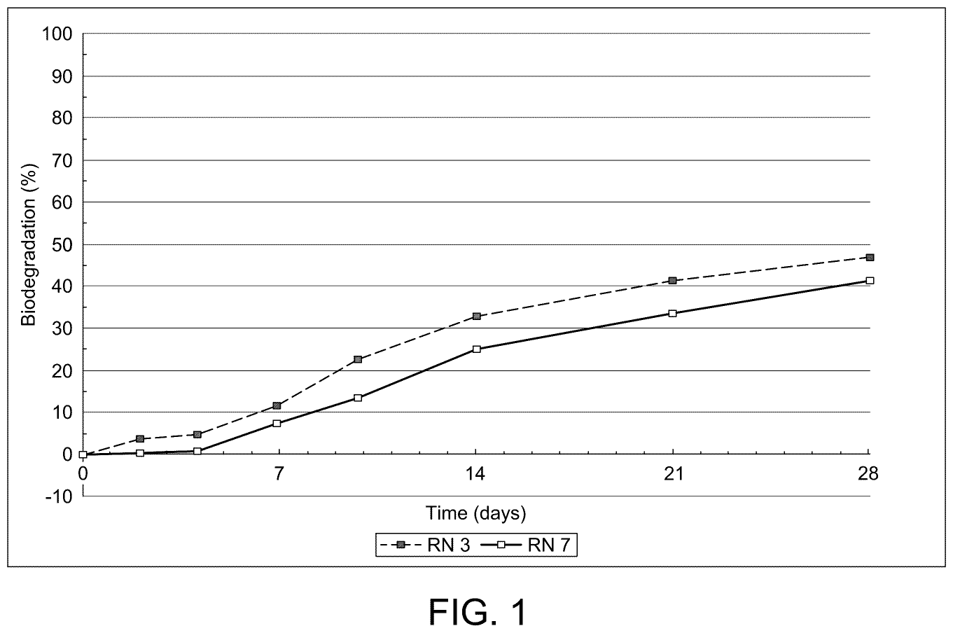

Biodegradation tests of betaine alpha-1,3-glucan ester derivative were performed following the OECD 301B test (above). The sample of glucan ester with the highest betaine group DoS (Sample F, Table 1) was analyzed for biodegradation over 28 days, and the results are shown in FIG. 1 and Table 3.

TABLE 3
Biodegradation of Betaine Alpha-1,3-
Glucan Ester Products

| | TOC^{a} | Net CO₂ | Biodegradation (%) | | |
|---|---|---|---|---|---|
| Material | (%) | (mg) | AVG | SD | REL |
| Sodium acetate | 27.9 | 68.2 | 83.8 | 1.6 | 100.0 |
| (reference) | | | | | |
| Betaine-modified | 40.4 | 52.1 | 44.0 | 3.9 | 52.5 |
| alpha 1,3 glucan | | | | | |
| (Sample F, Table 1) | | | | | |

^{a}Abbreviations: TOC (total organic carbon content), AVG (average), SD (standard deviation), REL (relative to reference)

Sample F exhibited significant biodegradability within 28 days of initiating the test (FIG. 1, Table 3). This result was surprising giving that the DoS (0.88) of Sample F is relatively high insofar as biodegradability is concerned: such a DoS level with a different linkage/derivatization type (e.g., ether-linked carboxymethyl group or ether-linked hydroxypropyl trimethyl ammonium group) typically would greatly inhibit biodegradability. The enhanced biodegradability feature of betaine alpha-1,3-glucan ester derivatives herein highly enables their use in applications where bioaccumulation of polymers is undesired (e.g., water environments).

### Example 4

### Use of Betaine-Modified Glucan Ester in Beauty Care—Hair Styling Application

In this Example, betaine alpha-1,3-glucan esters and betaine alpha-1,2-branched alpha-1,6-glucan esters were tested for features pertinent to hair styling applications. These different cationic glucan esters, which are listed in Table 4, were tested along with a negative control (no glucan derivative used) and a positive control (cationic glucan ether) for these applications.

TABLE 4

| | | | | Hair |
|---|---|---|---|---|
| | | Film Feature | | Tress |
| | | | coherent, | Height |
| | Turbidity | clear or | tacky, or | Changeª |
| Sample | (NTU) | opaque | crystalline | (cm) |
| Negative control | | | | 6.5 |
| No glucan derivative | | | | |
| Positive control | 2.1 | clear | coherent | 0 |
| 185 kDa alpha-1,6-glucan w/ | | | | |
| 5% alpha-1,2 branching | | | | |
| DoS 0.07 w/ hydroxypropyl | | | | |
| trimethylammonium (ether) | | | | |
| Betaine-modified glucan ester | | | | |
| Alpha-1,3-glucan, DoS 0.27 | Not fully | | | 6.5 |
| (Sample E, Table 1, Example 1) | dissolved | | | |
| Alpha-1,3-glucan, DoS 0.51 | 2.2 | clear | coherent | 0.5 |
| (Sample C, Table 1, Example 1) | | | | |
| Alpha-1,3-glucan, DoS 0.7 | 2.0 | clear | coherent | 0 |
| (Sample B, Table 1, Example 1) | | | | |
| Alpha-1,3-glucan, DoS 0.51 | 2.6 | clear | coherent | 0.5 |
| (Sample D, Table 1, Example 1) | | | | |
| 40 kDa alpha-1,6-glucan w/ | 20.5 | Somewhat | coherent | 2.2 |
| 20% alpha-1,2 branching | | clear (surface | | |
| DoS 0.02 | | not smooth) | | |
| (BC-2, Example 2) | | | | |
| 40 kDa alpha-1,6-glucan w/ | 1.8 | clear | coherent | 1 |
| 20% alpha-1,2 branching | | | | |
| DOS 0.04 | | | | |
| (BC-4, Example 2) | | | | |
| 17 kDa alpha-1,6-glucan w/ | 3.1 | clear | coherent | 0 |
| 45% alpha-1,2 branching | | | | |
| DoS 0.04 | | | | |
| (BC-10, Example 2) | | | | |
| 300 kDa alpha-1,6-glucan w/ | 2.4 | clear | coherent | 0 |
| 45% alpha-1,2 branching | | | | |
| DoS 0.04 | | | | |
| (BC-11, Example 2) | | | | |

^{a}Hair tress height change following styling with 1:1 ethanol/water with 1 wt % glucan derivative sample. Little or no change reflects effective hair styling (i.e., applied glucan derivative maintains bend in hair tress). Refer to text below.

Each testing sample was fully dissolved in ethanol/water (1:1) mixture at 1 wt %. The turbidity of the solution was then measured in nephelometric turbidity units (NTU) using a calibrated turbidimeter (HACH 2100AN Turbidimeter). The solution was then poured into a Petri dish and allowed to evaporate overnight at room temperature. Each resulting film was examined for quality. Good solubility and high quality film formation were observed for several of the tested betaine-modified glucan ester samples (see Table 4) as indicated, respectively, by low solution turbidity and ability to form a clear film. These features are believed to render a material as useful in hair styling products—e.g., can allow clear and transparent application on hair to provide hair styling hold while avoiding an unclean look.

In a curl retention test, ~1 gram of each solution (Table 4) was applied on a hair tress (8″ RINBOOOL Hair swatches). The resulting hair tress was dried at room temperature overnight with half of the hair tress curled back in a >90 degree angle. Each hair tress was then hung in a 45° C. oven and heated for 3 hours. The height of the curled half of each hair tress was then measured and compared to the height of the tress as it existed before the hanging (Table 4). In the control experiment, the height of the curled half of the hair tress changed by 6.5 cm. However, for several hair tresses treated with a betaine-modified glucan ester sample, the height of the curled half of the hair tresses did not change, or changed very little (see Table 4), thereby indicating significant hair styling retention.

### Example 5

### Use of Betaine-Modified Glucan Ester in Coatings

In this Example, betaine alpha-1,3-glucan ester was used to coat paper. This coating provided an oil/grease barrier to the paper.

Betaine alpha-1,3-glucan ester (Sample A, Table 1) was applied on paper substrates as a solution, prepared by dissolving betaine alpha-1,3-glucan powder at 10 wt % or 20 wt % in distilled water. For example, to prepare 50 grams of a 10 wt % formulation, 5 grams of the betaine glucan powder was dissolved in 45 grams of water. Each preparation was stirred at room temperature using a magnetic stir bar until all the powder was dissolved. Alternatively, a laboratory blender could be used if desired. The viscosity of each solution increased as the betaine glucan dissolved in the water.

Once the betaine alpha-1,3-glucan powder was fully dissolved, the resulting solution was applied onto foldable boxboard paper substrate (METSABOARD CLASSIC FBB, 235 gsm grammage, 0.425 mm thickness) using an automatic rod coater with a heating module (model PROCEQ ZAA 2600.A, Zehntner Testing Instruments). Different rods (Zehntner) were used to provide a desired coating thickness and grammage to the paper substrate: rod #3 (reference ACC378.006, wet thickness of 6.9 μm) and rod #14 (reference ACC378.032, wet thickness of 32.0 μm). The coating speed was set at 20 mm/min, and the coatings were applied at room temperature. The coated paper was dried overnight under ambient conditions. Lower times would also be possible for drying, and drying could optionally be performed in an oven at, for example, 60° C. for 10 minutes. In one test, this procedure was applied against the pre-treated side of the boxboard paper (pre-treatment by the manufacturer for printing), while this procedure was applied against the backside (non-printing side) of a separate piece of the boxboard paper in an otherwise duplicate test.

The oil barrier performance of each betaine alpha-1,3-glucan-coated paper substrate was assessed using a 60-second Cobb Unger oil test (ISO 535, TAPPI T441, SCAN P 12, EN 20535, DIN 53132, incorporated herein by reference). The results of this analysis are shown in Table 5. As compared to the negative control references (paper with no coating), paper that was coated with betaine alpha-1,3-glucan ester using either the 10 wt % and 20 wt % solutions exhibited significant oil barrier function (Table 5).

TABLE 5

| | | Coating | Cobb Oil, |
|---|---|---|---|
| Boxboard | | Grammage | 60-second |
| Substrate | Coating | (gsm) | (gsm) |
| Pre-Treated | none | 0 | 10.2 ± 2.4 |
| Side | 10 wt %, rod #3 | 4.4 ± 0.9 | 1.4 ± 0.6 |
| | 10 wt %, rod #14 | 6.0 ± 2.1 | 1.1 ± 0.2 |
| | 20 wt %, rod #3 | 1.4 ± 0.6 | 1.1 ± 1.1 |
| | 20 wt %, rod #14 | 7.5 ± 1.6 | 2.5 ± 2.1 |
| Untreated | none | 0 | 76.5 ± 8.8 |
| Side | 10 wt %, rod #3 | 3.5 ± 1.9 | 2.9 ± 0.3 |
| | 10 wt %, rod #14 | 4.7 ± 3.6 | 2.4 ± 0.4 |
| | 20 wt %, rod #3 | 3.2 ± 2.6 | 4.8 ± 0.8 |
| | 20 wt %, rod #14 | 9.7 ± 1.9 | 2.4 ± 0.7 |

gsm, gram per square meter.

### Example 6

### Two-Step Process for Synthesis of Amphiphilic Glucan Ester Derivatives Containing Cationic and Hydrophobic Substitution Groups

This Example demonstrates a two-step process using a water-soluble glucan, alpha-1,2-branched alpha-1,6-glucan, to produce a multifunctional amphiphilic alpha-glucan ester derivative. In particular, an alpha-glucan derivative substituted with betaine, benzoyl, lauroyl and acetyl groups was produced.

### Step-1. Synthesis of Hydrophobic Glucan Ester Derivative Containing Benzoyl and Lauroyl Substitution Groups:

Glucan powder (40 kDa alpha-1,6-glucan with 20% alpha-1,2 branching, 30 g) was dissolved in DMAc (150 mL) at 90° C. This reaction preparation was distilled at 100° C. under vacuum for one hour, which removed ~60 mL of liquid. Benzoyl chloride (17 g) and lauroyl chloride (8 g) were then added, after which the reaction preparation was stirred at 90° C. for 1.5 hours. The resulting product, benzoyl lauroyl alpha-1,2-branched alpha-1,6-glucan ester, was precipitated and purified using ethyl acetate, and obtained in quantitative yield.

### Step-2. Synthesis of Amphiphilic Glucan Ester Derivative Containing Betaine, Benzoyl and Lauroyl Substitution Groups:

The hydrophobic alpha-glucan ester product made in step-1 above (20 g) was dissolved in DMAc (100 mL) at an elevated temperature (110° C.). Betaine hydrochloride (20 g, CAS reg. no. 590-46-5) (e.g., Sigma Aldrich Cat. no. B3501), dicyandiamide (20 g), and water (3 mL) were then added. This reaction preparation was distilled at 130° C. under vacuum for one hour, which removed 40 mL of liquid. The reaction was then cooled to room temperature, after which crude product was first precipitated in acetonitrile, and then dissolved in water. The resulting aqueous solution was then subjected to ultrafiltration (MWCO 5 kDa) followed by freeze-drying to afford 5.6 gram of the product, benzoyl lauroyl betaine alpha-1,2-branched alpha-1,6-glucan ester. In particular, the product was determined by 1H-NMR analysis to have acyl group DoS values of 0.07, 0.52, 0.23, and 0.13, respectively, for betaine, benzoyl, lauroyl, and acetyl (acetyl derived from the DMAc solvent).

### Example 7

### Two-Step Process for Synthesis of Amphiphilic Glucan Ester Derivatives Containing Betaine and Hydrophobic Substitution Groups

### Step-1. Synthesis of Hydrophobic Glucan Ester Derivative Containing Benzoyl and Acetyl Substitution Groups:

Glucan powder (40 kDa alpha-1,6-glucan with 20% alpha-1,2 branching, 200 g) was dissolved in DMAc (1000 mL) at 88° C. This reaction preparation was distilled at 100° C. under vacuum for one hour, which removed 300 mL of liquid. Benzoyl chloride (110 g) was then added, after which the reaction preparation was stirred at 88° C. for 2 hours. The resulting product, benzoyl alpha-1,2-branched alpha-1,6-glucan ester, was precipitated and purified using isopropanol, and obtained in quantitative yield. In particular, the product was determined by 1H-NMR analysis to have acyl group DoS values of 0.36 and 0.14, respectively, for benzoyl and acetyl (acetyl derived from the DMAc solvent).

### Step-2. Synthesis of Amphiphilic Glucan Ester Derivative Containing Betaine, Benzoyl and Acetyl Substitution Groups:

The hydrophobic alpha-glucan ester product made in step-1 above (10 g) was dissolved in DMAc (300 mL) at an elevated temperature (90° C.). Next, betaine hydrochloride (6 g) was added. This reaction preparation was distilled at 100° C. under vacuum for one hour, which removed 30 mL of liquid. Tosyl chloride (12 g) was then added. This final reaction preparation was heated at 75° C. for one hour and then cooled to room temperature, after which the desired product was precipitated in isopropanol and washed three times with isopropanol afford benzoyl acetyl betaine alpha-1,2-branched alpha-1,6-glucan ester. In particular, the product was determined by 1H-NMR analysis to have acyl group DoS values of 0.27, 0.34, and 0.14, respectively, for betaine, benzoyl, and acetyl (acetyl derived from the DMAc solvent). The molecular weight of this final product was determined by SEC to be 10.5 kDa.

### Example 8

### Two-Step Process for Synthesis of Amphiphilic Glucan Ester Derivatives Containing Betaine, Benzoyl, Lauroyl, and Acetyl Substitution Groups

### Step-1. Synthesis of Hydrophobic Glucan Ester Derivative Containing Benzoyl, Lauroyl and Acetyl Substitution Groups:

Glucan powder (40 kDa alpha-1,6-glucan with 20% alpha-1,2 branching, 120 g) was swollen in DMAc (550 mL) at 90° C. for one hour. This reaction preparation was distilled at 100° C. under vacuum for one hour, which removed 185 mL of liquid. Benzoyl chloride (74 g) and lauroyl chloride (30 gram) were then added, after which the reaction preparation was stirred at 90° C. for one hour and 45 minutes. The resulting product, benzoyl lauroyl acetyl alpha-1,2-branched alpha-1,6-glucan ester, was precipitated and purified using isopropanol, and obtained in quantitative yield. In particular, the product was determined by 1H-NMR analysis to have acyl group DoS values of 0.64, 0.12, and 0.08, respectively, for benzoyl, lauroyl, and acetyl (acetyl derived from the DMAc solvent).

### Step-2. Synthesis of Amphiphilic Glucan Ester Derivative Containing Betaine, Benzoyl, Lauroyl and Acetyl Substitution Groups:

The hydrophobic alpha-glucan ester product made in step-1 above (10 g) was dissolved in DMAc (300 mL) at an elevated temperature (90° C.). Next, betaine hydrochloride (6 g) was added. This reaction preparation was distilled at 100° C. under vacuum for one hour, which removed 30 mL of liquid. Tosyl chloride (12 g) was then added. This final reaction preparation was heated at 75° C. for one hour and then cooled to room temperature, after which the desired product was precipitated in isopropanol and washed three times to afford benzoyl lauroyl acetyl betaine alpha-1,2-branched alpha-1,6-glucan ester in quantitative yield. In particular, the product was determined by 1H-NMR analysis to have acyl group DoS values of 0.13, 0.34, 0.03 and 0.01, respectively, for betaine, benzoyl, lauroyl and acetyl (acetyl derived from the DMAc solvent). The molecular weight of this final product was determined by SEC to be 11.4 kDa

### Example 9

### Two-Step Process for Synthesis of Amphiphilic Glucan Ester Derivatives Containing Betaine, Benzoyl, Lauroyl, and Acetyl Substitution Groups

### Step-1. Synthesis of Hydrophobic Glucan Ester Derivative Containing Benzoyl, Lauroyl and Acetyl Substitution Groups:

Glucan powder (40 kDa alpha-1,6-glucan with 20% alpha-1,2 branching, 120 g) was swollen in DMAc (560 mL) at 90° C. for one hour. This reaction preparation was distilled at 100° C. under vacuum for one hour, which removed 240 mL of liquid. Benzoyl chloride (74 g) and lauroyl chloride (30 g) were then added, after which the reaction preparation was stirred at 90° C. for 1.5 hours. The resulting product, benzoyl lauroyl acetyl alpha-1,2-branched alpha-1,6-glucan ester, was precipitated and purified using isopropanol, and obtained in quantitative yield. In particular, the product was determined by 1H-NMR analysis to have acyl group DoS values of 0.57, 0.04, and 0.10, respectively, for benzoyl, lauroyl, and acetyl (acetyl derived from the DMAc solvent).

### Step-2. Synthesis of Amphiphilic Glucan Ester Derivative Containing Betaine, Benzoyl, Lauroyl and Acetyl Substitution Groups:

The hydrophobic alpha-glucan ester product made in step-1 above (10 g) was dissolved in DMAc (300 mL) at an elevated temperature (90° C.). CaCl₂·2H₂O (12 g) was then added. Next, betaine hydrochloride (6 g) was added. This reaction preparation was distilled at 100° C. under vacuum for one hour, which removed 30 mL of liquid. Tosyl chloride (12 g) was then added. This final reaction preparation was heated at 75° C. for one hour and then cooled to room temperature, after which the desired product was precipitated in isopropanol and washed three times to afford benzoyl lauroyl acetyl betaine alpha-1,2-branched alpha-1,6-glucan ester in quantitative yield. In particular, the product was determined by 1H-NMR analysis to have acyl group DoS values of 0.28, 0.39, 0.02 and 0.12, respectively, for betaine, benzoyl, lauroyl and acetyl (acetyl derived from the DMAc solvent). The molecular weight of this final product was determined by SEC to be 8.1 kDa.

### Example 10

### One-Pot Synthesis of Amphiphilic Glucan Ester Derivatives Containing Cationic and Hydrophobic Substitution Groups

This Example demonstrates a one-pot (single step) process using a water-soluble glucan, alpha-1,2-branched alpha-1,6-glucan, to produce a multifunctional amphiphilic alpha-glucan ester derivative. In particular, an alpha-glucan derivative substituted with betaine, benzoyl, lauroyl and acetyl groups was produced.

Glucan powder (40 kDa alpha-1,6-glucan with 20% alpha-1,2 branching, 40 g) was dissolved in DMAc (200 mL) at an elevated temperature (90° C.). CaCl₂·2H₂O (12 g) was then added. Next, betaine hydrochloride (40 g) and tosyl chloride (25 g) were added. This reaction preparation was distilled at 100° C. under vacuum for one hour, which removed 60 mL of liquid. Benzoyl chloride (15 g) and lauroyl chloride (10 g) were then added to the reaction. This final reaction preparation was heated for one hour and then cooled to room temperature, after which ~200 mL of ethanol was added. The soluble portion of the preparation was diluted with water and purified by ultrafiltration (MWCO 5 kDa), and then freeze-dried to afford 10.8 g of the product, benzoyl lauroyl betaine alpha-1,2-branched alpha-1,6-glucan ester. In particular, the product was determined by 1H-NMR analysis to have acyl group DoS values of 0.07, 0.27, 0.16, and 0.08, respectively, for betaine, benzoyl, lauroyl, and acetyl (acetyl derived from the DMAc solvent).

### Example 11

### One-Pot Synthesis of Amphiphilic Glucan Ester Derivatives Containing Betaine and Hydrophobic Substitution Groups

This Example demonstrates a one-pot (single step) process using a water-soluble glucan, alpha-1,2-branched alpha-1,6-glucan, to produce a multifunctional amphiphilic alpha-glucan ester derivative. In particular, an alpha-glucan derivative substituted with betaine, benzoyl, lauroyl and acetyl groups was produced.

Glucan powder (40 kDa alpha-1,6-glucan with 20% alpha-1,2 branching, 20 g, pre-dried at 45° C. overnight) was swollen in DMAc (350 mL) at an elevated temperature (90° C.) for one hour. After this step, betaine hydrochloride (6 g) was added. This reaction preparation was distilled at 100° C. under vacuum for one hour, which removed 30 ml of liquid. Benzoyl chloride (7 g) and lauroyl chloride (5 g) were then added. The resulting reaction preparation was heated for one hour. Then, tosyl chloride (25 g) was added. This final reaction preparation was stirred at 75° C. for one more hour, and then cooled to room temperature. Isopropanol was added to precipitate a crude product. The solid product was further washed a couple of times with isopropanol, and then dried under vacuum to afford 17.6 g of product, benzoyl lauroyl betaine alpha-1,2-branched alpha-1,6-glucan ester. In particular, the product was determined by ¹H-NMR analysis to have acyl group DoS values of 0.15, 0.05, 0.01, and 0.07, respectively, for betaine, benzoyl, lauroyl, and acetyl (acetyl derived from the DMAc solvent). The molecular weight of this final product was determined by SEC to be 53.9 kDa.

### Example 12

### One-Pot Synthesis of Amphiphilic Glucan Ester Derivatives Containing Betaine, Benzoyl, Lauroyl, and Acetyl Substitution Groups

This Example demonstrates a one-pot (single step) process using a water-soluble glucan, alpha-1,2-branched alpha-1,6-glucan, to produce a multifunctional amphiphilic alpha-glucan ester derivative. In particular, an alpha-glucan derivative substituted with betaine, benzoyl, lauroyl and acetyl groups was produced.

Glucan powder (40 kDa alpha-1,6-glucan with 20% alpha-1,2 branching, 20 g, pre-dried at 45° C. overnight) was swollen in DMAc (350 mL) at an elevated temperature (90° C.) for one hour. After this step, betaine hydrochloride (6 g) was added. This reaction preparation was distilled at 100° C. under vacuum for one hour, which removed ~30 mL of liquid. Benzoyl chloride (10 g) and lauroyl chloride (6.5 g) were then added. The resulting reaction preparation was heated for one hour. Then, tosyl chloride (12 g) was added. This final reaction preparation was stirred at 75° C. for one more hour, and then cooled to room temperature. Isopropanol was added to precipitate a crude product. The solid product was further washed a couple of times with isopropanol, and then dried under vacuum to afford ~18 g of product, benzoyl lauroyl betaine alpha-1,2-branched alpha-1,6-glucan ester. In particular, the product was determined by 1H-NMR analysis to have acyl group DoS values of 0.20, 0.09, 0.22, and 0.19, respectively, for betaine, benzoyl, lauroyl, and acetyl (acetyl derived from the DMAc solvent). The molecular weight of this final product was determined by SEC to be 10.4 kDa.

### Example 13

### Use of Amphiphilic Ester Derivatives in Beauty Care-Hair Styling Application

In this Example, amphiphilic alpha-1,2-branched alpha-1,6-glucan esters were tested for features pertinent to hair styling applications. In particular, two different amphiphilic glucan esters produced above in Examples 6 and 10 (both being benzoyl lauroyl betaine alpha-1,2-branched alpha-1,6-glucan) were tested for these applications, along with a negative control (no glucan derivative used).

Each glucan ester test sample was fully dissolved in an ethanol/water (3:1) mixture at 4 wt %. The turbidity of the solution was then measured in nephelometric turbidity units (NTU) using a calibrated turbidimeter (HACH 2100AN Turbidimeter). The turbidity measurements for each sample are listed in Table 6.

In a curl retention test, ~0.5 gram of each solution was applied on a hair tress (8″ RINBOOOL hair swatches). The resulting hair tress was dried at room temperature overnight with half of the hair tress curled back in a >90 degree angle. The height of the curled half of each hair tress was then measured and compared to the height of the tress as it existed before drying. In the control experiment, the height of the curled half of the hair tress changed by 6.0 cm. As a comparison, for hair tresses treated with either of the amphiphilic glucan ester derivatives produced in Examples 6 (Step 2 product) or 10 (one-pot product), the height of the curled half of each hair tress changed by much less (Table 6), thereby indicating significant hair styling retention.

TABLE 6

| | | Hair Tress |
|---|---|---|
| | | Height |
| | Turbidity | Change^{a} |
| Sample | (NTU) | (cm) |
| Negative control | | 6.0 |
| No glucan derivative | | |
| ethanol/water (19:1 mixture) | | |
| Amphiphilic Glucan Ester | | |
| Product of Example 6 (Step 2) | 96 | 2.0 |
| 4 wt % in ethanol/water (3:1 mixture v/v) | | |
| Product of Example 10 | 16 | 1.0 |
| 4 wt % in ethanol/water (3:1 mixture v/v) | | |

^{a}Hair tress height change following styling with 3:1 ethanol/water having 4 wt % glucan ester derivative sample. Little or no change reflects effective hair styling (i.e., applied glucan derivative maintains bend in hair tress). Refer to above text.

What is claimed is:

1. A composition comprising an ester derivative of a glucan, wherein the glucan has a degree of substitution (DoS) up to about 3.0 with at least two organic groups that are individually ester-linked to the glucan, wherein
(i) at least one of the organic groups is a cationic organic group, and
(ii) at least one of the organic groups is a hydrophobic organic group.

2. The composition of claim 1, wherein the glucan is an alpha-glucan.

3. The composition of claim 2, wherein at least about 50% of the glycosidic linkages of the alpha-glucan are alpha-1,3 linkages.

4. The composition of claim 2, wherein at least about 50% of the glycosidic linkages of the alpha-glucan are alpha-1,6 linkages, optionally wherein the alpha-glucan comprises at least 1% alpha-1,2 and/or alpha-1,3 branches.

5. The composition of claim 1, wherein the glucan has a weight-average degree of polymerization (DPw) of at least 6.

6. The composition of claim 1, wherein the DoS with the at least two organic groups is about 0.005 to about 1.5.

7. The composition of claim 1, wherein the DoS with the cationic organic group is about 0.005 to about 1.5.

8. The composition of claim 1, wherein the DoS with said hydrophobic organic group is about 0.005 to about 1.5.

9. The composition of claim 1, wherein the cationic organic group comprises the structure: 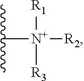
and each of R₁, R₂ and R₃ is independently a group comprising at least one carbon atom.

10. The composition of claim 9, wherein the cationic organic group comprises the structure: 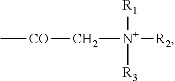
and each of R₁, R₂ and R₃ is independently a group comprising at least one carbon atom.

11. The composition of claim 1, wherein the hydrophobic organic group comprises a C₂ to C₂₆ acyl group.

12. The composition of claim 1, wherein the hydrophobic organic group comprises an aryl group.

13. The composition of claim 1, wherein the ester derivative of the glucan has a biodegradability as determined by a carbon dioxide evolution test method of at least 10% after 15 days.

14. The composition of claim 1, wherein the composition is an aqueous composition.

15. The composition of claim 1, wherein the composition is a household care product, personal care product, industrial product, ingestible product, or pharmaceutical product.

16. The composition of claim 1, further comprising at least one surfactant.

17. A method of producing an ester derivative of a glucan, said method comprising:
(a) contacting a glucan with at least two esterification agents,
wherein at least one of the esterification agents comprises a cationic organic group,
wherein at least one of the esterification agents comprises a hydrophobic organic group,
wherein at least one cationic organic group and at least one hydrophobic organic group are esterified to the glucan, thereby producing an ester derivative of the glucan,
wherein the ester derivative of the glucan has a degree of substitution (DoS) up to about 3.0 with said cationic organic group and said hydrophobic organic group, and
(b) optionally, isolating the ester derivative of the glucan produced in step (a).

18. A method of styling hair, said method comprising at least steps (a) and (b), or steps (c) and (d), as follows:
(a) contacting hair with a glucan ester derivative according to claim 1, thereby providing treated hair, and
(b) putting the treated hair (or the coated hair) into a desired form;
or
(c) putting hair into a desired form, and
(d) contacting the hair of step (c) with a glucan ester derivative according to claim 1, thereby providing treated hair;
and
(e) optionally, removing solvent, if present, that was used to deliver the glucan ester derivative to the hair in step (a) or (d).
